(19) United States
(12) Patent Application Publication
(10) Pub. No.: US 2025/0367237 A1
(43) Pub. Date: Dec. 4, 2025

(54) C-KIT BINDING PROTEINS, CHIMERIC ANTIGEN RECEPTORS, AND USES THEREOF

(71) Applicant: TeneoBio, Inc., Thousand Oaks, CA (US)

(72) Inventors: Brian Avanzino, Castro Valley, CA (US); Katherine Harris, Fremont, CA (US); Harbani Kaur Malik Chaudhry, Oakland, CA (US)

(21) Appl. No.: 19/304,317

(22) Filed: Aug. 19, 2025

Related U.S. Application Data
(63) Continuation of application No. PCT/US2024/016637, filed on Feb. 21, 2024.
(60) Provisional application No. 63/486,152, filed on Feb. 21, 2023.

(51) Int. Cl. A61K 35/17; A61K 40/11; A61K 40/31; A61K 40/42; A61P 35/00; C07K 14/725; C07K 16/28; C12N 5/0783

(52) U.S. Cl. CPC A61K 35/17; A61K 40/11; A61K 40/31; A61K 40/421; A61P 35/00; C07K 14/7051; C07K 16/2809; C12N 5/0636; A61K 2239/13; C07K 2317/31; C07K 2317/565; C07K 2317/567; C07K 2317/569; C07K 2317/92

(57) ABSTRACT

Single domain antibodies which specifically bind to c-Kit and c-Kit binding proteins, anti-c-Kit antibodies and antibody fragments thereof, antibody-drug conjugates, diagnostic agents, and chimeric antigen receptors (CARs) comprising the same are disclosed, along with CAR-expressing immune cells. Also disclosed are pharmaceutical compositions comprising any of the foregoing, uses of any of the foregoing in the treatment and/or diagnosis and/or monitoring of a disease associated with c-Kit expression, and uses of any of the foregoing in a stem cell transplantation preconditioning regimen.

### CROSS-REFERENCE TO RELATED APPLICATIONS

The present application claims the benefit of U.S. Provisional Patent Application No. 63/486,152 filed Feb. 21, 2023, each of which is incorporated herein by reference in its entirety.

### INCORPORATION-BY-REFERENCE OF SEQUENCE LISTING

The instant application contains a computer-readable Sequence Listing which has been submitted electronically in XML format and is hereby incorporated by reference in its entirety. Said XML copy, created on Feb. 16, 2024, is named “POTH-091_001WO_SeqList_ST26” and is 76,268 bytes in size.

### FIELD

Disclosed herein are single domain antibodies which specifically bind to c-Kit and c-Kit binding proteins, anti-c-Kit antibodies and antibody fragments thereof, antibody-drug conjugates, chimeric antigen receptors (CARs), and diagnostic agents comprising the same, as well as CAR-expressing immune cells. Pharmaceutical compositions comprising any of the foregoing, uses of any of the foregoing in the treatment and/or diagnosis and/or monitoring of a disease associated with c-Kit expression (e.g., c-Kit overexpression), and uses of any of the foregoing in a stem cell transplantation preconditioning regimen are also disclosed.

### BACKGROUND

c-Kit (also known as CD117 and stem cell factor receptor (SCFR); UniProt P10721; HGNC ID 6342) is a widely expressed 145 kDa type III glycoprotein receptor tyrosine kinase that binds to a cytokine known as stem cell factor (SCF), which causes certain types of blood cells to grow. c-Kit exists as a monomer on the cell surface or as a soluble protein. c-Kit is expressed on the cell surfaces of hematopoietic progenitor stem cells, mast cells, germ cells, and melanocytes (Ratajczak et al., Blood. 1995 Sep. 15; 86(6):2161-7), and c-Kit signaling though the Ras/MAPK and PI3K/ATK signaling pathways is crucial for hematopoiesis, melanogenesis, and gametogenesis (Lennartsson et al. Oncogene. 1999 Sep. 30; 18(40):5546-5; Lev and Yarden, PNAS. 1992 Jan. 15; 89(2):678-82). Binding of the dimeric ligand SCF to c-Kit induces a conformational change in c-Kit, homodimer formation, and activation that promotes survival, differentiation, and mobilization of hematopoietic progenitor cells of several lineages.

c-Kit may be found in higher than normal amounts, or in a changed form, on some cancer cell types, including gastrointestinal stromal tumor (GIST) cells and melanoma cells. Activating mutations in c-Kit are associated with several solid and liquid tumors, including small cell lung cancer (SCLC), GISTs, melanoma, and acute myeloid leukemia (AML). At least two different c-Kit isoforms are produced by alternative splicing, where the isoforms differ by the presence or absence of the four amino acid GNNK motif in the extracellular domain (Zhu et al., Leuk Lymphoma. 1994 February; 12(5-6):441-7). While both c-Kit isoforms bind to SCF, the GNNK negative (GNNK−) variant leads to higher levels of downstream signaling and is associated with tumor growth in mice (Caruana et al., Oncogene. 1999 Sep. 30; 18(40):5573-81; Phung et al., Cell Signal. 2013 November; 25(11):2231-8).

The small molecule c-Kit inhibitor imatinib is approved for the treatment of metastatic GISTs. However, some tumors become refractory to imatinib, with resistance often developing due to the occurrence of primary and secondary mutations. While imatinib treatment has an about 70% initial response rate for GISTs containing primary mutations, acquired resistance develops in about 40-50% of cases within an average of about two years.

Accordingly, there is a need for additional and effective therapeutic treatments and diagnostic agents for diseases associated with c-Kit expression, such as, e.g., GISTs.

### DETAILED DESCRIPTION

Disclosed herein is a single domain antibody which specifically binds to c-Kit, such as, e.g., human c-Kit.

In some embodiments, the single domain antibody comprises a heavy chain variable (VH) region comprising:
(i) a VH complementarity determining region one (CDR1) comprising the sequence

(SEQ ID NO: 53)
G X₁ T X₂ X₃ X₄ Y A,

wherein X₁ is F or L; X₂ is F or I; X₃ is D or S; and X₄ is S or T;
(ii) a VH CDR2 comprising the sequence

(SEQ ID NO: 71)
I S X₅ X₆ G X₇ X₈ T,

wherein X₅ is V or T, X₆ is R or G; X₇ is G or S; and X₈ is S or R; and
(iii) a VH CDR3 comprising the sequence

(SEQ ID NO: 54)
A T G Y D X₉ S G X₁₀ Y Y G G F D Y,

wherein X₉ is S or P; and X₁₀ is Y or H.

In some embodiments, the single domain antibody comprises a heavy chain variable (VH) region in which the full set of VH CDRs 1, 2, and 3 (combined) has at least 80% (such as, e.g., 80%, 85%, 90%, 95%, at least 85%, at least 90%, at least 95%) sequence identity to the VH CDRs 1, 2, and 3 of any one of SEQ ID NOs: 14-18. In some embodiments, the single domain antibody comprises a heavy chain variable (VH) region in which the full set of VH CDRs 1, 2, and 3 (combined) has at least 85% (such as, e.g., 85%, 90^{%}, 95%, at least 90%, at least 95%) sequence identity to the VH CDRs 1, 2, and 3 of any one of SEQ ID NOs: 14-18. In some embodiments, the single domain antibody comprises a heavy chain variable (VH) region in which the full set of VH CDRs 1, 2, and 3 (combined) has at least 90% (such as, e.g., 90%, 95%, at least 95%) sequence identity to the VH CDRs 1, 2, and 3 of any one of SEQ ID NOs: 14-18. In some embodiments, the single domain antibody comprises a heavy chain variable (VH) region in which the full set of VH CDRs 1, 2, and 3 (combined) has at least 95% sequence identity to the VH CDRs 1, 2, and 3 of any one of SEQ ID NOs: 14-18.

In some embodiments, the single domain antibody comprises a heavy chain variable (VH) region comprising:
(i) a VH complementarity determining region one (CDR1) comprising a sequence having at most two (e.g., one, two, zero) amino acid modifications relative to SEQ ID NO: 1, SEQ ID NO: 2, or SEQ ID NO: 3;
(ii) a VH CDR2 comprising a sequence having at most two (e.g., one, two, zero) amino acid modifications relative to SEQ ID NO: 4, SEQ ID NO: 5, SEQ ID NO: 6, or SEQ ID NO: 7; and
(iii) a VH CDR3 comprising a sequence having at most two (e.g., one, two, zero) amino acid modifications relative to SEQ ID NO: 8, SEQ ID NO: 9, or SEQ ID NO: 10.

In some embodiments, the single domain antibody comprises a heavy chain variable (VH) region comprising a VH complementarity determining region one (CDR1) comprising a sequence having at most two (e.g., one, two, zero) amino acid modifications relative to SEQ ID NO: 11; a VH CDR2 comprising a sequence having at most two (e.g., one, two, zero) amino acid modifications relative to SEQ ID NO: 12; and a VH CDR3 comprising a sequence having at most two (e.g., one, two, zero) amino acid modifications relative to SEQ ID NO: 13.

In some embodiments, each amino acid modification, if any, is a conservative amino acid substitution. In some embodiments, each amino acid modification, if any, is a conservative amino acid substitution listed in Table A1.

In some embodiments, the VH CDR1 comprises a sequence having at most one amino acid modification relative to SEQ ID NO: 1, SEQ ID NO: 2, or SEQ ID NO: 3. In some embodiments, the VH CDR2 comprises a sequence having at most one amino acid modification relative to SEQ ID NO: 4, SEQ ID NO: 5, SEQ ID NO: 6, or SEQ ID NO: 7. In some embodiments, the VH CDR3 comprises a sequence having at most one amino acid modification relative to SEQ ID NO: 8, SEQ ID NO: 9, or SEQ ID NO: 10. In some embodiments, the at most one amino acid modification is an amino acid substitution. In some embodiments, the at most one amino acid modification is a conservative amino acid substitution. In some embodiments, the at most one amino acid modification is an amino acid deletion. In some embodiments, the at most one amino acid modification is an amino acid addition.

In some embodiments, the VH CDR1 comprises a sequence having at most one amino acid modification relative to SEQ ID NO: 11. In some embodiments, the VH CDR2 comprises a sequence having at most one amino acid modification relative to SEQ ID NO: 12. In some embodiments, the VH CDR3 comprises a sequence having at most one amino acid modification relative to SEQ ID NO: 13. In some embodiments, the at most one amino acid modification is an amino acid substitution. In some embodiments, the at most one amino acid modification is a conservative amino acid substitution. In some embodiments, the at most one amino acid modification is an amino acid deletion. In some embodiments, the at most one amino acid modification is an amino acid addition.

In some embodiments, the VH CDR1 comprises a sequence chosen from SEQ ID NO: 1, SEQ ID NO: 2, and SEQ ID NO: 3. In some embodiments, the VH CDR2 comprises a sequence chosen from SEQ ID NO: 4, SEQ ID NO: 5, SEQ ID NO: 6, and SEQ ID NO: 7. In some embodiments, the VH CDR3 comprises a sequence chosen from SEQ ID NO: 8, SEQ ID NO: 9, and SEQ ID NO: 10.

In some embodiments, the single domain antibody comprises a heavy chain variable (VH) region comprising:
(i) a VH complementarity determining region one (CDR1) comprising a sequence chosen from SEQ ID NO: 1, SEQ ID NO: 2, and SEQ ID NO: 3;
(ii) a VH CDR2 comprising a sequence chosen from SEQ ID NO: 4, SEQ ID NO: 5, SEQ ID NO: 6, and SEQ ID NO: 7; and
(iii) a VH CDR3 comprising a sequence chosen from SEQ ID NO: 8, SEQ ID NO: 9, and SEQ ID NO: 10.

In some embodiments, the single domain antibody comprises a heavy chain variable (VH) region comprising:
(a) a VH CDR1, a VH CDR2, and a VH CDR3 comprising the sequences of SEQ ID NOs: 1, 4, and 8, respectively;
(b) a VH CDR1, a VH CDR2, and a VH CDR3 comprising the sequences of SEQ ID NOs: 2, 5, and 9, respectively;
(c) a VH CDR1, a VH CDR2, and a VH CDR3 comprising the sequences of SEQ ID NOs: 3, 6, and 8, respectively;
(d) a VH CDR1, a VH CDR2, and a VH CDR3 comprising the sequences of SEQ ID NOs: 2, 7, and 10, respectively; or
(e) a VH CDR1, a VH CDR2, and a VH CDR3 comprising the sequences of SEQ ID NOs: 11, 12, and 13, respectively.

In some embodiments, the single domain antibody comprises a heavy chain variable (VH) region comprising the CDR1, CDR2, and CDR3 of any one of SEQ ID NOs: 14-18.

In some embodiments, the VH CDR1, VH CDR2, and VH CDR3 sequences are present in a human VH framework.

In some embodiments, the single domain antibody comprises a heavy chain variable (VH) region having at least 80% (such as, e.g., 80%, 85%, 90%, 95%, at least 85%, at least 90%, at least 95%) sequence identity to any one of SEQ ID NOs: 14-18. In some embodiments, the single domain antibody comprises a heavy chain variable (VH) region having at least 85% (such as, e.g., 85%, 90%, 95%, at least 90%, at least 95%) sequence identity to any one of SEQ ID NOs: 14-18. In some embodiments, the single domain antibody comprises a heavy chain variable (VH) region having at least 90% (such as, e.g., 90%, 95%, at least 95%) sequence identity to any one of SEQ ID NOs: 14-18. In some embodiments, the single domain antibody comprises a heavy chain variable (VH) region having at least 95% sequence identity to any one of SEQ ID NOs: 14-18.

In some embodiments, the single domain antibody comprises a heavy chain variable (VH) region chosen from SEQ ID NOs: 14-18.

In some embodiments, the single domain antibody specifically binds to human c-Kit. In some embodiments, the single domain antibody specifically binds to a GNNK− isoform of human c-Kit. In some embodiments, the single domain antibody specifically binds to a GNNK+ isoform of human c-Kit.

In some embodiments, the single domain antibody binds to human c-Kit with a K_{D} of from about 10⁻⁹ M to about 10⁻⁶ M. In some embodiments, the single domain antibody binds to a GNNK− isoform of human c-Kit with a K_{D} of from about 10⁻⁹ M to about 10⁻⁶ M. In some embodiments, the single domain antibody binds to a GNNK+ isoform of human c-Kit with a K_{D} of from about 10⁻⁹ M to about 10⁻⁶ M.

In some embodiments, the single domain antibody is an isolated single domain antibody.

Also disclosed herein is a c-Kit binding protein comprising a single domain antibody that specifically binds to c-Kit, as described herein.

In some embodiments, the c-Kit binding protein specifically binds to human c-Kit. In some embodiments, the c-Kit binding protein specifically binds to a GNNK− isoform of human c-Kit. In some embodiments, the c-Kit binding protein specifically binds to a GNNK+ isoform of human c-Kit.

In some embodiments, the c-Kit binding protein binds to human c-Kit with a K_{D} of from about 10⁻⁹ M to about 10⁻⁶ M. In some embodiments, the c-Kit binding protein binds to a GNNK− isoform of human c-Kit with a K_{D} of from about 10⁻⁹ M to about 10⁻⁶ M. In some embodiments, the c-Kit binding protein binds to a GNNK+ isoform of human c-Kit with a K_{D} of from about 10⁻⁹ M to about 10⁻⁶ M.

In some embodiments, the c-Kit binding protein further binds to one or more target antigens other than c-Kit. In some embodiments, the c-Kit binding protein is multispecific. In some embodiments, the c-Kit binding protein is bispecific.

In some embodiments, the c-Kit binding protein further specifically binds to CD3. In some embodiments, the c-Kit binding protein further specifically binds to human CD3. In some embodiments, the c-Kit binding protein further specifically binds to human CD3 epsilon. In some embodiments, the c-Kit binding protein binds to an epitope on CD3 comprising at least one residue selected from CD3 epsilon (SEQ ID NO: 69): K73 and S83; and CD3 delta (SEQ ID NO: 70) K82 and C93. In some embodiments, the epitope on CD3 comprises the region of CD3 delta defined by K82, E83, S84, T85, V86, Q87, V88, H89, Y90, R91, M92, C93. In some embodiments, the epitope on CD3 comprises the region of CD3 epsilon defined by K73, N74, 175, G76, S77, D78, E79, D80, H81, L82, S83. In some embodiments, the epitope comprises a conformational epitope with residues of both CD3 delta and CD3 epsilon. In some embodiments, the conformational epitope comprises each of residues CD3ε K73 and S83; CD3δ K82 and C93.

In some embodiments, the c-Kit binding protein further comprises a CD3-binding VH region. In some embodiments, the c-Kit binding protein further comprises a CD3-binding VH region that is paired with a light chain (VL) region.

In some embodiments, the CD3-binding VH region comprises:
(i) a VH complementarity determining region one (CDR1) comprising a sequence having at most two (such as, e.g., zero, one, or two) amino acid modifications relative to any one of SEQ ID NOs: 20-25;
(ii) a VH CDR2 comprising a sequence having at most two (such as, e.g., zero, one, or two) amino acid modifications relative to SEQ ID NO: 26; and
(iii) a VH CDR3 comprising a sequence having at most two (such as, e.g., zero, one, or two) amino acid modifications relative to any one of SEQ ID NOs: 27-30.

In some embodiments, each amino acid modification, if any, is a conservative amino acid substitution. In some embodiments, each amino acid modification, if any, is a conservative amino acid substitution listed in Table A1.

In some embodiments, the CD3-binding VH CDR1 comprises a sequence having at most one amino acid modification relative to any one of SEQ ID NO: 20-25. In some embodiments, the CD3-binding VH CDR2 comprises a sequence having at most one amino acid modification relative to SEQ ID NO: 26. In some embodiments, the CD3-binding VH CDR3 comprises a sequence having at most one amino acid modification relative to any one of SEQ ID NOs: 27-30. In some embodiments, the at most one amino acid modification is an amino acid substitution. In some embodiments, the at most one amino acid modification is a conservative amino acid substitution. In some embodiments, the at most one amino acid modification is an amino acid deletion. In some embodiments, the at most one amino acid modification is an amino acid addition.

In some embodiments, the CD3-binding VH CDR1 comprises a sequence chosen from SEQ ID NOs: 20-25. In some embodiments, the CD3-binding VH CDR2 comprises the sequence of SEQ ID NO: 26. In some embodiments, the CD3-binding VH CDR3 comprises a sequence chosen from SEQ ID NOs: 27-30.

In some embodiments, the full set of VH CDRs 1, 2, and 3 (combined) in the CD3-binding VH region has at least 80% (such as, e.g., 80%, 85%, 90%, 95%, at least 85%, at least 90%, at least 95%) sequence identity to the VH CDRs 1, 2, and 3 of any one of SEQ ID NOs: 31-48. In some embodiments, the full set of VH CDRs 1, 2, and 3 (combined) in the CD3-binding VH region has at least 85% (such as, e.g., 85%, 90%, 95%, at least 90%, at least 95%) sequence identity to the VH CDRs 1, 2, and 3 of any one of SEQ ID NOs: 31-48. In some embodiments, the full set of VH CDRs 1, 2, and 3 (combined) in the CD3-binding VH region has at least 90% (such as, e.g., 90%, 95%, at least 95%) sequence identity to the VH CDRs 1, 2, and 3 of any one of SEQ ID NOs: 31-48. In some embodiments, the full set of VH CDRs 1, 2, and 3 (combined) in the CD3-binding VH region has at least 95% sequence identity to the VH CDRs 1, 2, and 3 of any one of SEQ ID NOs: 31-48.

In some embodiments, the CD3-binding VH region comprises the CDR1, CDR2, and CDR3 of any one of SEQ ID NOs: 31-48.

In some embodiments, the CD3-binding VH region comprises:
(i) a VH complementarity determining region one (CDR1) comprising the sequence

(SEQ ID NO: 55)
G F TF X₁₁ X₁₂ Y A,

wherein X₁₁ is D, A, or H; and X₁₂ is D or N;
(ii) a VH CDR2 comprising the sequence ISWNSGSI (SEQ ID NO: 26); and
(iii) a VH CDR3 comprising the sequence A K D S R G Y G X₁₃ Y X₁₄ X₁₅ G G A Y (SEQIDNO: 56),
wherein X₁₃ is D or S; X₁₄ is R or S; and X₁₅ is L or R.

In some embodiments, the VH CDR1, VH CDR2, and VH CDR3 sequences in the CD3-binding VH region are present in a human VH framework.

In some embodiments, the CD3-binding VH region has at least 80% (such as, e.g., 80%, 85%, 90%, 95%, at least 85%, at least 90%, at least 95%) sequence identity to any one of SEQ ID NOs: 31-48. In some embodiments, the CD3-binding VH region has at least 85% (such as, e.g., 85%, 90%, 95%, at least 90%, at least 95%) sequence identity to any one of SEQ ID NOs: 31-48. In some embodiments, the CD3-binding VH region has at least 90% (such as, e.g., 90%, 95%, at least 95%) sequence identity to any one of SEQ ID NOs: 31-48. In some embodiments, the CD3-binding VH region has at least 95% sequence identity to any one of SEQ ID NOs: 31-48.

In some embodiments, the CD3-binding VH region comprises:
(a) a VH CDR1, a VH CDR2, and a VH CDR3 comprising the sequences of SEQ ID NOs: 20, 26, and 27, respectively;
(b) a VH CDR1, a VH CDR2, and a VH CDR3 comprising the sequences of SEQ ID NOs: 20, 26, and 28, respectively;
(c) a VH CDR1, a VH CDR2, and a VH CDR3 comprising the sequences of SEQ ID NOs: 20, 26, and 29, respectively;
(d) a VH CDR1, a VH CDR2, and a VH CDR3 comprising the sequences of SEQ ID NOs: 21, 26, and 28, respectively;
(e) a VH CDR1, a VH CDR2, and a VH CDR3 comprising the sequences of SEQ ID NOs: 22, 26, and 28, respectively;
(f) a VH CDR1, a VH CDR2, and a VH CDR3 comprising the sequences of SEQ ID NOs: 23, 26, and 28, respectively;
(g) a VH CDR1, a VH CDR2, and a VH CDR3 comprising the sequences of SEQ ID NOs: 24, 26, and 28, respectively;
(h) a VH CDR1, a VH CDR2, and a VH CDR3 comprising the sequences of SEQ ID NOs: 20, 26, and 30, respectively;
(i) a VH CDR1, a VH CDR2, and a VH CDR3 comprising the sequences of SEQ ID NOs: 25, 26, and 29, respectively; or
(j) a VH CDR1, a VH CDR2, and a VH CDR3 comprising the sequences of SEQ ID NOs: 24, 26, and 29, respectively.

In some embodiments, the light chain variable region comprises the VL CDR1, VL CDR2, and VL CDR3 of SEQ ID NO: 52. In some embodiments, the light chain variable region comprises a VL CDR1, a VL CDR2, and a VL CDR3 comprising the sequence of SEQ ID NOs: 49, 50, and 51, respectively. In some embodiments, the VL CDR1, VL CDR2, and VL CDR3 sequences are present in a human VH framework.

In some embodiments, the light chain variable region has at least 80% (such as, e.g., 80%, 85%, 90%, 95%, at least 85%, at least 90%, at least 95%) sequence identity to SEQ ID NO: 52. In some embodiments, the light chain variable region has at least 85% (such as, e.g., 85%, 90%, 95%, at least 90%, at least 95%) sequence identity to SEQ ID NO: 52. In some embodiments, the light chain variable region has at least 90% (such as, e.g., 90%, 95%, at least 95%) sequence identity to SEQ ID NO: 52. In some embodiments, the light chain variable region has at least 95% sequence identity to SEQ ID NO: 52.

In some embodiments, the c-Kit binding protein is an anti-c-Kit antibody or fragment thereof. In some embodiments, the anti-c-Kit antibody is a monoclonal antibody or fragment thereof. In some embodiments, the anti-c-Kit antibody is an isolated monoclonal antibody or fragment thereof. In some embodiments, the anti-c-Kit antibody is an scFv.

In some embodiments, the anti-c-Kit antibody is an IgG1 antibody. In some embodiments, the anti-c-Kit antibody is an IgG2 antibody. In some embodiments, the anti-c-Kit antibody is an IgG4 antibody.

In some embodiments, the c-Kit binding protein is an antibody fragment. In some embodiments, the c-Kit binding protein is a heavy chain-only antibody. In some embodiments, the c-Kit binding protein is a three-chain antibody like molecule (TCA).

In some embodiments, the anti-c-Kit antibody or fragment thereof further comprises a Fc region. In some embodiments, the anti-c-Kit antibody or fragment thereof further comprises a variant Fc region. In some embodiments, the variant Fc region comprises heterodimerizing alterations. In some embodiments, the Fc region is a silenced Fc region.

Also disclosed herein is a polynucleotide encoding a single domain antibody that specifically binds to c-Kit as described herein.

Also disclosed herein is a composition comprising one or more polynucleotide(s) encoding a c-Kit binding protein as described herein. In some embodiments, the c-Kit binding protein is an anti-c-Kit antibody or fragment thereof.

Also disclosed herein is a recombinant expression vector comprising a single domain antibody that specifically binds to c-Kit as described herein, as well as a host cell comprising the recombinant expression vector.

Also disclosed herein is one or more recombinant expression vector(s) comprising one or more polynucleotide(s) encoding a c-Kit binding protein as described herein, as well as a host cell comprising the one or more recombinant expression vector(s).

Also disclosed herein is a chimeric antigen receptor comprising a single domain antibody that specifically binds to c-Kit as described herein, as well as an immune cell (e.g., a T cell, an NK cell) expressing the chimeric antigen receptor.

Also disclosed herein is an antibody-drug conjugate comprising a single domain antibody that specifically binds to c-Kit, as described herein. In some embodiments, the antibody-drug conjugate is for use in a diagnostic application, such as, e.g., the detection or monitoring of a disease associated with c-Kit expression, such as, e.g., a cancer.

Also disclosed herein is a pharmaceutical composition comprising a c-Kit binding protein, antibody-drug conjugate, anti-c-Kit antibody, antibody fragment, or CAR-expressing immune cell and a pharmaceutically acceptable excipient.

Also disclosed herein is a method of treating a disease associated with c-Kit expression in a subject in need thereof comprising administering to the subject a therapeutically effective dose of at least one c-Kit binding protein, antibody-drug conjugate, anti-c-Kit antibody, antibody fragment, or CAR-expressing immune cell as described herein. In some embodiments, the disease associated with c-Kit expression is a cancer. In some embodiments, the cancer is chosen from small cell lung cancer (SCLC), gastrointestinal stromal tumors (GISTs), melanoma, and acute myeloid leukemia (AML).

Also disclosed herein is a method of preconditioning a subject prior to a stem cell transplant, the method comprising administering to the subject a therapeutically effective dose of at least one c-Kit binding protein, antibody-drug conjugate, anti-c-Kit antibody, antibody fragment, or CAR-expressing immune cell as described herein.

In some embodiments, the method comprises administering at least one antibody-drug conjugate as described herein. In some embodiments, the method comprises administering at least one anti-c-Kit antibody or antibody fragment as described herein. In some embodiments, the method comprises administering at least one CAR-expressing immune cell as described herein.

In some embodiments, the subject is suffering from a condition in which a stem cell transplant is considered to be beneficial, such as, e.g., a hematologic disease or a hematological malignancy, such as, e.g., myelodysplastic syndrome or leukemia. In some embodiments, the subject is suffering from acute myeloid leukemia.

In some embodiments, the stem cell transplant is an autologous stem cell transplant (ASCT). In some embodiments, the method reduces the number of stem cells, progenitor cells, and/or cancer stem cells in the subject.

In some embodiments, the stem cell transplant is a hematopoietic stem cell transplant (HCST). In some embodiments, the HCST is an autologous, allogeneic, syngeneic, or xenogeneic HSCT. In some embodiments, the method reduces the number of hematopoietic stem cells, hematopoietic progenitor cells, and/or hematopoietic cancer stem cells in the subject.

In some embodiments, the method replaces a preconditioning regimen comprising radiation therapy and/or busulfan therapy.

Also disclosed herein is a c-Kit binding protein, antibody-drug conjugate, anti-c-Kit antibody, antibody fragment, or CAR-expressing immune cell as described herein for use in the treatment of a disease associated with c-Kit expression. In some embodiments, the disease associated with c-Kit expression is a cancer. In some embodiments, the cancer is chosen from small cell lung cancer (SCLC), gastrointestinal stromal tumors (GISTs), melanoma, and acute myeloid leukemia (AML).

Also disclosed herein is a use of a c-Kit binding protein, antibody-drug conjugate, anti-c-Kit antibody, antibody fragment, or CAR-expressing immune cell as described herein in the manufacture of a medicament for the treatment of a disease associated with c-Kit expression. In some embodiments, the disease associated with c-Kit expression is a cancer. In some embodiments, the cancer is chosen from small cell lung cancer (SCLC), gastrointestinal stromal tumors (GISTs), melanoma, and acute myeloid leukemia (AML).

### Non-Limiting Example Embodiments

Without limitation, some example embodiments of this disclosure include:
E1. A single domain antibody which specifically binds to c-Kit, wherein the single domain antibody comprises a heavy chain variable (VH) region in which the full set of VH CDRs 1, 2, and 3 (combined) has at least 80% sequence identity to the VH CDRs 1, 2, and 3 of any one of SEQ ID NOs: 14-18.
E2. The single domain antibody according to E1, wherein the full set of VH CDRs 1, 2, and 3 (combined) has at least 85% sequence identity to the VH CDRs 1, 2, and 3 of any one of SEQ ID NOs: 14-18.
E3 The single domain antibody according to E1 or E2, wherein the full set of VH CDRs 1, 2, and 3 (combined) has at least 90% sequence identity to the VH CDRs 1, 2, and 3 of any one of SEQ ID NOs: 14-18.
E4. The single domain antibody according to any one of E1 to E3, wherein the full set of VH CDRs 1, 2, and 3 (combined) has at least 95% sequence identity to the VH CDRs 1, 2, and 3 of any one of SEQ ID NOs: 14-18.
E5. A single domain antibody which specifically binds to c-Kit, wherein the single domain antibody comprises a heavy chain variable (VH) region comprising:
   (i) a VH complementarity determining region one (CDR1) comprising a sequence having at most two amino acid modifications relative to SEQ ID NO: 1, SEQ ID NO: 2, or SEQ ID NO: 3;
   (ii) a VH CDR2 comprising a sequence having at most two amino acid modifications relative to SEQ ID NO: 4, SEQ ID NO: 5, SEQ ID NO: 6, or SEQ ID NO: 7; and
   (iii) a VH CDR3 comprising a sequence having at most two amino acid modifications relative to SEQ ID NO: 8, SEQ ID NO: 9, or SEQ ID NO: 10.
E6. A single domain antibody which specifically binds to c-Kit, wherein the single domain antibody comprises a heavy chain variable (VH) region comprising a VH complementarity determining region one (CDR1) comprising a sequence having at most two amino acid modifications relative to SEQ ID NO: 11; a VH CDR2 comprising a sequence having at most two amino acid modifications relative to SEQ ID NO: 12; and a VH CDR3 comprising a sequence having at most two amino acid modifications relative to SEQ ID NO: 13.
E7. The single domain antibody according to E5 or E6, wherein each amino acid modification, if any, is a conservative amino acid substitution.
E8. The single domain antibody according to E5 or E7, wherein the VH CDR1 comprises a sequence having at most one amino acid modification relative to SEQ ID NO: 1, SEQ ID NO: 2, or SEQ ID NO: 3.
E9. The single domain antibody according to any one of E5, E7, or E8, wherein the VH CDR2 comprises a sequence having at most one amino acid modification relative to SEQ ID NO: 4, SEQ ID NO: 5, SEQ ID NO: 6, or SEQ ID NO: 7.
E10. The single domain antibody according to any one of E5 or E7-E9, wherein the VH CDR3 comprises a sequence having at most one amino acid modification relative to SEQ ID NO: 8, SEQ ID NO: 9, or SEQ ID NO: 10.
E11. The single domain antibody according to E6 or E7, wherein:
   the VH CDR1 comprises a sequence having at most one amino acid modification relative to SEQ ID NO: 11; and/or
   the VH CDR2 comprises a sequence having at most one amino acid modification relative to SEQ ID NO: 12; and/or
   the VH CDR3 comprises a sequence having at most one amino acid modification relative to SEQ ID NO: 13.
E12. The single domain antibody according to any one of E8 to E11, wherein the at most one amino acid modification is an amino acid substitution.
E13. The single domain antibody according to any one of E8 to E12, wherein the at most one amino acid modification is a conservative amino acid substitution.
E14. The single domain antibody according to any one of E8 to E11, wherein the at most one amino acid modification is an amino acid deletion.
E15. The single domain antibody according to any one of E8 to E11, wherein the at most one amino acid modification is an amino acid addition.
E16. The single domain antibody according to any one of E1-E5, E7-E10, or E12-E15, wherein the VH CDR1 comprises a sequence chosen from SEQ ID NO: 1, SEQ ID NO: 2, and SEQ ID NO: 3.
E17. The single domain antibody according to any one of E1-E5, E7-E10, or E12-E16, wherein the VH CDR2 comprises a sequence chosen from SEQ ID NO: 4, SEQ ID NO: 5, SEQ ID NO: 6, and SEQ ID NO: 7.
E18. The single domain antibody according to any one of E1-E5, E7-E10, or E12-E17, wherein the VH CDR3 comprises a sequence chosen from SEQ ID NO: 8, SEQ ID NO: 9, and SEQ ID NO: 10.
E19. A single domain antibody which specifically binds to c-Kit, wherein the single domain antibody comprises a heavy chain variable (VH) region comprising:
   (i) a VH complementarity determining region one (CDR1) comprising the sequence

(SEQ ID NO: 53)
G X₁ T X₂ X₃ X₄ Y A,

wherein X₁ is F or L; X₂ is F or I; X₃ is D or S; and X₄ is S or T;
(ii) a VH CDR2 comprising the sequence

(SEQ ID NO: 71)
I S X₅ X₆ G X₇ X₈ T,

wherein X; is V or T, X₆ is R or G; X₇ is G or S; and X₈ is S or R; and
(iii) a VH CDR3 comprising the sequence

(SEQ ID NO: 54)
A T G Y D X₉ S G X₁₀ Y Y G G F D Y,

wherein X₉ is S or P; and X₁₀ is Y or H.
E20. A single domain antibody which specifically binds to c-Kit, wherein the single domain antibody comprises a heavy chain variable (VH) region comprising:
   (i) a VH complementarity determining region one (CDR1) comprising a sequence chosen from SEQ ID NO: 1, SEQ ID NO: 2, and SEQ ID NO: 3;
   (ii) a VH CDR2 comprising a sequence chosen from SEQ ID NO: 4, SEQ ID NO: 5, SEQ ID NO: 6, and SEQ ID NO: 7; and
   (iii) a VH CDR3 comprising a sequence chosen from SEQ ID NO: 8, SEQ ID NO: 9, and SEQ ID NO: 10.
E21. A single domain antibody which specifically binds to c-Kit, wherein the single domain antibody comprises a heavy chain variable (VH) region comprising:
   (a) a VH CDR1, a VH CDR2, and a VH CDR3 comprising the sequences of SEQ ID NOs: 1, 4, and 8, respectively;
   (b) a VH CDR1, a VH CDR2, and a VH CDR3 comprising the sequences of SEQ ID NOs: 2, 5, and 9, respectively;
   (c) a VH CDR1, a VH CDR2, and a VH CDR3 comprising the sequences of SEQ ID NOs: 3, 6, and 8, respectively;
   (d) a VH CDR1, a VH CDR2, and a VH CDR3 comprising the sequences of SEQ ID NOs: 2, 7, and 10, respectively; or
   (e) a VH CDR1, a VH CDR2, and a VH CDR3 comprising the sequences of SEQ ID NOs: 11, 12, and 13, respectively.
E22. A single domain antibody which specifically binds to c-Kit, wherein the single domain antibody comprises a heavy chain variable (VH) region comprising the VH CDR1, VH CDR2, and VH CDR3 of any one of SEQ ID NOs: 14-18.
E23. The single domain antibody according to any one of E1 to E22, wherein the VH CDR1, VH CDR2, and VH CDR3 sequences are present in a human VH framework.
E24. A single domain antibody which specifically binds to c-Kit, wherein the single domain antibody comprises a heavy chain variable (VH) region having at least 80% sequence identity to any one of SEQ ID NOs: 14-18.
E25. The single domain antibody according to E24, wherein the VH region has at least 85% sequence identity to any one of SEQ ID NOs: 14-18.
E26. The single domain antibody according to E24 or E25, wherein the VH region has at least 90% sequence identity to any one of SEQ ID NOs: 14-18.
E27. The single domain antibody according to any one of E24 to E26, wherein the VH region has at least 95% sequence identity to any one of SEQ ID NOs: 14-18.
E28. A single domain antibody which specifically binds to c-Kit, wherein the single domain antibody comprises a heavy chain variable (VH) region chosen from SEQ ID NOs: 14-18.
E29. The single domain antibody according to any one of E1 to E28, wherein the single domain antibody specifically binds to human c-Kit.
E30. The single domain antibody according to any one of E1 to E29, wherein the single domain antibody specifically binds to a GNNK− isoform of human c-Kit.
E31. The single domain antibody according to any one of E1 to E30, wherein the single domain antibody specifically binds to a GNNK+ isoform of human c-Kit.
E32. The single domain antibody according to any one of E1 to E31, wherein the single domain antibody binds to human c-Kit with a K_{D} of from about 10⁻⁹ M to about 10⁻⁶ M.
E33. The single domain antibody according to any one of E1 to E32, wherein the single domain antibody is an isolated single domain antibody.
E34. A c-Kit binding protein comprising the single domain antibody according to any one of E1 to E32.
E35. The c-Kit binding protein according to E34, wherein the c-Kit binding protein specifically binds to human c-Kit.
E36. The c-Kit binding protein according to E34 or E35, wherein the single domain antibody specifically binds to a GNNK− isoform of human c-Kit.
E37. The c-Kit binding protein according to any one of E34 to E36, wherein the single domain antibody specifically binds to a GNNK+ isoform of human c-Kit.
E38. The c-Kit binding protein according to any one of E34 to E37, wherein the c-Kit binding protein binds to human c-Kit with a K_{D} of from about 10⁻⁹ M to about 10⁻⁶ M.
E39. The c-Kit binding protein according to any one of E34 to E38, wherein the c-Kit binding protein further specifically binds to CD3.
E40. The c-Kit binding protein according to any one of E34 to E39, wherein the c-Kit binding protein further specifically binds to human CD3.
E41. The c-Kit binding protein according to any one of E34 to E40, wherein the c-Kit binding protein further specifically binds to human CD3 epsilon.
E42. The c-Kit binding protein according to any one of E34 to E40, wherein the c-Kit binding protein binds to an epitope on CD3 comprising at least one residue chosen from CD3 epsilon (SEQ ID NO: 69): K73 and S83; and CD3 delta (SEQ ID NO: 70) K82 and C93.
E43. The c-Kit binding protein according to E42, wherein the epitope on CD3 comprises the region of CD3 delta defined by K82, E83, S84, T85, V86, Q87, V88, H89, Y90, R91, M92, C93.
E44. The c-Kit binding protein according to E42 or E43, wherein the epitope on CD3 comprises the region of CD3 epsilon defined by K73, N74, 175, G76, S77, D78, E79, D80, H81, L82, S83.
E45. The c-Kit binding protein according to any one of E42 to E44, wherein the epitope comprises a conformational epitope with residues of both CD3 delta and CD3 epsilon.
E46. The c-Kit binding protein according to any one of E42 to E45, wherein the conformational epitope comprises each of residues CD3ε K73 and S83; CD3δ K82 and C93.
E47. The c-Kit binding protein according to any one of E34 to E46, wherein the c-Kit binding protein is a monoclonal antibody.
E48. The c-Kit binding protein according to any one of E34 to E47, wherein the c-Kit binding protein is an isolated monoclonal antibody.
E49. An antibody-drug conjugate comprising the single domain antibody according to any one of E1 to E32.
E50. An anti-c-Kit antibody comprising the single domain antibody according to any one of E1 to E32.
E51. The anti-c-Kit antibody according to E50, wherein the anti-c-Kit antibody binds to an effector cell.
E52. The anti-c-Kit antibody according to E50 or E1, wherein the anti-c-Kit antibody is multi-specific.
E53. The anti-c-Kit antibody according to any one of E50 to E52, wherein the anti-c-Kit antibody further specifically binds to a tumor-specific antigen other than c-Kit.
E54. The anti-c-Kit antibody according to any one of E50 to E53, wherein the anti-c-Kit antibody is bispecific.
E55. The anti-c-Kit antibody according to any one of E50 to E54, wherein the anti-c-Kit antibody further specifically binds to CD3.
E56. The anti-c-Kit antibody according to any one of E50 to E55, wherein the anti-c-Kit antibody further specifically binds to human CD3.
E57. The anti-c-Kit antibody according to any one of E50 to E56, wherein the anti-c-Kit antibody further specifically binds to human CD3 epsilon.
E58. The anti-c-Kit antibody according to any one of E50 to E56, wherein the anti-c-Kit antibody binds to an epitope on CD3 comprising at least one residue chosen from CD3 epsilon (SEQ ID NO: 69): K73 and S83; and CD3 delta (SEQ ID NO: 70) K82 and C93.
E59. The anti-c-Kit antibody according to E58, wherein the epitope on CD3 comprises the region of CD3 delta defined by K82, E83, S84, T85, V86, Q87, V88, H89, Y90, R91, M92, C93.
E60. The anti-c-Kit antibody according to E58 or E59, wherein the epitope on CD3 comprises the region of CD3 epsilon defined by K73, N74, 175, G76, S77, D78, E79, D80, H81, L82, S83.
E61. The anti-c-Kit antibody according to any one of E58 to E60, wherein the epitope comprises a conformational epitope with residues of both CD3 delta and CD3 epsilon.
E62. The anti-c-Kit antibody according to E61, wherein the conformational epitope comprises each of residues CD3ε K73 and S83; CD3δ K82 and C93.
E63. The anti-c-Kit antibody according to any one of E50 to E62, wherein the anti-c-Kit antibody further comprises a CD3-binding VH region.
E64. The anti-c-Kit antibody according to any one of E50 to E63, wherein the anti-c-Kit antibody is an IgG4 antibody.
E65. The anti-c-Kit antibody according to any one of E50 to E63, wherein the anti-c-Kit antibody is an IgG1 antibody.
E66. The anti-c-Kit antibody according to any one of E50 to E65, wherein the anti-c-Kit antibody further comprises a CD3-binding VH region that is paired with a light chain variable (VL) region.
E67. The anti-c-Kit antibody according to E63 or E66, wherein the CD3-binding VH region comprises:
   (i) a VH complementarity determining region one (CDR1) comprising a sequence having at most two amino acid modifications relative to any one of SEQ ID NOs: 20-25;
   (ii) a VH CDR2 comprising a sequence having at most two amino acid modifications relative to SEQ ID NO: 26; and
   (iii) a VH CDR3 comprising a sequence having at most two amino acid modifications relative to any one of SEQ ID NOs: 27-30.
E68. The anti-c-Kit antibody according to E67, wherein the CD3-binding VH CDR1 comprises a sequence having at most one amino acid modification relative to any one of SEQ ID NO: 20-25.
E69. The anti-c-Kit antibody according to E67 or E68, wherein the CD3-binding VH CDR2 comprises a sequence having at most one amino acid modification relative to SEQ ID NO: 26.
E70. The anti-c-Kit antibody according to any one of E67 to E69, wherein the CD3-binding VH CDR3 comprises a sequence having at most one amino acid modification relative to any one of SEQ ID NOs: 27-30.
E71. The anti-c-Kit antibody according to any one of E68 to E70, wherein the at most one amino acid modification is an amino acid substitution.
E72. The anti-c-Kit antibody according to any one of E68 to E71, wherein the at most one amino acid modification is a conservative amino acid substitution.
E73. The anti-c-Kit antibody according to any one of E68 to E70, wherein the at most one amino acid modification is an amino acid deletion.
E74. The anti-c-Kit antibody according to any one of E68 to E70, wherein the at most one amino acid modification is an amino acid addition.
E75. The anti-c-Kit antibody according to any one of E68 to E74, wherein the CD3-binding VH CDR1 comprises a sequence chosen from SEQ ID NOs: 20-25.
E76. The anti-c-Kit antibody according to any one of E68 to E75, wherein the CD3-binding VH CDR2 comprises the sequence of SEQ ID NO: 26.
E77. The anti-c-Kit antibody according to any one of E68 to E76, wherein the CD3-binding VH CDR3 comprises a sequence chosen from SEQ ID NOs: 27-30.
E78. The anti-c-Kit antibody according to any one of E63 or E66 to E77, wherein the full set of VH CDRs 1, 2, and 3 (combined) in the CD3-binding VH region has at least 80% sequence identity to the VH CDRs 1, 2, and 3 of any one of SEQ ID NOs: 31-48.
E79. The anti-c-Kit antibody according to any one of E63 or E66 to E78, wherein the full set of VH CDRs 1, 2, and 3 (combined) in the CD3-binding VH region has at least 85% sequence identity to the VH CDRs 1, 2, and 3 of any one of SEQ ID NOs: 31-48.
E80. The anti-c-Kit antibody according to any one of E63 or E66 to E79, wherein the full set of VH CDRs 1, 2, and 3 (combined) in the CD3-binding VH region has at least 90% sequence identity to the VH CDRs 1, 2, and 3 of any one of SEQ ID NOs: 31-48.
E81. The anti-c-Kit antibody according to any one of E63 or E66 to E80, wherein the full set of VH CDRs 1, 2, and 3 (combined) in the CD3-binding VH region has at least 95% sequence identity to the VH CDRs 1, 2, and 3 of any one of SEQ ID NOs: 31-48.
E82. The anti-c-Kit antibody according to E63 or E66, wherein the CD3-binding VH region comprises:
   (i) a VH complementarity determining region one (CDR1) comprising the sequence

(SEQ ID NO: 55)
G F T F X₁₁ X₁₂ Y A,

wherein X₁₁ is D, A, or H; and X₁₂ is D or N;
(ii) a VH CDR2 comprising the sequence ISWNSGSI (SEQ ID NO: 26); and
(iii) a VH CDR3 comprising the sequence

(SEQ ID NO: 56)
A K D S R G Y G X₁₃ Y X₁₄ X₁₅ G G A Y,

wherein X₁₃ is D or S; X₁₄ is R or S; and Xis is L or R.
E83. The anti-c-Kit antibody according to E63 or E66, wherein the CD3-binding VH region comprises the VH CDR1, VH CDR2, and VH CDR3 of any one of SEQ ID NOs: 31-48.
E84. The anti-c-Kit antibody according to any one of E63 or E66 to E83, wherein the VH CDR1, VH CDR2, and VH CDR3 sequences in the CD3-binding VH region are present in a human VH framework.
E85. The anti-c-Kit antibody according to any one of E63 or E66 to E84, wherein the CD3-binding VH region has at least 80% sequence identity to any one of SEQ ID NOs: 31-48.
E86. The anti-c-Kit antibody according to any one of E63 or E66 to E85, wherein the CD3-binding VH region has at least 85% sequence identity to any one of SEQ ID NOs: 31-48.
E87. The anti-c-Kit antibody according to any one of E63 or E66 to E86, wherein the CD3-binding VH region has at least 90% sequence identity to any one of SEQ ID NOs: 31-48.
E88. The anti-c-Kit antibody according to any one of E63 or E66 to E87, wherein the CD3-binding VH region has at least 95% sequence identity to any one of SEQ ID NOs: 31-48.
E89. The anti-c-Kit antibody according to any one of E63 or E66 to E88, wherein the CD3-binding VH region comprises:
   (a) a VH CDR1, a VH CDR2, and a VH CDR3 comprising the sequences of SEQ ID NOs: 20, 26, and 27, respectively;
   (b) a VH CDR1, a VH CDR2, and a VH CDR3 comprising the sequences of SEQ ID NOs: 20, 26, and 28, respectively;
   (c) a VH CDR1, a VH CDR2, and a VH CDR3 comprising the sequences of SEQ ID NOs: 20, 26, and 29, respectively;
   (d) a VH CDR1, a VH CDR2, and a VH CDR3 comprising the sequences of SEQ ID NOs: 21, 26, and 28, respectively;
   (e) a VH CDR1, a VH CDR2, and a VH CDR3 comprising the sequences of SEQ ID NOs: 22, 26, and 28, respectively;
   (f) a VH CDR1, a VH CDR2, and a VH CDR3 comprising the sequences of SEQ ID NOs: 23, 26, and 28, respectively;
   (g) a VH CDR1, a VH CDR2, and a VH CDR3 comprising the sequences of SEQ ID NOs: 24, 26, and 28, respectively;
   (h) a VH CDR1, a VH CDR2, and a VH CDR3 comprising the sequences of SEQ ID NOs: 20, 26, and 30, respectively;
   (i) a VH CDR1, a VH CDR2, and a VH CDR3 comprising the sequences of SEQ ID NOs: 25, 26, and 29, respectively; or
   (j) a VH CDR1, a VH CDR2, and a VH CDR3 comprising the sequences of SEQ ID NOs: 24, 26, and 29, respectively.
E90. The anti-c-Kit antibody according to E89, wherein the CD3-binding VH region comprises a VH CDR1, a VH CDR2, and a VH CDR3 comprising the sequences of SEQ ID NOs: 20, 26, and 27, respectively.
E91. The anti-c-Kit antibody according to any one of E66 to E90, wherein the light chain variable region comprises the VL CDR1, VL CDR2, and VL CDR3 of SEQ ID NO: 52.
E92. The anti-c-Kit antibody according to any one of E66 to E91, wherein the light chain variable region comprises a VL CDR1, a VL CDR2, and a VL CDR3 comprising the sequence of SEQ ID NOs: 49, 50, and 51, respectively.
E93. The anti-c-Kit antibody according to any one of E66 to E92, wherein the VL CDR1, VL CDR2, and VL CDR3 sequences are present in a human VH framework.
E94. The anti-c-Kit antibody according to any one of E66 to E93, wherein the light chain variable region has at least 80% sequence identity to SEQ ID NO: 52.
E95. The anti-c-Kit antibody according to any one of E66 to E94, wherein the light chain variable region has at least 85% sequence identity to SEQ ID NO: 52.
E96. The anti-c-Kit antibody according to any one of E66 to E95, wherein the light chain variable region has at least 90% sequence identity to SEQ ID NO: 52.
E97. The anti-c-Kit antibody according to any one of E66 to E96, wherein the light chain variable region has at least 95% sequence identity to SEQ ID NO: 52.
E98. The anti-c-Kit antibody according to any one of E50 to E97, wherein the anti-c-Kit antibody further comprises a Fc region.
E99. The anti-c-Kit antibody according to any one of E50 to E98, wherein the anti-c-Kit antibody further comprises a variant Fc region.
E100. The anti-c-Kit antibody according to E99, wherein the variant Fc region comprises heterodimerizing alterations.
E101. The anti-c-Kit antibody according to any one of E98 to E100, wherein the Fc region is a silenced Fc region.
E102. The anti-c-Kit antibody according to any one of E50 to E101, wherein the anti-c-Kit antibody specifically binds to human c-Kit.
E103. The anti-c-Kit antibody according to any one of E50 to E102, wherein the anti-c-Kit antibody specifically binds to a GNNK− isoform of human c-Kit.
E104. The anti-c-Kit antibody according to any one of E50 to E103, wherein the anti-c-Kit antibody specifically binds to a GNNK+ isoform of human c-Kit.
E105. The anti-c-Kit antibody according to any one of E50 to E104, wherein the anti-c-Kit antibody binds to human c-Kit with a K_{D} of from about 10⁻⁹ M to about 10⁻⁶ M.
E106. The anti-c-Kit antibody according to any one of E50 to E105, wherein the anti-c-Kit antibody is an isolated antibody.
E107. An antibody fragment that specifically binds to c-Kit, wherein the antibody fragment comprises a fragment of the anti-c-Kit antibody according to any one of E50 to E106.
E108. The antibody fragment according to E107, wherein the antibody fragment specifically binds to human c-Kit.
E109. The antibody fragment according to E107 or E108, wherein the antibody fragment specifically binds to a GNNK− isoform of human c-Kit.
E110. The antibody fragment according to any one of E107 to E109, wherein the antibody fragment specifically binds to a GNNK+ isoform of human c-Kit.
E111. The antibody fragment according to any one of E107 to E110, wherein the antibody fragment is an isolated antibody fragment.
E112. A chimeric antigen receptor (CAR) comprising an extracellular antigen-binding domain which specifically binds to c-Kit, wherein the extracellular antigen-binding domain comprises the single domain antibody according to any one of E1 to E32.
E113. The CAR according to E112, wherein the single domain antibody comprises a heavy chain variable (VH) region comprising a VH CDR1, a VH CDR2, and a VH CDR3 comprising the sequences of SEQ ID NOs: 2, 5, and 9, respectively.
E114. The CAR according to E112 or E113, wherein the single domain antibody comprises a heavy chain variable (VH) region of SEQ ID NO: 15.
E115. The CAR according to E112, wherein the single domain antibody comprises a heavy chain variable (VH) region comprising a VH CDR1, a VH CDR2, and a VH CDR3 comprising the sequences of SEQ ID NOs: 11, 12, and 13, respectively.
E116. The CAR according to E112 or E115, wherein the single domain antibody comprises a heavy chain variable (VH) region of SEQ ID NO: 18.
E117. The CAR according to any one of E112 to E116, further comprising a transmembrane domain and an intracellular signaling domain.
E118. The CAR according to E117, wherein the CAR further comprises a hinge between the extracellular antigen binding domain and the transmembrane domain.
E119. A chimeric antigen receptor (CAR)-expressing immune cell comprising the CAR according to any one of E112 to E118.
E120. The CAR-expressing immune cell according to E119, wherein the CAR-expressing immune cell is a T-cell or a natural killer (NK) cell.
E121. A polynucleotide encoding the single domain antibody according to any one of E1 to E33.
E122. A composition comprising one or more polynucleotide(s) encoding the c-Kit binding protein according to any one of E34 to E48.
E123. A composition comprising one or more polynucleotide(s) encoding the anti-c-Kit antibody according to any one of E50 to E106 or the antibody fragment according to any one of E107 to E111.
E124. A composition comprising one or more polynucleotide(s) encoding the CAR according to any one of E112 to E118.
E125. A recombinant expression vector comprising the polynucleotide or composition according to any one of E121 to E124.
E126. A host cell comprising the recombinant expression vector according to E125.
E127. A method of treating a disease associated with c-Kit expression in a subject in need thereof comprising administering to the subject at least one c-Kit binding protein, antibody-drug conjugate, anti-c-Kit antibody, antibody fragment, or CAR-expressing immune cell according to any one of E34 to E111, E119, or E120.
E128. A method of treating a disease associated with c-Kit expression in a subject in need thereof comprising administering to the subject a therapeutically effective dose of at least one c-Kit binding protein, antibody-drug conjugate, anti-c-Kit antibody, antibody fragment, or CAR-expressing immune cell according to any one of E34 to E111, E119, or E120.
E129. The method according to E128, wherein the disease associated with c-Kit expression is a cancer.
E130. The method according to E129, wherein the cancer is chosen from small cell lung cancer (SCLC), gastrointestinal stromal tumors (GISTs), melanoma, and acute myeloid leukemia (AML).
E131. A c-Kit binding protein, antibody-drug conjugate, anti-c-Kit antibody, antibody fragment, or CAR-expressing immune cell according to any one of E34 to E11, E119, or E120 for use in the treatment of a disease associated with c-Kit expression.
E132. The c-Kit binding protein, antibody-drug conjugate, anti-c-Kit antibody, antibody fragment, or CAR-expressing immune cell for use according to E131, wherein the disease associated with c-Kit expression is a cancer.
E133. The c-Kit binding protein, antibody-drug conjugate, anti-c-Kit antibody, antibody fragment, or CAR-expressing immune cell for use according to E132, wherein the cancer is chosen from small cell lung cancer (SCLC), gastrointestinal stromal tumors (GISTs), melanoma, and acute myeloid leukemia (AML).
E134. Use of a c-Kit binding protein, antibody-drug conjugate, anti-c-Kit antibody, antibody fragment, or CAR-expressing immune cell according to any one of E34 to E111, E119, or E120 in the manufacture of a medicament for the treatment of a disease associated with c-Kit expression.
E135. The use according to E134, wherein the disease associated with c-Kit expression is a cancer.
E136. The use according to E135, wherein the cancer is chosen from small cell lung cancer (SCLC), gastrointestinal stromal tumors (GISTs), melanoma, and acute myeloid leukemia (AML).
E137. A method of preconditioning a subject prior to a stem cell transplant, comprising administering to the subject at least one c-Kit binding protein, antibody-drug conjugate, anti-c-Kit antibody, antibody fragment, or CAR expressing immune cell according to any one of E34 to E111, E119, or E120.
E138. The method according to E137, wherein the subject is suffering from myelodysplastic syndrome or leukemia.
E139. The method according to E137 or E138, wherein the stem cell transplant is an autologous stem cell transplant (ASCT).
E140. A c-Kit binding protein, antibody-drug conjugate, anti-c-Kit antibody, antibody fragment, or CAR-expressing immune cell according to any one of E34 to E111, E119, or E120 for use in a method of preconditioning a subject prior to a stem cell transplant.
E141. The c-Kit binding protein, antibody-drug conjugate, anti-c-Kit antibody, antibody fragment, or CAR-expressing immune cell for use according to E140, wherein the subject is suffering from myelodysplastic syndrome or leukemia.
E142. The c-Kit binding protein, antibody-drug conjugate, anti-c-Kit antibody, antibody fragment, or CAR-expressing immune cell for use according to E140 or E141, wherein the stem cell transplant is an autologous stem cell transplant (ASCT).
E143. Use of a c-Kit binding protein, antibody-drug conjugate, anti-c-Kit antibody, antibody fragment, or CAR-expressing immune cell according to any one of E34 to E111, E119, or E120 in the manufacture of a medicament for use in a method of preconditioning a subject prior to a stem cell transplant.
E144. The use according to E143, wherein the subject is suffering from myelodysplastic syndrome or leukemia.
E145. The use according to E143 or E144, wherein the stem cell transplant is an autologous stem cell transplant (ASCT).
E146. A pharmaceutical composition comprising at least one c-Kit binding protein, antibody-drug conjugate, anti-c-Kit antibody, antibody fragment, or CAR-expressing immune cell according to any one of E34 to E111, E119, or E120 and a pharmaceutically acceptable excipient.

### BRIEF DESCRIPTION OF THE DRAWINGS

FIG. 1A depicts representative SEM cell binding dose curves for example single domain antibodies of the present disclosure, where the SEM cells express human GNNK+c-Kit isoform. FIG. 1B depicts representative SEM cell binding dose curves for example single domain antibodies of the present disclosure, where the SEM cells express human GNNK− c-Kit isoform. FIG. 1C depicts representative NOMO-1 cell binding dose curves for example single domain antibodies of the present disclosure, where the NOMO-1 cells express human c-Kit. FIG. 1D depicts representative CHO cell binding dose curves for example single domain antibodies of the present disclosure, where the CHO cells do not express c-Kit protein. In FIGS. 1A-1D, PE mean fluorescence intensity was plotted as a fold over background (i.e., cells incubated with secondary detection antibody only).

FIG. 2A is a schematic illustration of example CAR-T structures of various configurations comprising an anti-c-Kit extracellular binding domain(s) comprising one or more single domain antibody sequence(s) described herein.

FIG. 2B and FIG. 2C depict the in vitro cytotoxic activity of T cells transfected with an anti-c-Kit CAR.

FIGS. 3A-3C depict tumor burden as tracked over time using bioluminescent imaging (BLI) in NSG mice inoculated with a human AML cell line and treated with anti-c-Kit CAR-T cells expressing a c-Kit extracellular binding domain comprising a single VH sequence (FIGS. 3A, 3B) or two VH sequences of the present disclosure displayed in tandem or dual configuration (FIG. 3C).

### DEFINITIONS

In some embodiments, “about,” when used in connection with a measurable numerical variable, refers to the indicated value of the variable and to all values of the variable that are within the experimental error of the indicated value (e.g., within the 95% confidence interval for the mean) or +10% of the indicated value, whichever is greater. In some embodiments, numeric ranges are inclusive of the numbers defining the range (i.e., the endpoints).

Where a range of values is provided, it is understood that each intervening value, to the tenth of the unit of the lower limit unless the context clearly dictates otherwise, between the upper and lower limit of that range and any other stated or intervening value in that stated range is encompassed within the disclosure. The upper and lower limits of these smaller ranges may independently be included in the smaller ranges also encompassed within the disclosure, subject to any specifically excluded limit in the stated range. Where the stated range includes one or both of the limits, ranges excluding either or both of those included limits are also included in the disclosure.

As used herein, the terms “a” and “an” mean “one or more” unless specifically indicated otherwise. Additionally, “one or more” and “at least one” are used interchangeably herein. Furthermore, unless otherwise required by context, singular terms include pluralities and plural terms include the singular.

As used herein, the term “c-Kit” refers to a 45 kDa type III glycoprotein receptor tyrosine kinase that binds to stem cell factor (SCF) and is encoded by the KIT gene. c-Kit is also referred to as CD117 and stem cell factor receptor. The term “c-Kit” includes c-Kit proteins of any human or non-human animal species, and specifically includes human c-Kit as well as c-Kit proteins of non-human mammals. As used herein, the term “human c-Kit” includes any variants, isoforms, and species homologs of human c-Kit (UniProt P10721), regardless of its source or mode of preparation. Thus, “human c-Kit” includes human c-Kit naturally expressed by cells and c-Kit expressed on cells transfected with the human c-Kit gene. The human c-Kit sequence (UniProt P10721) is provided herein as SEQ ID NO: 19.

As used herein, the term “antibody” generally refers to a tetrameric immunoglobulin protein comprising two light chain polypeptides (such as, e.g., light chain polypeptides that are about 25 kDa each) and two heavy chain polypeptides (such as, e.g., heavy chain polypeptides that are about 50-70 kDa each). The term “light chain” or “immunoglobulin light chain,” as used herein, refers to a polypeptide comprising, from amino terminus to carboxyl terminus, a single immunoglobulin light chain variable region (VL) and a single immunoglobulin light chain constant domain (CL). The immunoglobulin light chain constant domain (CL) can be a human kappa (κ) or human lambda (λ) constant domain. The term “heavy chain” or “immunoglobulin heavy chain” refers to a polypeptide comprising, from amino terminus to carboxyl terminus, a single immunoglobulin heavy chain variable region (VH), an immunoglobulin heavy chain constant domain 1 (CH1), an immunoglobulin hinge region, an immunoglobulin heavy chain constant domain 2 (CH2), an immunoglobulin heavy chain constant domain 3 (CH3), and optionally an immunoglobulin heavy chain constant domain 4 (CH4). Heavy chains are classified as mu (μ), delta (Δ), gamma (γ), alpha (α), and epsilon (ε), and define the antibody's isotype as IgM, IgD, IgG, IgA, and IgE, respectively. The IgG-class and IgA-class antibodies are further divided into subclasses, namely, IgG1, IgG2, IgG3, and IgG4, and IgA1 and IgA2, respectively. The heavy chains in IgG, IgA, and IgD antibodies have three constant domains (CH1, CH2, and CH3), whereas the heavy chains in IgM and IgE antibodies have four constant domains (CH1, CH2, CH3, and CH4). The immunoglobulin heavy chain constant domains can be from any immunoglobulin isotype, including subtypes. The antibody chains are linked together via inter-polypeptide disulfide bonds between the CL domain and the CH1 domain (i.e., between the light and heavy chain) and between the hinge regions of the two antibody heavy chains. In some embodiments, antibodies of the present disclosure are human antibodies or humanized antibodies and can be of the IgG1-, IgG2-, IgG3-, or IgG4-type.

Variable regions of immunoglobulin chains generally exhibit the same overall structure, comprising relatively conserved framework regions (FR) joined by three hypervariable regions, more often called “complementarity determining regions” or CDRs. The CDRs from the two chains of each heavy chain and light chain pair typically are aligned by the framework regions to form a structure that binds specifically to a specific epitope on the target protein (e.g., c-Kit or CD3). From N-terminus to C-terminus, naturally-occurring light and heavy chain variable regions both typically conform with the following order of these elements: FR1, CDR1, FR2, CDR2, FR3, CDR3, and FR4. A numbering system has been devised for assigning numbers to amino acids that occupy positions in each of these domains. This numbering system is defined in Kabat Sequences of Proteins of Immunological Interest (1987 and 1991, NIH, Bethesda, MD), or Chothia & Lesk, 1987, J. Mol. Biol. 196:901-917; Chothia et al., 1989, Nature 342:878-883. The CDRs and FRs of a given antibody may be identified using this system. Other numbering systems for the amino acids in immunoglobulin chains include IMGT® (the international ImMunoGeneTics information system; Lefranc et al., Dev. Comp. Immunol. 29:185-203; 2005) and AHo (Honegger and Pluckthun, J. Mol. Biol. 309(3):657-670; 2001). In some embodiments of the present disclosure, “CDR” means a complementarity-determining region of an antibody as defined in Lefranc, M P et al., IMGT, the International ImMunoGeneTics database, Nucleic Acids Res., 27:209-212 (1999).

“Framework Region” or “FR” residues are those variable domain residues other than the hypervariable region/CDR residues as herein defined.

Antibody residues herein are numbered according to the Kabat numbering system and the EU numbering system. The Kabat numbering system is generally used when referring to a residue in the variable domain (approximately residues 1-113 of the heavy chain) (e.g., Kabat et al., Sequences of Immunological Interest. 5th Ed. Public Health Service, National Institutes of Health, Bethesda, Md. (1991)). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human IgG1 EU antibody. Unless stated otherwise herein, references to residue numbers in the variable domain of antibodies mean residue numbering by the Kabat numbering system. Unless stated otherwise herein, references to residue numbers in the constant domain of antibodies, single domain antibodies, antibody fragments, and the like mean residue numbering by the EU numbering system.

As used herein, an “anti-c-Kit antibody” is an antibody that specifically binds to c-Kit.

The term “monoclonal antibody,” as used herein, refers to an antibody obtained from a population of substantially homogeneous antibodies, i.e., the individual antibodies comprising the population are identical except for possible naturally occurring mutations that may be present in minor amounts. In contrast to polyclonal antibody preparations which typically include different antibodies directed against different determinants (epitopes), a monoclonal antibody is generally directed against a single determinant on the antigen. As non-limiting examples, monoclonal antibodies in accordance with the present disclosure can be made by the hybridoma method first described by Kohler et al. (1975) Nature 256:495, and can also be made via recombinant protein production methods (see, e.g., U.S. Pat. No. 4,816,567).

The term “human antibody,” as used herein, is intended to include antibodies having variable and constant regions derived from human germline immunoglobulin sequences. The human antibodies of the present disclosure may include amino acid residues not encoded by human germline immunoglobulin sequences (e.g., mutations introduced by random or site-specific mutagenesis in vitro or by somatic mutation in vivo). However, the term “human antibody,” as used herein, is not intended to include antibodies in which CDR sequences that are derived from the germline of another mammalian species, such as, e.g., a mouse, have been grafted onto human framework sequences.

As used herein, an “antibody fragment” generally refers to a fragment of a full-length antibody, such as, e.g., VH, VHH, VL, (s)dAb, Fv, light chain (VL-CL), Fd (VH-CH1), heavy chain, Fab, Fab′, F(ab′)₂ or “r IgG” (“half antibody” consisting of a heavy chain and a light chain) or a modified fragment of a full-length antibody, such as, e.g., triple-chain antibody-like molecule, heavy-chain only antibody, single-chain variable fragment (scFv), di-scFv or bi(s)-scFv, scFv-Fc, scFv-zipper, single-chain Fab (scFab), Fab₂, Fab₃, diabodies, single-chain diabodies, tandem diabodies (Tandabs), tandem di-scFv, tandem tri-scFv, “minibodies” exemplified by a structure which is as follows: (VH-VL-CH3)₂, (scFv-CH3)₂, ((scFv)₂-CH3+CH3), ((scFv)₂-CH3) or (scFv-CH3-scFv)₂, multibodies, such as triabodies or tetrabodies, and single domain antibodies, such as nanobodies or single variable domain antibodies comprising merely one variable region, which might be VHH, VH or VL, that specifically binds to an antigen or target independently of other variable regions or domains.

As used herein, the term “heavy chain-only antibody” refers to a dimeric immunoglobulin protein consisting of two heavy chain polypeptides (such as, e.g., heavy chain polypeptides that are about 50-70 kDa each). A “heavy chain-only antibody” is an antibody fragment that lacks the two light chain polypeptides found in a conventional antibody. In some embodiments, a “heavy chain-only antibody” is a homodimeric antibody comprising a VH antigen-binding domain and the CH2 and CH3 constant domains, in the absence of the CH1 domain. In some embodiments, a heavy chain-only antibody is composed of a variable region antigen-binding domain composed of framework 1, CDR1, framework 2, CDR2, framework 3, CDR3, and framework 4. In some embodiments, a heavy chain-only antibody is composed of an antigen-binding domain, at least part of a hinge region, and CH2 and CH3 domains. In some embodiments, a heavy chain-only antibody is composed of an antigen-binding domain, at least part of a hinge region, and a CH2 domain. In some embodiments, a heavy chain-only antibody is composed of an antigen-binding domain, at least part of a hinge region, and a CH3 domain. Heavy chain-only antibodies in which the CH2 and/or CH3 domain is truncated are also included herein. The heavy chain-only antibodies described herein may belong to the IgG subclass, but heavy chain-only antibodies belonging to other subclasses, such as IgM, IgA, IgD and IgE subclass, are also included herein. In some embodiments, a heavy chain-only antibody may belong to the IgG1, IgG2, IgG3, or IgG4 subtype, e.g., the IgG1 or IgG4 subtype. In some embodiments, a heavy chain antibody-only is of the IgG1 or IgG4 subtype, wherein one or more of the CH domains is modified to alter an effector function of the antibody. In some embodiments, a heavy chain-only antibody is of the IgG4 subtype, wherein one or more of the CH domains is modified to alter an effector function of the antibody. In some embodiments, a heavy chain-only antibody is of the IgG1 subtype, wherein one or more of the CH domains is modified to alter an effector function of the antibody. Modifications of CH domains that alter effector function are further described herein. Non-limiting examples of heavy-chain-only antibodies are described, for example, in WO2018/039180, the disclosure of which is incorporated herein by reference herein in its entirety.

As used herein, the term “single domain antibody” refers to a single polypeptide chain that contains all or part of the heavy chain variable domain or all or part of the light chain variable domain of an antibody. In some embodiments, the single domain antibody is a human single domain antibody.

As used herein, the term “three-chain antibody like molecule” or “TCA” refers to antibody-like molecules comprising, consisting essentially of, or consisting of three polypeptide subunits, two of which comprise, consist essentially of, or consist of one heavy and one light chain of a monoclonal antibody, or antigen-binding fragments of such antibody chains, comprising an antigen-binding region and at least one CH domain. This heavy chain/light chain pair has binding specificity for a first antigen. The third polypeptide subunit comprises, consists essentially of, or consists of a heavy-chain only antibody comprising an Fc portion comprising CH2 and/or CH3 and/or CH4 domains, in the absence of a CH1 domain, and one or more antigen binding domains (such as, e.g., two antigen binding domains) that binds an epitope of a second antigen or a different epitope of the first antigen, where such binding domain is derived from or has sequence identity with the variable region of an antibody heavy or light chain. Parts of such variable region may be encoded by VH and/or VL gene segments, D and J_{H} gene segments, or J_{L} gene segments. The variable region may be encoded by rearranged V_{H}DJ_{H}, V_{L}DJ_{H}, V_{H}J_{L}, or V_{L}J_{L} gene segments.

As used herein, the term “antigen-binding domain,” which is used interchangeably with “binding domain,” refers to the region of a polypeptide that contains that amino acid residues that interact with an antigen and confer on the polypeptide its specificity and affinity for the antigen. In certain embodiments, binding domains may be derived from an antibody or antigen-binding fragment thereof.

As used herein, an “antigen-binding fragment” is a portion of an antibody that lacks at least some of the amino acids present in a full-length heavy chain and/or light chain, but which is still capable of specifically binding to an antigen. An antigen-binding fragment includes, but is not limited to, a single-chain variable fragment (scFv), a nanobody (e.g. VH domain of camelid heavy chain antibodies; VHH fragment, see Cortez-Retamozo et al., Cancer Research, Vol. 64:2853-57, 2004), a Fab fragment, a Fab′ fragment, a F(ab′)₂ fragment, a Fv fragment, a Fd fragment, and a CDR fragment, and can be derived from any mammalian source, such as human, mouse, rat, rabbit, or camelid.

Papain digestion of antibodies produces two identical antigen-binding fragments, called “Fab” fragments, each with a single antigen-binding site, and a residual “Fc” fragment which contains all but the first domain of the immunoglobulin heavy chain constant region. The Fab fragment contains the variable domains from the light and heavy chains, as well as the constant domain of the light chain and the first constant domain (CH1) of the heavy chain. Thus, a “Fab fragment” is comprised of one immunoglobulin light chain (light chain variable region (VL) and constant region (CL)) and the CH1 domain and variable region (VH) of one immunoglobulin heavy chain. The heavy chain of a Fab molecule cannot form a disulfide bond with another heavy chain molecule. The “Fd fragment” comprises the VH and CH1 domains from an immunoglobulin heavy chain. The Fd fragment represents the heavy chain component of the Fab fragment.

The “Fc fragment” or “Fc region” of an immunoglobulin generally comprises two constant domains, a CH2 domain and a CH3 domain, and optionally comprises a CH4 domain. In some embodiments of the present disclosure, a c-Kit binding protein (such as, e.g., an anti-c-Kit antibody fragment, such as, e.g., a TCA or heavy chain-only antibody) comprises an Fc region from an immunoglobulin. The Fc region may be an Fc region from an IgG1, IgG2, IgG3, or IgG4 immunoglobulin. In some embodiments, the Fc region comprises CH2 and CH3 domains from a human IgG1 or human IgG2 immunoglobulin. The Fc region may retain effector function, such as C1q binding, complement dependent cytotoxicity (CDC), Fc receptor binding, antibody-dependent cell-mediated cytotoxicity (ADCC), and phagocytosis. In other embodiments, the Fc region may be modified to reduce or eliminate effector function.

A “functional Fc region” possesses an “effector function” of a native-sequence Fc region. Non-limiting examples of effector functions include C1q binding, CDC; Fc-receptor binding, ADCC, ADCP, down-regulation of cell-surface receptors (e.g., B-cell receptor), etc. Such effector functions generally require the Fc region to interact with a receptor, such as, e.g., the FcγRI; FcγRIIA; FcγRIIB1; FcγRIIB2; FcγRIIIA; FcγRIIIB receptors, and the low affinity FcRn receptor; and can be assessed using various assays known in the art.

A “dead” or “silenced” Fc is one that has been mutated to retain activity with respect to, for example, prolonging serum half-life, but which does not activate a high affinity Fc receptor, or which has a reduced affinity to an Fc receptor.

A “native-sequence Fc region” comprises an amino acid sequence identical to the amino acid sequence of an Fc region found in nature. Native-sequence human Fc regions include, for example, a native-sequence human IgG1 Fc region (non-A and A allotypes); native-sequence human IgG2 Fc region; native-sequence human IgG3 Fc region; and native-sequence human IgG4 Fc region, as well as naturally occurring variants thereof.

A “variant Fc region” comprises an amino acid sequence that differs from that of a native-sequence Fc region by virtue of at least one amino acid modification, for example, one or more (e.g., two or more, three or more, four or more) amino acid substitution(s). Illustratively, in some embodiments, the variant Fc region has at least one amino acid substitution compared to a native-sequence Fc region or to the Fc region of a parent polypeptide, e.g., from about one to about ten amino acid substitutions, e.g., from about one to about five amino acid substitutions in a native-sequence Fc region or in the Fc region of the parent polypeptide. In some embodiments, the variant Fc region herein will possess at least about 80% homology with a native-sequence Fc region and/or with an Fc region of a parent polypeptide, e.g., at least about 85% homology therewith, e.g., at least about 90% homology therewith, e.g., at least about 95% homology therewith, e.g., at least about 99% homology therewith.

As used herein, the term “heterodimerizing alterations” refers to alterations in the A and B chains of an Fc region (i.e., the two chains comprising the Fc region, wherein one chain is referred to herein as the “A” chain and the other is referred to herein as the “B” chain) that facilitate the formation of heterodimeric Fc regions, that is, Fc regions in which the A chain and the B chain of the Fc region do not have identical amino acid sequences. In some embodiments, heterodimerizing alterations can be asymmetric, that is, an A chain having a certain alteration can pair with a B chain having a different alteration. These alterations facilitate heterodimerization and disfavor homodimerization. Whether hetero- or homo-dimers have formed can be assessed, for example, by size differences as determined by polyacrylamide gel electrophoresis in situations where one polypeptide chain is a dummy Fc and the other is an scFv-Fc. One non-limiting example of such paired heterodimerizing alterations are the so-called “knobs and holes” substitutions. See, e.g., U.S. Pat. No. 7,695,936 and U.S. Patent Application Publication No. 2003/0078385. As used herein, an Fc region that comprises one pair of knobs and holes substitutions, comprises one substitution in the A chain and another in the B chain. For example, the following knobs and holes substitutions in the A and B chains of an IgG1 Fc region have been found to increase heterodimer formation as compared with that found with unmodified A and B chains and may be employed in non-limiting embodiments of this disclosure: 1) Y407T in one chain and T366Y in the other; 2) Y407A in one chain and T366W in the other; 3) F405A in one chain and T394W in the other; 4) F405W in one chain and T394S in the other; 5) Y407T in one chain and T366Y in the other; 6) T366Y and F405A in one chain and T394W and Y407T in the other; 7) T366W and F405W in one chain and T394S and Y407A in the other; 8) F405W and Y407A in one chain and T366W and T394S in the other; and 9) T366W in one polypeptide of the Fc and T366S, L368A, and Y407V in the other. Alternatively or in addition to such alterations, substitutions creating new disulfide bridges can facilitate heterodimer formation. See, e.g., U.S. Patent Application Publication No. 2003/0078385. Such alterations in an IgG1 Fc region include, but are not limited to, the following substitutions: Y349C in one Fc polypeptide chain and S354C in the other; Y349C in one Fc polypeptide chain and E356C in the other; Y349C in one Fc polypeptide chain and E357C in the other; L351C in one Fc polypeptide chain and S354C in the other; T394C in one Fe polypeptide chain and E397C in the other; or D399C in one Fc polypeptide chain and K392C in the other. Additionally or alternatively, substitutions changing the charge of a one or more residue(s), for example, in the CH3-CH3 interface, can enhance heterodimer formation, as described, for example, in WO 2009/089004, which is incorporated by reference herein. Such substitutions are referred to herein as “charge pair substitutions,” and an Fc region comprising one pair of charge pair substitutions comprises one substitution in the A chain and a different substitution in the B chain. Non-limiting examples of charge pair substitutions include the following: 1) K409D or K409E in one chain plus D399K or D399R in the other; 2) K392D or K392E in one chain plus D399K or D399R in the other; 3) K439D or K439E in one chain plus E356K or E356R in the other; and 4) K370D or K370E in one chain plus E357K or E357R in the other. In addition, the substitutions R355D, R355E, K360D, or K360R in both chains can stabilize heterodimers when used with other heterodimerizing alterations. Specific charge pair substitutions can be used either alone or with other charge pair substitutions. Specific examples of single pairs of charge pair substitutions and combinations thereof include the following: 1) K409E in one chain plus D399K in the other; 2) K409E in one chain plus D399R in the other; 3) K409D in one chain plus D399K in the other; 4) K409D in one chain plus D399R in the other; 5) K392E in one chain plus D399R in the other; 6) K392E in one chain plus D399K in the other; 7) K392D in one chain plus D399R in the other; 8) K392D in one chain plus D399K in the other; 9) K409D and K360D in one chain plus D399K and E356K in the other; 10) K409D and K370D in one chain plus D399K and E357K in the other; 11) K409D and K392D in one chain plus D399K, E356K, and E3357K in the other; 12) K409D and K392D on one chain and D399K on the other; 13) K409D and K392D on one chain plus D399K and E356K on the other; 14) K409D and K392D on one chain plus D399K and D357K on the other; 15) K409D and K370D on one chain plus D399K and D357K on the other; 16) D399K on one chain plus K409D and K360D on the other; and 17) K409D and K439D on one chain plus D399K and E356K on the other. Any of these heterodimerizing alterations can be used in polypeptides comprising variant Fc regions as described herein.

In some non-limiting embodiments, variant Fc sequences may include three amino acid substitutions in the CH2 region to reduce FcγRI binding at EU index positions 234, 235, and 237 (see Duncan et al., (1988) Nature 332:563). Two amino acid substitutions in the complement C1q binding site at EU index positions 330 and 331 reduce complement fixation (see Tao et al., J. Exp. Med. 178:661 (1993) and Canfield and Morrison, J. Exp. Med. 173:1483 (1991)). Substitution into human IgG1 or IgG2 residues at positions 233-236 and IgG4 residues at positions 327, 330 and 331 greatly reduces ADCC and CDC (see, for example, Armour K L. et al., 1999 Eur J Immunol. 29(8):2613-24; and Shields R. L. et al., 2001. J Biol Chem. 276(9):6591-604). The human IgG4 Fc amino acid sequence (UniProtKB No. P01861) is provided herein as SEQ ID NO: 76. Silenced IgG1 is described, for example, in Boesch, A. W., et al., “Highly parallel characterization of IgG Fc binding interactions.” MAbs, 2014. 6(4): p. 915-27, the disclosure of which is incorporated herein by reference in its entirety.

Other Fc variants are possible, including, without limitation, one in which a region capable of forming a disulfide bond is deleted, or in which certain amino acid residues are eliminated at the N-terminal end of a native Fc, or a methionine residue is added thereto. Thus, in some embodiments, one or more Fc portions of an antibody can comprise one or more mutations in the hinge region to eliminate disulfide bonding. In yet another embodiment, the hinge region of an Fc can be removed entirely. In still another embodiment, an antibody can comprise an Fc variant.

Further, an Fc variant can be constructed to remove or substantially reduce effector functions by substituting (mutating), deleting, or adding amino acid residues to effect complement binding or Fc receptor binding. For example, and not by way of limitation, a deletion may occur in a complement-binding site, such as a C1q-binding site. Techniques for preparing such sequence derivatives of the immunoglobulin Fc fragment are disclosed in International Patent Publication Nos. WO 97/34631 and WO 96/32478. In addition, the Fc domain may be modified by phosphorylation, sulfation, acylation, glycosylation, methylation, farnesylation, acetylation, amidation, and the like.

Antibodies and antibody fragments with reduced effector function include, but are not limited to, those with substitution of one or more of Fc region residues 238, 265, 269, 270, 297, 327 and 329 according to EU numbering (see, e.g., U.S. Pat. No. 6,737,056). In some embodiments, variant Fc regions with reduced effector function comprise substitutions at two or more of amino acid positions 265, 269, 270, 297 and 327 according to EU numbering, including the so-called “DANA” Fc mutant with substitution of residues 265 and 297 to alanine according to EU numbering (i.e., D265A and N297A according to EU numbering) (see, e.g., U.S. Pat. No. 7,332,581). In some embodiments, the variant Fc region with reduced effector function comprises the following two amino acid substitutions: D265A and N297A.

In some embodiments, effector function is reduced through a mutation in a constant region that eliminates glycosylation, e.g., an “effector-less mutation.” In some embodiments, the effector-less mutation is an N297A or a DANA mutation (D265A+N297A) in the CH2 region. Shields et al., J. Biol. Chem. 276 (9): 6591-6604 (2001). In some embodiments, the effector-less mutation is an N297G or a DANG mutation (D265A+N297G) in the CH2 region. In some embodiments, the variant Fc region lacks glycosylation at N297, e.g., the variant Fc region is a variant Fc region lacking glycosylation at N297 as described in International Patent Publication No. WO 2014/153063, which is incorporated by reference herein. Alternatively, additional mutations resulting in reduced or eliminated effector function include: K322A and L234A/L235A (LALA). Alternatively, effector function can be reduced or eliminated through production techniques, such as expression in host cells that do not glycosylate (e.g., E. coli) or in host cells which result in an altered glycosylation pattern that is ineffective or less effective at promoting effector function (e.g., Shinkawa et al., J. Biol. Chem. 278(5): 3466-3473 (2003)).

In some embodiments, the proline at position 329 (EU numbering) (P329) of a wild-type human Fc region is substituted with glycine or arginine or an amino acid residue large enough to destroy the proline sandwich within the Fc/Fcγ receptor interface, that is formed between the P329 of the Fc and tryptophan residues W87 and W110 of FcgRIII (Sondermann et al., Nature 406, 267-273 (20 Jul. 2000)). In some further embodiments, at least one further amino acid substitution in the Fc variant region is S228P, E233P, L234A, L235A, L235E, N297A, N297D, or P331S. In some embodiments, the at least one further amino acid substitution is L234A and L235A of the human IgG1 Fc region or S228P and L235E of the human IgG4 Fc region, all according to EU numbering (see, e.g., U.S. Pat. No. 8,969,526, which is incorporated by reference in its entirety).

In some embodiments, the variant Fc region has P329 of the human IgG Fc region substituted with glycine, wherein the variant Fc region comprises at least two further amino acid substitutions at L234A and L235A of the human IgG1 Fc region or S228P and L235E of the human IgG4 Fc region, and wherein the residues are numbered according to the EU numbering (see, e.g., U.S. Pat. No. 8,969,526). In some embodiments, the variant Fc region comprising the P329G, L234A and L235A (EU numbering) substitutions exhibits a reduced affinity to the human FcγRIIIA and FcγRIIA.

In some embodiments, the variant Fc region comprises a triple mutation: an amino acid substitution at position P329, a L234A, and a L235A mutation according to EU numbering (P329/LALA) (see, e.g., U.S. Pat. No. 8,969,526). In some embodiments, the variant Fc region comprises the following amino acid substitutions: P329G, L234A, and L235A according to EU numbering.

In some embodiments, an antibody or antibody fragment comprises a variant human IgG4 CH3 domain sequence comprising a T366W mutation, which can optionally be referred to herein as an IgG4 CH3 knob sequence. In some embodiments, an antibody or antibody fragment comprises a variant human IgG4 CH3 domain sequence comprising a T366S mutation, an L368A mutation, and a Y407V mutation, which can optionally be referred to herein as an IgG4 CH3 hole sequence. The IgG4 CH3 mutations described herein can be utilized in any suitable manner so as to place a “knob” on a first heavy chain constant region of a first monomer in an antibody dimer, and a “hole” on a second heavy chain constant region of a second monomer in an antibody dimer, thereby facilitating proper pairing (heterodimerization) of the desired pair of heavy chain polypeptide subunits in the antibody.

In some embodiments, an antibody or antibody fragment comprises a heavy chain polypeptide subunit comprising a variant human IgG4 Fc region comprising an S228P mutation, an F234A mutation, an L235A mutation, and a T366W mutation (knob). In some embodiments, an antibody or antibody fragment comprises a heavy chain polypeptide subunit comprising a variant human IgG4 Fc region comprising an S228P mutation, an F234A mutation, an L235A mutation, a T366S mutation, an L368A mutation, and a Y407V mutation (hole).

A “Fab′ fragment” is a Fab fragment having at the C-terminus of the CH1 domain one or more cysteine residues from the antibody hinge region.

A “F(ab′)₂ fragment” is a bivalent fragment including two Fab′ fragments linked by a disulfide bridge between the heavy chains at the hinge region.

The “Fv” fragment is the minimum fragment that contains a complete antigen recognition and binding site from an antibody. This fragment consists of a dimer of one immunoglobulin heavy chain variable region (VH) and one immunoglobulin light chain variable region (VL) in tight, non-covalent association. It is in this configuration that the three CDRs of each variable region interact to define an antigen binding site on the surface of the VH-VL dimer. A single light chain or heavy chain variable region (or half of an Fv fragment comprising only three CDRs specific for an antigen) has the ability to recognize and bind antigen, although at a lower affinity than the entire binding site comprising both VH and VL.

A “single-chain variable fragment” or “scFv fragment” comprises the VH and VL regions of an antibody, wherein these regions are present in a single polypeptide chain, and optionally comprising a peptide linker between the VH and VL regions that enables the Fv to form the desired structure for antigen binding (see e.g., Bird et al., Science, Vol. 242:423-426, 1988; and Huston et al., Proc. Natl. Acad. Sci. USA, Vol. 85:5879-5883, 1988).

A “nanobody” is the heavy chain variable region of a heavy-chain antibody. Such variable domains are the smallest fully functional antigen-binding fragment of such heavy-chain antibodies with a molecular mass of only 15 kDa. See Cortez-Retamozo et al., Cancer Research 64:2853-57, 2004. Functional heavy-chain antibodies devoid of light chains are naturally occurring in certain species of animals, such as nurse sharks, wobbegong sharks, and Camelidae, such as camels, dromedaries, alpacas and llamas. The antigen-binding site is reduced to a single domain, the VHH domain, in these animals. These antibodies form antigen-binding regions using only heavy chain variable region, i.e., these functional antibodies are homodimers of heavy chains only having the structure H2L2 (referred to as “heavy-chain antibodies” or “HCAbs”). Camelized VHH reportedly recombines with IgG2 and IgG3 constant regions that contain hinge, CH2, and CH3 domains and lack a CH1 domain. Camelized VHH domains have been found to bind to antigen with high affinity (Desmyter et al., J. Biol. Chem., Vol. 276:26285-90, 2001) and possess high stability in solution (Ewert et al., Biochemistry, Vol. 41:3628-36, 2002). Methods for generating antibodies having camelized heavy chains are described in, for example, U.S. Patent Publication Nos. 2005/0136049 and 2005/0037421. Alternative scaffolds can be made from human variable-like domains that more closely match the shark V-NAR scaffold and may provide a framework for a long penetrating loop structure.

As used herein, the term “antigen binding protein” refers to a protein that specifically binds to one or more target antigens. An antigen binding protein typically comprises an antigen-binding fragment that specifically binds to an antigen and, optionally, a scaffold or framework portion that allows the antigen-binding fragment to adopt a conformation that promotes binding of the antigen binding protein to the antigen. In some embodiments, an antigen binding protein is an antibody or antibody fragment. In some embodiments, an antigen binding protein is a protein comprising one or more antigen-binding fragments incorporated into a single polypeptide chain or into multiple polypeptide chains. For instance, antigen binding proteins can include, but are not limited to, a diabody (see, e.g., EP 404,097; WO 93/11161; and HoUinger et al., Proc. Natl. Acad. Sci. USA, Vol. 90:6444-6448, 1993); an intrabody; a domain antibody (single VL or VH domain or two or more VH domains joined by a peptide linker; see Ward et al, Nature, Vol. 341:544-546, 1989); a maxibody (2 scFvs fused to Fc region, see Fredericks et al, Protein Engineering, Design & Selection, Vol. 17:95-106, 2004 and Powers et al., Journal of Immunological Methods, Vol. 251:123-135, 2001); a triabody; a tetrabody; a minibody (scFv fused to CH3 domain; see Olafsen et al, Protein Eng Des Sel., Vol. 17:315-23, 2004); a peptibody (one or more peptides attached to an Fc region, see WO 00/24782); a linear antibody (a pair of tandem Fd segments (VH-CH1-VH-CH1) which, together with complementary light chain polypeptides, form a pair of antigen binding regions, see Zzjpate et al., Protein Eng., Vol. 8: 1057-1062, 1995); a small modular immunopharmaceutical (see U.S. Patent Publication No. 20030133939); and immunoglobulin fusion proteins (e.g. IgG-scFv, IgG-Fab, 2scFv-IgG, 4scFv-IgG, VH-IgG, IgG-VH, and Fab-scFv-Fc; see, e.g., Spiess et al, Mol. Immunol., Vol. 67(2 Pt A):95-106, 2015).

As used herein, a “c-Kit binding protein” is an antigen binding protein that specifically binds to c-Kit. In some embodiments, a c-Kit binding protein may also bind to one or more target antigens other than c-Kit.

Antibodies and antibody fragments (such as, e.g., heavy chain-only antibodies and three-chain antibody like molecules) of the present disclosure include multi-specific antibodies and antibody fragments, which are antibodies and antibody fragments having more than one binding specificity. As used herein, the term “multi-specific” includes “bispecific” (i.e., two binding specificities) and “trispecific” (i.e., three binding specificities), as well as higher-order independent specific binding affinities, such as higher-order polyepitopic specificity.

As used herein, an “isolated” molecule (such as, e.g., an antibody, antibody fragment, single domain antibody, c-Kit binding protein) is a molecule which has been identified and separated and/or recovered from a component of its natural environment. Contaminant components of its natural environment are materials which may interfere with diagnostic or therapeutic uses for the molecule, such as, e.g., enzymes, hormones, and other proteinaceous or nonproteinaceous solutes. In some embodiments, the isolated molecule will be purified (1) to greater than 95% by weight of the molecule as determined by the Lowry method, such as, e.g., more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, e.g., silver stain. In some embodiments, an isolated molecule will be prepared by a process comprising at least one purification step.

As used herein, an “antibody-drug conjugate” refers to an antibody or antibody fragment which is coupled to another moiety, such as, e.g., a payload, such as, e.g., a radionuclide.

As used herein, an “epitope” is a site on the surface of an antigen molecule to which a single antibody or antibody fragment binds. Generally, an antigen has several or many different epitopes and reacts with many different antibodies and antibody fragments. The term specifically includes linear epitopes and conformational epitopes. Conformational and nonconformational epitopes are distinguished in that the binding to the former but not the latter is lost in the presence of denaturing solvents. The epitope may comprise amino acid residues directly involved in the binding (also called immunodominant component of the epitope) and other amino acid residues, which are not directly involved in the binding, such as, e.g., amino acid residues which are effectively blocked by the specifically antigen binding peptide (in other words, the amino acid residue is within the footprint of the specifically antigen binding peptide).

As used herein, “polyepitopic specificity” refers to the ability to specifically bind to two or more different epitopes on the same or different target(s).

The terms “subject,” “individual,” and “patient” are used interchangeably herein to refer to a mammal being assessed for treatment and/or being treated. Subjects may be human, but also include other mammals, such as, e.g., those mammals useful as laboratory models for human disease, such as, e.g., mouse, rat, etc. In some embodiments, the mammal is a human.

As used herein, the term “treatment” encompasses any improvement of a disease in the subject, including the slowing or stopping of the progression of a disease in the subject, a decrease in the number or severity of the symptoms of the disease, or an increase in frequency or duration of periods where the patient is free from the symptoms of the disease.

As used herein, the term “effector cell” refers to an immune cell which is involved in the effector phase of an immune response, as opposed to the cognitive and activation phases of an immune response. Example immune cells include a cell of a myeloid or lymphoid origin, for instance, lymphocytes (such as B cells and T cells including cytolytic T cells (CTLs)), killer cells, natural killer cells, macrophages, monocytes, eosinophils, polymorphonuclear cells, such as neutrophils, granulocytes, mast cells, and basophils. Some effector cells express specific Fc receptors (FcRs) and carry out specific immune functions. In some embodiments, an effector cell is capable of inducing ADCC, such as a natural killer cell. For example, monocytes, macrophages, which express FcRs are involved in specific killing of target cells and presenting antigens to other components of the immune system, or binding to cells that present antigens.

As used herein, the term “vector” refers to a nucleic acid molecule capable of transporting another nucleic acid to which it has been linked. One type of vector is a “plasmid,” which refers to a circular double-stranded DNA loop into which additional DNA segments may be ligated. Another type of vector is a viral vector, wherein additional DNA segments may be ligated into the viral genome. Certain vectors are capable of autonomous replication in a host cell into which they are introduced (such as, e.g., bacterial vectors having a bacterial origin of replication and episomal mammalian vectors). Other vectors (such as, e.g., non-episomal mammalian vectors) may be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors.” In some embodiments, expression vectors for use in recombinant DNA techniques are in the form of plasmids.

As used herein, the term “host cell” refers to a cell into which an expression vector has been introduced. It should be understood that “host cell” is intended to refer not only to the particular subject cell, but also to the progeny of such a cell. Because certain modifications may occur in succeeding generations due to either mutation or environmental influences, such progeny may not, in fact, be identical to the parent cell, but are still included within the scope of the term “host cell” as used herein. Example recombinant host cells include, but are not limited to, transfectomas, such as CHO cells, HEK293 cells, NS/0 cells, and lymphocytic cells.

The term “K_{D}” (M), as used herein, refers to the dissociation equilibrium constant of a particular antigen binding interaction as determined by BioLayer Interferometry, using an Octet QK384 instrument (Fortebio Inc., Menlo Park, CA) in kinetics mode. For example, anti-mouse Fc sensors are loaded with mouse-Fc fused antigen and then dipped into antibody-containing wells to measure concentration dependent association rates (kₒₙ). Antibody dissociation rates (k_{off}) are measured in the final step, where the sensors are dipped into wells containing buffer only. The K_{D} is the ratio of k_{off}/kₒₙ. (For further details see, Concepcion, J, et al., Comb Chem High Throughput Screen, 12(8), 791-800, 2009).

As used herein, a molecule (such as, e.g., a protein, antibody, or antibody fragment) “specifically binds” to a target antigen when it has a significantly higher binding affinity for, and consequently is capable of distinguishing, that antigen compared to its affinity for other unrelated proteins, under similar binding assay conditions. Molecules that specifically bind an antigen may bind to that antigen with an equilibrium dissociation constant (K_{D})≤1×10⁻⁶ M. Molecules specifically bind antigen with “high affinity” when the K_{D} is ≤1×10⁻⁸ M.

In some embodiments, molecules described herein bind to human c-Kit and/or human CD3 with a K_{D} of ≤5×10⁻⁷ M. In some embodiments, molecules described herein bind to human c-Kit and/or human CD3 with a K_{D} of ≤1×10⁻⁷ M. In some embodiments, molecules described herein bind to human c-Kit and/or human CD3 with a K_{D} of ≤5×10⁻⁸ M. In some embodiments, molecules described herein bind to human c-Kit and/or human CD3 with a K_{D} of ≤2×10⁻⁸ M. In some embodiments, molecules described herein bind to human c-Kit and/or human CD3 with a K_{D} of ≤1×10⁻⁸ M. In some embodiments, molecules described herein bind to human c-Kit and/or human CD3 with a K_{D} of ≤1×10⁻⁹ M.

In some embodiments, molecules described herein bind to a GNNK+ isoform of human c-Kit and/or human CD3 with a K_{D} of ≤5×10⁻⁷ M. In some embodiments, molecules described herein bind to a GNNK+ isoform of human c-Kit and/or human CD3 with a K_{D} of ≤1×10⁻⁷ M. In some embodiments, molecules described herein bind to a GNNK+ isoform of human c-Kit and/or human CD3 with a K_{D} of ≤5×10⁻⁸ M. In some embodiments, molecules described herein bind to a GNNK+ isoform of human c-Kit and/or human CD3 with a K_{D} of ≤2×10⁻⁸ M. In some embodiments, molecules described herein bind to a GNNK+ isoform of human c-Kit and/or human CD3 with a K_{D} of ≤1×10⁻⁸ M. In some embodiments, molecules described herein bind to a GNNK+ isoform of human c-Kit and/or human CD3 with a K_{D} of ≤1×10⁻⁹ M.

In some embodiments, molecules described herein bind to a GNNK− isoform of human c-Kit and/or human CD3 with a K_{D} of ≤5×10⁻⁷ M. In some embodiments, molecules described herein bind to a GNNK− isoform of human c-Kit and/or human CD3 with a K_{D} of ≤1×10⁻⁷ M. In some embodiments, molecules described herein bind to a GNNK− isoform of human c-Kit and/or human CD3 with a K_{D} of ≤5×10⁻⁸ M. In some embodiments, molecules described herein bind to a GNNK− isoform of human c-Kit and/or human CD3 with a K_{D} of ≤2×10⁻⁸ M. In some embodiments, molecules described herein bind to a GNNK− isoform of human c-Kit and/or human CD3 with a K_{D} of ≤1×10⁻⁸ M. In some embodiments, molecules described herein bind to a GNNK− isoform of human c-Kit and/or human CD3 with a K_{D} of ≤1×10⁻⁹ M.

Affinity may be determined using a variety of techniques, a non-limiting example of which is an affinity ELISA assay. In some embodiments, affinity is determined by a surface plasmon resonance assay (e.g., BIAcore®-based assay). Using this methodology, the association rate constant (kₐ in M⁻¹s⁻¹) and the dissociation rate constant (k_{d} in s⁻¹) can be measured. The equilibrium dissociation constant (K_{D} in M) can then be calculated from the ratio of the kinetic rate constants (k_{d}/kₐ). In some embodiments, affinity is determined by a kinetic method, such as a Kinetic Exclusion Assay (KinExA) as described in Rathanaswami et al. Analytical Biochemistry, Vol. 373:52-60, 2008. Using a KinExA assay, the equilibrium dissociation constant (K_{D} in M) and the association rate constant (ka in M⁻¹s⁻¹) can be measured. The dissociation rate constant (k_{d} in s⁻¹) can be calculated from these values (K_{D}×kₐ). In other embodiments, affinity is determined by a bio-layer interferometry method, such as that described in Kumaraswamy et al., Methods Mol. Biol., Vol. 1278:165-82, 2015 and employed in Octet® systems (Pall ForteBio). The kinetic (kₐ and k_{d}) and affinity (K_{D}) constants can be calculated in real-time using the bio-layer interferometry method.

As used herein, an “[Y]-binding VH CDR” refers to a CDR of a VH region, wherein the VH region specifically binds to the target [Y].

As used herein, the term “amino acid” or “amino acid residue” refers to an amino acid having its art recognized definition, such as, e.g., an amino acid selected from the group consisting of: alanine (Ala or A); arginine (Arg or R); asparagine (Asn or N); aspartic acid (Asp or D); cysteine (Cys or C); glutamine (Gln or Q); glutamic acid (Glu or E); glycine (Gly or G); histidine (His or H); isoleucine (He or I): leucine (Leu or L); lysine (Lys or K); methionine (Met or M); phenylalanine (Phe or F); pro line (Pro or P); serine (Ser or S); threonine (Thr or T); tryptophan (Trp or W); tyrosine (Tyr or Y); and valine (Val or V), although modified, synthetic, or rare amino acids may be used as desired. Generally, amino acids can be grouped as having a nonpolar side chain (e.g., Ala, Cys, He, Leu, Met, Phe, Pro, Val); a negatively charged side chain (e.g., Asp, Glu); a positively charged sidechain (e.g., Arg, His, Lys); or an uncharged polar side chain (e.g., Asn, Cys, Gln, Gly, His, Met, Phe, Ser, Thr, Trp, and Tyr).

As used herein, “amino acid modifications” include, but are not limited to, deletions from, and/or insertions into, and/or substitutions of, residues within an amino acid sequence. Any combination of deletion, insertion, and substitution may be made to arrive at a final construct, provided that the final construct possesses the desired characteristics. The amino acid changes also may alter post-translational processes of the antibody constructs, such as changing the number or position of glycosylation sites. Preferred substitutions (or replacements) are conservative substitutions. However, any substitution (including non-conservative substitutions) is envisaged as long as the final construct retains its capability to bind to the target antigen.

One of skill in the art will realize that conservative variants of the antibodies and antibody fragments described herein can be produced. Such conservative variants employed in antibody fragments, such as dsFv fragments or in scFv fragments, will retain critical amino acid residues necessary for correct folding and stabilizing between the V_{H} and the V_{L} regions, and will retain the charge characteristics of the residues in order to preserve the low pI and low toxicity of the molecules. In some embodiments, amino acid substitutions (such as, e.g., at most one, at most two, at most three, at most four, or at most five amino acid substitutions) can be made in the VH and/or the VL regions to increase yield. Conservative amino acid substitution tables providing functionally similar amino acids are well known to one of ordinary skill in the art, such as, e.g., those described in Table A1.

TABLE A1
Example Conservative Substitutions

| | | Specific Example |
|---|---|---|
| Original | Example Substitutions | Substitutions |
| Ala (A) | Val, Leu, Ile | Val |
| Arg (R) | Lys, Gln, Asn | Lys |
| Asn (N) | Gln, His, Asp, Lys, Arg | Gln |
| Asp (D) | Glu, Asn | Glu |
| Cys (C) | Ser, Ala | Ser |
| Gln (Q) | Asn, Glu | Asn |
| Glu (E) | Asp, Gln | Asp |
| Gly (G) | Ala | Ala |
| His (H) | Asn, Gln, Lys, Arg | Arg |
| Ile (I) | Leu, Val, Met, Ala, Phe | Leu |
| Leu (L) | Norleucine, Ile, Val, Met, Ala | Ile |
| Lys (K) | Arg, Gln, Asn | Arg |
| Met (M) | Leu, Phe, Ile | Leu |
| Phe (F) | Leu, Val, Ile, Ala, Tyr | Tyr |
| Pro (P) | Ala | Ala |
| Ser (S) | Thr | Thr |
| Thr (T) | Ser | Ser |
| Trp (W) | Tyr, Phe | Tyr |
| Tyr (Y) | Trp, Phe, Thr, Ser | Phe |
| Val (V) | Ile, Leu, Met, Phe, Ala | Leu |

As used herein, the term “chimeric antigen receptor,” or alternatively “CAR,” refers to a recombinant polypeptide construct comprising at least an extracellular antigen binding domain, a transmembrane domain, and a cytoplasmic signaling domain (also referred to herein as “an intracellular signaling domain”) comprising a functional signaling domain derived from a stimulatory molecule. In some embodiments, the domains in the CAR polypeptide construct are in the same polypeptide chain, e.g., comprise a chimeric fusion protein. In some embodiments, the domains in the CAR polypeptide construct are not contiguous with each other, e.g., are in different polypeptide chains.

In some embodiments, the stimulatory molecule is the zeta chain associated with the I-cell receptor complex. In some embodiments, the cytoplasmic signaling domain comprises a primary signaling domain (e.g., a primary signaling domain of CD3-zeta). In some embodiments, the cytoplasmic signaling domain further comprises one or more functional signaling domains derived from at least one costimulatory molecule. In some embodiments, the costimulatory molecule is chosen from 4-1BB (i.e., CD137), CD27, ICOS, and CD28. In some embodiments, the CAR comprises a chimeric fusion protein comprising an extracellular antigen binding domain, a transmembrane domain, and an intracellular signaling domain comprising a functional signaling domain derived from a stimulatory molecule. In some embodiments, the CAR comprises a chimeric fusion protein comprising an extracellular antigen binding domain, a transmembrane domain, and an intracellular signaling domain comprising a functional signaling domain derived from a co-stimulatory molecule and a functional signaling domain derived from a stimulatory molecule. In some embodiments, the CAR comprises a chimeric fusion protein comprising an extracellular antigen binding domain, a transmembrane domain, and an intracellular signaling domain comprising two functional signaling domains derived from one or more co-stimulatory molecule(s) and a functional signaling domain derived from a stimulatory molecule. In some embodiments, the CAR comprises a chimeric fusion protein comprising an extracellular antigen binding domain, a transmembrane domain, and an intracellular signaling domain comprising at least two functional signaling domains derived from one or more co-stimulatory molecule(s) and a functional signaling domain derived from a stimulatory molecule. In some embodiments, the CAR comprises an optional leader sequence at the amino-terminus (N-ter) of the CAR fusion protein. In some embodiments, the CAR further comprises a leader sequence at the N-terminus of the extracellular antigen binding domain, wherein the leader sequence is optionally cleaved from the antigen recognition domain (e.g., a scFv) during cellular processing and localization of the CAR to the cellular membrane.

In some embodiments, the CAR further comprises a hinge between the extracellular antigen binding domain and the transmembrane domain. In some embodiments, the hinge comprises a sequence derived from a human CD8a, IgG4, and/or CD4 sequence. In some embodiments, the hinge comprises a sequence derived from a human CD8a sequence. In some embodiments, the hinge comprises an amino acid sequence comprising TTTPAPRPPTPAPTIASQPLSLRPEACRPAAGGAVHTRGLDFACD (SEQ ID NO: 72) or a sequence having at least about 70%, at least about 80%, at least about 90%, at least about 95%, or at least about 99% identity to the amino acid sequence comprising SEQ ID NO: 72. In some embodiments, the hinge is encoded by a nucleic acid sequence comprising actaccacaccagcacctagaccaccaactccagctccaaccatcgcgagtcagcccctgagtctgagacctgaggcctgcaggcc agctgcaggaggagctgtgcacaccaggggcctggacttcgcctgcgac (SEQ ID NO: 73). In some embodiments the hinge is encoded b a nucleic acid sequence comprising

(SEQ ID NO: 74)
ACCACAACCCCTGCCCCCAGACCTCCCACACCCGCCCCTACCATCGCGA
GTCAGCCCCTGAGTCTGAGACCTGAGGCCTGCAGGCCAGCTGCAGGAGG
AGCTGTGCACACCAGGGGCCTGGACTTCGCCTGCGAC.

As used herein, the term “signaling domain” refers to the functional portion of a protein which acts by transmitting information within the cell to regulate cellular activity via defined signaling pathways by generating second messengers or functioning as effectors by responding to such messengers. In some embodiments, the signaling domain of a CAR described herein is derived from a stimulatory molecule or co-stimulatory molecule, or is a synthesized or engineered signaling domain.

As used herein, an “intracellular signaling domain” refers to an intracellular portion of a molecule. The intracellular signaling domain generates a signal that promotes an immune effector function of the CAR-expressing cell, e.g., a CAR-T cell or CAR-expressing NK cell. Non-limiting examples of immune effector function, e.g., in a CAR-T cell or CAR-expressing NK cell, include cytolytic activity and helper activity, including the secretion of cytokines. While the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal.

In some embodiments, the intracellular signaling domain may comprise a primary intracellular signaling domain. Example primary intracellular signaling domains include, but are not limited to, those derived from the molecules responsible for primary stimulation, or antigen dependent simulation. In some embodiments, the intracellular signaling domain comprises a costimulatory intracellular domain. Example costimulatory intracellular signaling domains include, but are not limited to, those derived from molecules responsible for costimulatory signals, or antigen independent stimulation. In some embodiments, the intracellular signaling domain is synthesized or engineered. For example, in the case of a CAR-expressing immune effector cell, e.g., a CAR-T cell or CAR-expressing NK cell, a primary intracellular signaling domain may comprise a cytoplasmic sequence of a T cell receptor, a primary intracellular signaling domain may comprise a cytoplasmic sequence of a T cell receptor, and a costimulatory intracellular signaling domain may comprise cytoplasmic sequence from co-receptor or costimulatory molecule.

In some embodiments, a primary intracellular signaling domain comprises a signaling motif which is known as an immunoreceptor tyrosine-based activation motif or ITAM. Examples of ITAM containing primary cytoplasmic signaling sequences include, but are not limited to, those derived from CD3 zeta, common FcR gamma (FCER1G), Fc gamma RIIa, FcR beta, CD3 gamma, CD3 delta, CD3 epsilon, CDS, CD22, CD79a, CD79b, CD278 (“ICOS”), FcεRI CD66d, DAP10 and DAP12.

As used herein, the term “costimulatory molecule” refers to the cognate binding partner on a T cell that specifically binds with a costimulatory ligand, thereby mediating a costimulatory response by the T cell, including, but not limited to, proliferation. Costimulatory molecules are cell surface molecules other than antigen receptors or their ligands that are required for an efficient immune response. Costimulatory molecules include, but are not limited to, an MHC class I molecule, a TNF receptor protein, an Immunoglobulin-like protein, a cytokine receptor, an integrin, a signaling lymphocytic activation molecule (SLAM protein), an activating NK cell receptor, BTLA, a Toll ligand receptor, OX40, CD2, CD7, CD27, CD28, CD30, CD40, CDS, ICAM-1, LFA-1 (CD11a/CD18), 4-1BB (CD137), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83.

In some embodiments, a costimulatory intracellular signaling domain can be the intracellular portion of a costimulatory molecule. The intracellular signaling domain can comprise the entire intracellular portion, or the entire native intracellular signaling domain, of the molecule from which it is derived, or a functional fragment thereof

As used herein, the term “zeta,” or alternatively “zeta chain” or “CD3-zeta,” is defined as the protein provided as GenBan Acc. No. BAG36664.1, or the equivalent residues from a non-human species, e.g., mouse, rodent, monkey, ape, and the like, and a “zeta stimulatory domain,” or alternatively a “CD3-zeta stimulatory domain,” is defined as the amino acid residues from the cytoplasmic domain of the zeta chain that are sufficient to functionally transmit an initial signal necessary for T cell activation. In some embodiments, the cytoplasmic domain of zeta comprises residues 52 through 164 of GenBank Acc. No. BAG36664.1 or the equivalent residues from a non-human species, e.g., mouse, rodent, monkey, ape and the like, that are functional orthologs thereof.

As used herein, the term “4-1BB” refers to a member of the TNFR superfamily with an amino acid sequence provided as GenBank Acc. No. AAA62478.2, or the equivalent residues from a non-human species, e.g., mouse, rodent, monkey, ape and the like; and a “4-1BB costimulatory domain” is defined as amino acid residues 214-255 of GenBank Acc. No. AAA62478.2, or the equivalent residues from a non-human species, e.g., mouse, rodent, monkey, ape and the like.

As used herein, “percent (%) amino acid sequence identity” or “percent (%) sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2.

The term “pharmaceutical composition” refers to a preparation which is in such form as to permit the biological activity of the active ingredient to be effective, and which contains no additional components which are unacceptably toxic to a subject to which the formulation would be administered. Such compositions are sterile. “Pharmaceutically acceptable” excipients (e.g., vehicles, additives) are those which can reasonably be administered to a subject mammal to provide an effective dose of the active ingredient employed.

As used herein, a “sterile” composition is aseptic or free or essentially free from all living microorganisms and their spores. As used herein, a “frozen” composition is one at a temperature below 0° C.

As used herein, a “stable” composition is one in which the protein therein essentially retains its physical stability and/or chemical stability and/or biological activity upon storage. In some embodiments, the composition essentially retains its physical and chemical stability, as well as its biological activity upon storage. The storage period is generally selected based on the intended shelf-life of the composition. Various analytical techniques for measuring protein stability are available in the art and are reviewed in Peptide and Protein Drug Delivery, 247-301. Vincent Lee Ed., Marcel Dekker, Inc., New York, N.Y., Pubs. (1991) and Jones. A. Adv. Drug Delivery Rev. 10: 29-90) (1993), for example. Stability can be measured at a selected temperature for a selected time period. Stability can be evaluated qualitatively and/or quantitatively in a variety of different ways, including evaluation of aggregate formation (for example, using size exclusion chromatography, by measuring turbidity, and/or by visual inspection); by assessing charge heterogeneity using cation exchange chromatography, image capillary isoelectric focusing (icIEF) or capillary zone electrophoresis; amino-terminal or carboxy-terminal sequence analysis; mass spectrometric analysis; SDS-PAGE analysis to compare reduced and intact antibody; peptide map (for example tryptic or LYS-C) analysis; evaluating biological activity or antigen binding function of the antibody; etc. Instability may involve any one or more of: aggregation, deamidation (e.g., Asn deamidation), oxidation (e.g., Met oxidation), isomerization (e.g., Asp isomerization), clipping/hydrolysis/fragmentation (e.g., hinge region fragmentation), succinimide formation, unpaired cysteine(s), N-terminal extension, C-terminal processing, glycosylation differences, etc.

Some embodiments of the present disclosure relate to a single domain antibody which specifically binds to c-Kit. Specifically, the present disclosure provides a family of closely related single domain antibodies that specifically bind to human c-Kit. The single domain antibodies of this family comprise a set of CDR sequences as defined herein and as shown in Tables S1 and S2, and are exemplified by the provided heavy chain variable region (VH) sequences of SEQ ID NOs: 14-17 as set forth in Table S4. This family of single domain antibodies provides a number of benefits that contribute to their utility as clinically therapeutic agent(s). Illustratively, the single domain antibodies include members with a range of binding affinities, allowing the selection of a specific sequence with a desired binding affinity. Additionally, the present disclosure provides a single domain antibody comprising a set of CDR sequences as shown in Table S3 and as exemplified by the VH sequence of SEQ ID NO: 18 as set forth in Table S4.

TABLE S1
F06 Family Anti-c-Kit Heavy Chain Antibody CDR1, CDR2 and CDR3
Amino Acid Sequences

| Clone | | | |
|---|---|---|---|
| ID # | SEQ_aa_CDR1 | SEQ_aa_CDR2 | SEQ_aa_CDR3 |
| 408366 | GLTISTYA | ISTGGSST | ATGYDSSGHYYGGFDY |
| | (SEQ ID NO: 1) | (SEQ ID NO: 4) | (SEQ ID NO: 8) |
| 407921 | GFTFDSYA | ISVRGGST | ATGYDPSGHYYGGFDY |
| | (SEQ ID NO: 2) | (SEQ ID NO: 5) | (SEQ ID NO: 9) |
| 407922 | GLTISSYA | ISTGGSRT | ATGYDSSGHYYGGFDY |
| | (SEQ ID NO: 3) | (SEQ ID NO: 6) | (SEQ ID NO: 8) |
| 408389 | GFTFDSYA | ISTGGGST | ATGYDSSGYYYGGFDY |
| | (SEQ ID NO: 2) | (SEQ ID NO: 7) | (SEQ ID NO: 10) |

TABLE S2
F06 Family Anti-c-Kit Heavy Chain Antibody Unique CDR
Amino Acid Sequences

| SEQ_aa_CDR1 | SEQ_aa_CDR2 | SEQ_aa_CDR3 |
|---|---|---|
| GLTISTYA | ISTGGSST | ATGYDSSGHYYGGFDY |
| (SEQ ID NO: 1) | (SEQ ID NO: 4) | (SEQ ID NO: 8) |
| GFTFDSYA | ISVRGGST | ATGYDPSGHYYGGFDY |
| (SEQ ID NO: 2) | (SEQ ID NO: 5) | (SEQ ID NO: 9) |
| GLTISSYA | ISTGGSRT | ATGYDSSGYYYGGFDY |
| (SEQ ID NO: 3) | (SEQ ID NO: 6) | (SEQ ID NO: 10) |
| | ISTGGGST | |
| | (SEQ ID NO: 7) | |

TABLE S3
F18 Family Anti-c-Kit Heavy Chain Antibody CDR1, CDR2 and CDR3
Amino Acid Sequences

| Clone | | | |
|---|---|---|---|
| ID # | SEQ_aa_CDR1 | SEQ_aa_CDR2 | SEQ_aa_CDR3 |
| 408198 | GGSISSSSYY | IDYNGGT | ARQNPRRYASGASDY |
| | (SEQ ID NO: 11) | (SEQ ID NO: 12) | (SEQ ID NO: 13) |

TABLE S4
Anti-c-Kit Heavy Chain Antibody Variable Domain Amino Acid Sequences

| Clone | | SEQ ID |
|---|---|---|
| ID # | SEQ_aa_FR1_FR4 | NO. |
| 408366 | EVQLLESGGGLVQPGGSLRLSCAASGLTISTYAMSWVRQAPGK | 14 |
| | GLEWVSAISTGGSSTYYADSVKGRFTISRDNSKNTLYLQMNSLR | |
| | AEDTAVYYCATGYDSSGHYYGGFDYRGQGTLVTVSS | |
| 407921 | EVQLLESGGGLVQPGGSLRLSCAASGFTFDSYAMSWVRQAPGK | 15 |
| | GLEWVSAISVRGGSTYYADSVKGRFTISRDNSKNTLYLQMNSLR | |
| | AEDTAVYSCATGYDPSGHYYGGFDYRGQGTLVTVSS | |
| 407922 | EVQLLESGGGLVQPGGSLRLSCAASGLTISSYAMSWVRQAPGKG | 16 |
| | LEWVSAISTGGSRTYYADSVKGRFTISRDNSKNTLYLQMNSLRA | |
| | EDTAVYFCATGYDSSGHYYGGFDYRGQGTLVTVSS | |
| 408389 | EVQLLESGGGLVQPGGSLRLSCAASGFTFDSYAMSWVRQAPGE | 17 |
| | GLEWVSAISTGGGSTYYADSVKGRFTISRDNSKNMLFLQMNSLR | |
| | AEDTAVYSCATGYDSSGYYYGGFDYRGQGTLVTVSS | |
| 408198 | QLQLQESGPGLVKPSETLSLTCTVSGGSISSSSYYWGWIRQPPGK | 18 |
| | GLEWIGSIDYNGGTYNNPSLKSRVTISVDTSKNQFSLKLSYVTAA | |
| | DTAVYYCARQNPRRYASGASDYRGQGTLVTVSS | |

In some embodiments, the single domain antibody comprises a heavy chain variable (VH) region comprising:
(i) a VH complementarity determining region one (CDR1) comprising the sequence

(SEQ ID NO: 53)
G X₁ T X₂ X₃ X₄ Y A,

wherein X₁ is F or L; X₂ is F or I; X₃ is D or S; and X₄ is S or T;
(ii) a VH CDR2 comprising the sequence

(SEQ ID NO: 71)
I S X₅ X₆ G X₇ X₈ T,

wherein X₅ is V or T, X₆ is R or G; X₇ is G or S; and X₈ is S or R; and
(iii) a VH CDR3 comprising the sequence

(SEQ ID NO: 54)
A T G Y D X₉ S G X₁₀ Y Y G G F D Y,

wherein X₉ is S or P; and X₁₀ is Y or H.

In some embodiments, the single domain antibody comprises a heavy chain variable (VH) region in which the full set of VH CDRs 1, 2, and 3 (combined) has at least 80% (such as, e.g., 80%, 85%, 90%, 95%, at least 85%, at least 90%, at least 95%) sequence identity to the VH CDRs 1, 2, and 3 of any one of SEQ ID NOs: 14-18. In some embodiments, the single domain antibody comprises a heavy chain variable (VH) region in which the full set of VH CDRs 1, 2, and 3 (combined) has at least 85% (such as, e.g., 85%, 90%, 95%, at least 90%, at least 95%) sequence identity to the VH CDRs 1, 2, and 3 of any one of SEQ ID NOs: 14-18. In some embodiments, the single domain antibody comprises a heavy chain variable (VH) region in which the full set of VH CDRs 1, 2, and 3 (combined) has at least 90% (such as, e.g., 90%, 95%, at least 95%) sequence identity to the VH CDRs 1, 2, and 3 of any one of SEQ ID NOs: 14-18. In some embodiments, the single domain antibody comprises a heavy chain variable (VH) region in which the full set of VH CDRs 1, 2, and 3 (combined) has at least 95% sequence identity to the VH CDRs 1, 2, and 3 of any one of SEQ ID NOs: 14-18.

In some embodiments, the single domain antibody comprises a heavy chain variable (VH) region in which the full set of VH CDRs 1, 2, and 3 (combined) has at least 80% (such as, e.g., 80%, 85%, 90%, 95%, at least 85%, at least 90%, at least 95%) sequence identity to the VH CDRs 1, 2, and 3 of SEQ ID NO: 14. In some embodiments, the single domain antibody comprises a heavy chain variable (VH) region in which the full set of VH CDRs 1, 2, and 3 (combined) has at least 80% (such as, e.g., 80%, 85%, 90%, 95%, at least 85%, at least 90%, at least 95%) sequence identity to the VH CDRs 1, 2, and 3 of SEQ ID NO: 15. In some embodiments, the single domain antibody comprises a heavy chain variable (VH) region in which the full set of VH CDRs 1, 2, and 3 (combined) has at least 80% (such as, e.g., 80%, 85%, 90%, 95%, at least 85%, at least 90%, at least 95%) sequence identity to the VH CDRs 1, 2, and 3 of SEQ ID NO: 16. In some embodiments, the single domain antibody comprises a heavy chain variable (VH) region in which the full set of VH CDRs 1, 2, and 3 (combined) has at least 80% (such as, e.g., 80%, 85%, 90%, 95%, at least 85%, at least 90%, at least 95%) sequence identity to the VH CDRs 1, 2, and 3 of SEQ ID NO: 17. In some embodiments, the single domain antibody comprises a heavy chain variable (VH) region in which the full set of VH CDRs 1, 2, and 3 (combined) has at least 80% (such as, e.g., 80%, 85%, 90%, 95%, at least 85%, at least 90%, at least 95%) sequence identity to the VH CDRs 1, 2, and 3 of SEQ ID NO: 18.

In some embodiments, the single domain antibody comprises a heavy chain variable (VH) region comprising:
(i) a VH complementarity determining region one (CDR1) comprising a sequence having at most two (e.g., one, two, zero) amino acid modifications relative to SEQ ID NO: 1, SEQ ID NO: 2, or SEQ ID NO: 3;
(ii) a VH CDR2 comprising a sequence having at most two (e.g., one, two, zero) amino acid modifications relative to SEQ ID NO: 4, SEQ ID NO: 5, SEQ ID NO: 6, or SEQ ID NO: 7; and
(iii) a VH CDR3 comprising a sequence having at most two (e.g., one, two, zero) amino acid modifications relative to SEQ ID NO: 8, SEQ ID NO: 9, or SEQ ID NO: 10.

In some embodiments, the single domain antibody comprises a heavy chain variable (VH) region comprising: (i) a VH CDR1 comprising a sequence having at most two (e.g., one, two, zero) amino acid modifications relative to SEQ ID NO: 1; (ii) a VH CDR2 comprising a sequence having at most two (e.g., one, two, zero) amino acid modifications relative to SEQ ID NO: 4; and (iii) a VH CDR3 comprising a sequence having at most two (e.g., one, two, zero) amino acid modifications relative to SEQ ID NO: 8.

In some embodiments, the single domain antibody comprises a heavy chain variable (VH) region comprising: (i) a VH CDR1 comprising a sequence having at most two (e.g., one, two, zero) amino acid modifications relative to SEQ ID NO: 2; (ii) a VH CDR2 comprising a sequence having at most two (e.g., one, two, zero) amino acid modifications relative to SEQ ID NO: 5; and (iii) a VH CDR3 comprising a sequence having at most two (e.g., one, two, zero) amino acid modifications relative to SEQ ID NO: 9.

In some embodiments, the single domain antibody comprises a heavy chain variable (VH) region comprising: (i) a VH CDR1 comprising a sequence having at most two (e.g., one, two, zero) amino acid modifications relative to SEQ ID NO: 3; (ii) a VH CDR2 comprising a sequence having at most two (e.g., one, two, zero) amino acid modifications relative to SEQ ID NO: 6; and (iii) a VH CDR3 comprising a sequence having at most two (e.g., one, two, zero) amino acid modifications relative to SEQ ID NO: 8.

In some embodiments, the single domain antibody comprises a heavy chain variable (VH) region comprising: (i) a VH CDR1 comprising a sequence having at most two (e.g., one, two, zero) amino acid modifications relative to SEQ ID NO: 2; (ii) a VH CDR2 comprising a sequence having at most two (e.g., one, two, zero) amino acid modifications relative to SEQ ID NO: 7; and (iii) a VH CDR3 comprising a sequence having at most two (e.g., one, two, zero) amino acid modifications relative to SEQ ID NO: 10.

In some embodiments, the single domain antibody comprises a heavy chain variable (VH) region comprising: (i) a VH CDR1 comprising a sequence having at most two (e.g., one, two, zero) amino acid modifications relative to SEQ ID NO: 11; (ii) a VH CDR2 comprising a sequence having at most two (e.g., one, two, zero) amino acid modifications relative to SEQ ID NO: 12; and (iii) a VH CDR3 comprising a sequence having at most two (e.g., one, two, zero) amino acid modifications relative to SEQ ID NO: 13.

In some embodiments, each amino acid modification, if any, is a conservative amino acid substitution. In some embodiments, each amino acid modification, if any, is a conservative amino acid substitution listed in Table A1.

In some embodiments, the VH CDR1 comprises a sequence having at most one amino acid modification relative to SEQ ID NO: 1, SEQ ID NO: 2, or SEQ ID NO: 3. In some embodiments, the VH CDR2 comprises a sequence having at most one amino acid modification relative to SEQ ID NO: 4, SEQ ID NO: 5, SEQ ID NO: 6, or SEQ ID NO: 7. In some embodiments, the VH CDR3 comprises a sequence having at most one amino acid modification relative to SEQ ID NO: 8, SEQ ID NO: 9, or SEQ ID NO: 10. In some embodiments, the at most one amino acid modification is an amino acid substitution. In some embodiments, the at most one amino acid modification is a conservative amino acid substitution. In some embodiments, the at most one amino acid modification is an amino acid deletion. In some embodiments, the at most one amino acid modification is an amino acid addition.

In some embodiments, the VH CDR1 comprises a sequence chosen from SEQ ID NO: 1, SEQ ID NO: 2, and SEQ ID NO: 3. In some embodiments, the VH CDR2 comprises a sequence chosen from SEQ ID NO: 4, SEQ ID NO: 5, SEQ ID NO: 6, and SEQ ID NO: 7. In some embodiments, the VH CDR3 comprises a sequence chosen from SEQ ID NO: 8, SEQ ID NO: 9, and SEQ ID NO: 10.

In some embodiments, the single domain antibody comprises a heavy chain variable (VH) region comprising:
(i) a VH complementarity determining region one (CDR1) comprising a sequence chosen from SEQ ID NO: 1, SEQ ID NO: 2, and SEQ ID NO: 3;
(ii) a VH CDR2 comprising a sequence chosen from SEQ ID NO: 4, SEQ ID NO: 5, SEQ ID NO: 6, and SEQ ID NO: 7; and
(iii) a VH CDR3 comprising a sequence chosen from SEQ ID NO: 8, SEQ ID NO: 9, and SEQ ID NO: 10.

In some embodiments, the single domain antibody comprises a heavy chain variable (VH) region comprising:
(a) a VH CDR1, a VH CDR2, and a VH CDR3 comprising the sequences of SEQ ID NOs: 1, 4, and 8, respectively;
(b) a VH CDR1, a VH CDR2, and a VH CDR3 comprising the sequences of SEQ ID NOs: 2, 5, and 9, respectively;
(c) a VH CDR1, a VH CDR2, and a VH CDR3 comprising the sequences of SEQ ID NOs: 3, 6, and 8, respectively;
(d) a VH CDR1, a VH CDR2, and a VH CDR3 comprising the sequences of SEQ ID NOs: 2, 7, and 10, respectively; or
(e) a VH CDR1, a VH CDR2, and a VH CDR3 comprising the sequences of SEQ ID NOs: 11, 12, and 13, respectively.

In some embodiments, the single domain antibody comprises a heavy chain variable (VH) region comprising a VH CDR1, a VH CDR2, and a VH CDR3 comprising the sequences of SEQ ID NOs: 1, 4, and 8, respectively. In some embodiments, the single domain antibody comprises a heavy chain variable (VH) region comprising a VH CDR1, a VH CDR2, and a VH CDR3 comprising the sequences of SEQ ID NOs: 2, 5, and 9, respectively. In some embodiments, the single domain antibody comprises a heavy chain variable (VH) region comprising a VH CDR1, a VH CDR2, and a VH CDR3 comprising the sequences of SEQ ID NOs: 3, 6, and 8, respectively. In some embodiments, the single domain antibody comprises a heavy chain variable (VH) region comprising a VH CDR1, a VH CDR2, and a VH CDR3 comprising the sequences of SEQ ID NOs: 2, 7, and 10, respectively. In some embodiments, the single domain antibody comprises a heavy chain variable (VH) region comprising a VH CDR1, a VH CDR2, and a VH CDR3 comprising the sequences of SEQ ID NOs: 11, 12, and 13, respectively.

In some embodiments, the single domain antibody comprises a heavy chain variable (VH) region comprising the CDR1, CDR2, and CDR3 of any one of SEQ ID NOs: 14-18. In some embodiments, the single domain antibody comprises a heavy chain variable (VH) region comprising the VH CDR1, VH CDR2, and VH CDR3 of SEQ ID NO: 14. In some embodiments, In some embodiments, the single domain antibody comprises a heavy chain variable (VH) region comprising the VH CDR1, VH CDR2, and VH CDR3 of SEQ ID NO: 15. In some embodiments, the single domain antibody comprises a heavy chain variable (VH) region comprising the VH CDR1, VH CDR2, and VH CDR3 of SEQ ID NO: 16. In some embodiments, the single domain antibody comprises a heavy chain variable (VH) region comprising the VH CDR1, VH CDR2, and VH CDR3 of SEQ ID NO: 17. In some embodiments, the single domain antibody comprises a heavy chain variable (VH) region comprising the VH CDR1, VH CDR2, and VH CDR3 of SEQ ID NO: 18.

In some embodiments, the VH CDR1, VH CDR2, and VH CDR3 sequences are present in a human VH framework.

In some embodiments, the single domain antibody comprises a heavy chain variable (VH) region having at least 80% (such as, e.g., 80%, 85%, 90%, 95%, 98%, 99%, at least 85%, at least 90%, at least 95%, at least 98%, at least 99%) sequence identity to any one of SEQ ID NOs: 14-18. In some embodiments, the single domain antibody comprises a heavy chain variable (VH) region having at least 85% (such as, e.g., 85%, 90%, 95%, 98%, 98%, 99%, at least 90%, at least 95%, at least 98%, at least 99%) sequence identity to any one of SEQ ID NOs: 14-18. In some embodiments, the single domain antibody comprises a heavy chain variable (VH) region having at least 90% (such as, e.g., 90%, 95%, 98%, 99%, at least 95%, at least 98%, at least 99%) sequence identity to any one of SEQ ID NOs: 14-18. In some embodiments, the single domain antibody comprises a heavy chain variable (VH) region having at least 95% sequence identity to any one of SEQ ID NOs: 14-18.

In some embodiments, the single domain antibody comprises a heavy chain variable (VH) region having at least 80% (such as, e.g., 80%, 85%, 90%, 95%, 98%, 99%, at least 85%, at least 90%, at least 95%, at least 98%, at least 99%) sequence identity to SEQ ID NO: 14. In some embodiments, the single domain antibody comprises a heavy chain variable (VH) region having at least 80% (such as, e.g., 80%, 85%, 90%, 95%, 89%, 99% at least 85%, at least 90%, at least 95%, at least 98%, at least 99%) sequence identity to SEQ ID NO: 15. In some embodiments, the single domain antibody comprises a heavy chain variable (VH) region having at least 80% (such as, e.g., 80%, 85%, 90%, 95%, 98%, 99%, at least 85%, at least 90%, at least 95%, at least 98%, at least 99%) sequence identity to SEQ ID NO: 16. In some embodiments, the single domain antibody comprises a heavy chain variable (VH) region having at least 80% (such as, e.g., 80%, 85%, 90%, 95%, 98%, 99%, at least 85%, at least 90%, at least 95%, at least 98%, at least 99%) sequence identity to SEQ ID NO: 17. In some embodiments, the single domain antibody comprises a heavy chain variable (VH) region having at least 80% (such as, e.g., 80%, 85%, 90%, 95%, 98%, 99%, at least 85%, at least 90%, at least 95%, at least 98%, at least 99%) sequence identity to SEQ ID NO: 18.

In some embodiments, the single domain antibody comprises a heavy chain variable (VH) region chosen from SEQ ID NOs: 14-18.

In some embodiments, the single domain antibody comprises the heavy chain variable (VH) region of SEQ ID NO: 14. In some embodiments, the single domain antibody comprises the heavy chain variable (VH) region of SEQ ID NO: 15. In some embodiments, the single domain antibody comprises the heavy chain variable (VH) region of SEQ ID NO: 16. In some embodiments, the single domain antibody comprises the heavy chain variable (VH) region of SEQ ID NO: 17. In some embodiments, the single domain antibody comprises the heavy chain variable (VH) region of SEQ ID NO: 18.

In some embodiments, the single domain antibody specifically binds to human c-Kit. In some embodiments, the single domain antibody binds to human c-Kit with a K_{D} of from about 10⁻⁹ M to about 10⁻⁶ M. In some embodiments, the single domain antibody binds to human c-Kit with a K_{D} of ≤5×10⁻⁷ M. In some embodiments, the single domain antibody binds to human c-Kit with a K_{D} of ≤1×10⁻⁷ M. In some embodiments, the single domain antibody binds to human c-Kit with a K_{D} of ≤5×10⁻⁸ M. In some embodiments, the single domain antibody binds to human c-Kit with a K_{D} of ≤2×10⁻⁸ M. In some embodiments, the single domain antibody binds to human c-Kit with a K_{D} of ≤1×10⁻⁸ M. In some embodiments, the single domain antibody binds to human c-Kit with a K_{D} of ≤1×10⁻⁹ M.

In some embodiments, the single domain antibody specifically binds to a GNNK− isoform of human c-Kit. In some embodiments, the single domain antibody binds to a GNNK− isoform of human c-Kit with a K_{D} of from about 10⁻⁹ M to about 10⁻⁶ M. In some embodiments, the single domain antibody binds to a GNNK− isoform of human c-Kit with a K_{D} of ≤5×10⁻⁷ M. In some embodiments, the single domain antibody binds to a GNNK− isoform of human c-Kit with a K_{D} of ≤1×10⁻⁷ M. In some embodiments, the single domain antibody binds to a GNNK− isoform of human c-Kit with a K_{D} of ≤5×10⁻⁸ M. In some embodiments, the single domain antibody binds to a GNNK− isoform of human c-Kit with a K_{D} of ≤2×10⁻⁸ M. In some embodiments, the single domain antibody binds to a GNNK− isoform of human c-Kit with a K_{D} of ≤1×10⁻⁸ M. In some embodiments, the single domain antibody binds to a GNNK− isoform of human c-Kit with a K_{D} of ≤1×10⁻⁹ M.

In some embodiments, the single domain antibody specifically binds to a GNNK+ isoform of human c-Kit. In some embodiments, the single domain antibody binds to a GNNK+ isoform of human c-Kit with a K_{D} of from about 10⁻⁹ M to about 10⁻⁶ M. In some embodiments, the single domain antibody binds to a GNNK+ isoform of human c-Kit with a K_{D} of ≤5×10⁻⁷ M. In some embodiments, the single domain antibody binds to a GNNK+ isoform of human c-Kit with a K_{D} of ≤1×10⁻⁷ M. In some embodiments, the single domain antibody binds to a GNNK+ isoform of human c-Kit with a K_{D} of ≤5×10⁻⁸ M. In some embodiments, the single domain antibody binds to a GNNK+ isoform of human c-Kit with a K_{D} of ≤2×10⁻⁸ M. In some embodiments, the single domain antibody binds to a GNNK+ isoform of human c-Kit with a K_{D} of ≤1×10⁻⁸ M. In some embodiments, the single domain antibody binds to a GNNK+ isoform of human c-Kit with a K_{D} of ≤1×10⁻⁹ M.

In some embodiments, the single domain antibody is a human single domain antibody.

In some embodiments, the single domain antibody is an isolated single domain antibody. In some embodiments, the single domain antibody is an isolated, human single domain antibody.

Some embodiments of the present disclosure relate to a c-Kit binding protein comprising a single domain antibody that specifically binds to c-Kit, as described herein.

In some embodiments, the single domain antibody comprises a heavy chain variable (VH) region comprising:
(i) a VH complementarity determining region one (CDR1) comprising the sequence

(SEQ ID NO: 53)
G X₁ T X₂ X₃ X₄ Y A,

wherein X₁ is F or L; X₂ is F or I; X₃ is D or S; and X₄ is S or T;
(ii) a VH CDR2 comprising the sequence

(SEQ ID NO: 71)
I S X₅ X₆ G X₇ X₈ T,

wherein X₅ is V or T, X₆ is R or G; X₇ is G or S; and X₈ is S or R; and
(iii) a VH CDR3 comprising the sequence

(SEQ ID NO: 54)
A T G Y D X₉ S G X₁₀ Y Y G G F D Y,

wherein X₉ is S or P; and X₁₀ is Y or H.

In some embodiments, the single domain antibody comprises a heavy chain variable (VH) region in which the full set of VH CDRs 1, 2, and 3 (combined) has at least 80% (such as, e.g., 80%, 85%, 90%, 95%, at least 85%, at least 90%, at least 95%) sequence identity to the VH CDRs 1, 2, and 3 of any one of SEQ ID NOs: 14-18. In some embodiments, the single domain antibody comprises a heavy chain variable (VH) region in which the full set of VH CDRs 1, 2, and 3 (combined) has at least 85% (such as, e.g., 85%, 90%, 95%, 98%, 99%, at least 90%, at least 95%, at least 98%, at least 99%) sequence identity to the VH CDRs 1, 2, and 3 of any one of SEQ ID NOs: 14-18. In some embodiments, the single domain antibody comprises a heavy chain variable (VH) region in which the full set of VH CDRs 1, 2, and 3 (combined) has at least 90% (such as, e.g., 90%, 95%, 98%, 99%, at least 95%, at least 98%, at least 99%) sequence identity to the VH CDRs 1, 2, and 3 of any one of SEQ ID NOs: 14-18. In some embodiments, the single domain antibody comprises a heavy chain variable (VH) region in which the full set of VH CDRs 1, 2, and 3 (combined) has at least 95% sequence identity to the VH CDRs 1, 2, and 3 of any one of SEQ ID NOs: 14-18.

In some embodiments, the single domain antibody comprises a heavy chain variable (VH) region in which the full set of VH CDRs 1, 2, and 3 (combined) has at least 80% (such as, e.g., 80%, 85%, 90%, 95%, 98%, 99%, at least 85%, at least 90%, at least 95%, at least 98%, at least 99%) sequence identity to the VH CDRs 1, 2, and 3 of SEQ ID NO: 14. In some embodiments, the single domain antibody comprises a heavy chain variable (VH) region in which the full set of VH CDRs 1, 2, and 3 (combined) has at least 80% (such as, e.g., 80%, 85%, 90%, 95%, 98%, 99%, at least 85%, at least 90%, at least 95%, at least 98%, at least 99%) sequence identity to the VH CDRs 1, 2, and 3 of SEQ ID NO: 15. In some embodiments, the single domain antibody comprises a heavy chain variable (VH) region in which the full set of VH CDRs 1, 2, and 3 (combined) has at least 80% (such as, e.g., 80%, 85%, 90%, 95%, 98% 99% at least 85%, at least 90%, at least 95%, at least 98%, at least 99%) sequence identity to the VH CDRs 1, 2, and 3 of SEQ ID NO: 16. In some embodiments, the single domain antibody comprises a heavy chain variable (VH) region in which the full set of VH CDRs 1, 2, and 3 (combined) has at least 80% (such as, e.g., 80%, 85%, 90%, 95%, 98%, 99%, at least 85%, at least 90%, at least 95%, at least 98%, at least 99%) sequence identity to the VH CDRs 1, 2, and 3 of SEQ ID NO: 17. In some embodiments, the single domain antibody comprises a heavy chain variable (VH) region in which the full set of VH CDRs 1, 2, and 3 (combined) has at least 80% (such as, e.g., 80%, 85%, 90%, 95%, 98%, 99%, at least 85%, at least 90%, at least 95%, at least 98%, at least 99%) sequence identity to the VH CDRs 1, 2, and 3 of SEQ ID NO: 18.

In some embodiments, the single domain antibody comprises a heavy chain variable (VH) region comprising:
(i) a VH complementarity determining region one (CDR1) comprising a sequence having at most two (e.g., one, two, zero) amino acid modifications relative to SEQ ID NO: 1, SEQ ID NO: 2, or SEQ ID NO: 3;
(ii) a VH CDR2 comprising a sequence having at most two (e.g., one, two, zero) amino acid modifications relative to SEQ ID NO: 4, SEQ ID NO: 5, SEQ ID NO: 6, or SEQ ID NO: 7; and
(iii) a VH CDR3 comprising a sequence having at most two (e.g., one, two, zero) amino acid modifications relative to SEQ ID NO: 8, SEQ ID NO: 9, or SEQ ID NO: 10.

In some embodiments, the single domain antibody comprises a heavy chain variable (VH) region comprising: (i) a VH CDR1 comprising a sequence having at most two (e.g., one, two, zero) amino acid modifications relative to SEQ ID NO: 1; (ii) a VH CDR2 comprising a sequence having at most two (e.g., one, two, zero) amino acid modifications relative to SEQ ID NO: 4; and (iii) a VH CDR3 comprising a sequence having at most two (e.g., one, two, zero) amino acid modifications relative to SEQ ID NO: 8.

In some embodiments, the single domain antibody comprises a heavy chain variable (VH) region comprising: (i) a VH CDR1 comprising a sequence having at most two (e.g., one, two, zero) amino acid modifications relative to SEQ ID NO: 2; (ii) a VH CDR2 comprising a sequence having at most two (e.g., one, two, zero) amino acid modifications relative to SEQ ID NO: 5; and (iii) a VH CDR3 comprising a sequence having at most two (e.g., one, two, zero) amino acid modifications relative to SEQ ID NO: 9.

In some embodiments, the single domain antibody comprises a heavy chain variable (VH) region comprising: (i) a VH CDR1 comprising a sequence having at most two (e.g., one, two, zero) amino acid modifications relative to SEQ ID NO: 3; (ii) a VH CDR2 comprising a sequence having at most two (e.g., one, two, zero) amino acid modifications relative to SEQ ID NO: 6; and (iii) a VH CDR3 comprising a sequence having at most two (e.g., one, two, zero) amino acid modifications relative to SEQ ID NO: 8.

In some embodiments, the single domain antibody comprises a heavy chain variable (VH) region comprising: (i) a VH CDR1 comprising a sequence having at most two (e.g., one, two, zero) amino acid modifications relative to SEQ ID NO: 2; (ii) a VH CDR2 comprising a sequence having at most two (e.g., one, two, zero) amino acid modifications relative to SEQ ID NO: 7; and (iii) a VH CDR3 comprising a sequence having at most two (e.g., one, two, zero) amino acid modifications relative to SEQ ID NO: 10.

In some embodiments, the single domain antibody comprises a heavy chain variable (VH) region comprising: (i) a VH CDR1 comprising a sequence having at most two (e.g., one, two, zero) amino acid modifications relative to SEQ ID NO: 11; (ii) a VH CDR2 comprising a sequence having at most two (e.g., one, two, zero) amino acid modifications relative to SEQ ID NO: 12; and (iii) a VH CDR3 comprising a sequence having at most two (e.g., one, two, zero) amino acid modifications relative to SEQ ID NO: 13.

In some embodiments, each amino acid modification, if any, is a conservative amino acid substitution. In some embodiments, each amino acid modification, if any, is a conservative amino acid substitution listed in Table A1.

In some embodiments, the VH CDR1 comprises a sequence having at most one amino acid modification relative to SEQ ID NO: 1, SEQ ID NO: 2, or SEQ ID NO: 3. In some embodiments, the VH CDR2 comprises a sequence having at most one amino acid modification relative to SEQ ID NO: 4, SEQ ID NO: 5, SEQ ID NO: 6, or SEQ ID NO: 7. In some embodiments, the VH CDR3 comprises a sequence having at most one amino acid modification relative to SEQ ID NO: 8, SEQ ID NO: 9, or SEQ ID NO: 10. In some embodiments, the at most one amino acid modification is an amino acid substitution. In some embodiments, the at most one amino acid modification is a conservative amino acid substitution. In some embodiments, the at most one amino acid modification is an amino acid deletion. In some embodiments, the at most one amino acid modification is an amino acid addition.

In some embodiments, the VH CDR1 comprises a sequence having at most one amino acid modification relative to SEQ ID NO: 11. In some embodiments, the VH CDR2 comprises a sequence having at most one amino acid modification relative to SEQ ID NO: 12. In some embodiments, the VH CDR3 comprises a sequence having at most one amino acid modification relative to SEQ ID NO: 13. In some embodiments, the at most one amino acid modification is an amino acid substitution. In some embodiments, the at most one amino acid modification is a conservative amino acid substitution. In some embodiments, the at most one amino acid modification is an amino acid deletion. In some embodiments, the at most one amino acid modification is an amino acid addition.

In some embodiments, the VH CDR1 comprises a sequence chosen from SEQ ID NO: 1, SEQ ID NO: 2, and SEQ ID NO: 3. In some embodiments, the VH CDR2 comprises a sequence chosen from SEQ ID NO: 4, SEQ ID NO: 5, SEQ ID NO: 6, and SEQ ID NO: 7. In some embodiments, the VH CDR3 comprises a sequence chosen from SEQ ID NO: 8, SEQ ID NO: 9, and SEQ ID NO: 10.

In some embodiments, the single domain antibody comprises a heavy chain variable (VH) region comprising:
(i) a VH complementarity determining region one (CDR1) comprising a sequence chosen from SEQ ID NO: 1, SEQ ID NO: 2, and SEQ ID NO: 3;
(ii) a VH CDR2 comprising a sequence chosen from SEQ ID NO: 4, SEQ ID NO: 5, SEQ ID NO: 6, and SEQ ID NO: 7; and
(iii) a VH CDR3 comprising a sequence chosen from SEQ ID NO: 8, SEQ ID NO: 9, and SEQ ID NO: 10.

In some embodiments, the single domain antibody comprises a heavy chain variable (VH) region comprising:
(a) a VH CDR1, a VH CDR2, and a VH CDR3 comprising the sequences of SEQ ID NOs: 1, 4, and 8, respectively;
(b) a VH CDR1, a VH CDR2, and a VH CDR3 comprising the sequences of SEQ ID NOs: 2, 5, and 9, respectively;
(c) a V CDR1, a VH CDR2, and a VH CDR3 comprising the sequences of SEQ ID NOs: 3, 6, and 8, respectively;
(d) a VH CDR1, a VH CDR2, and a VH CDR3 comprising the sequences of SEQ ID NOs: 2, 7, and 10, respectively; o(e) a VH CDR1, a VH CDR2, and a VH CDR3 comprising the sequences of SEQ ID NOs: 11, 12, and 13, respectively.

In some embodiments, the single domain antibody comprises a heavy chain variable (VH) region comprising a VH CDR1, a VH CDR2, and a VH CDR3 comprising the sequences of SEQ ID NOs: 1, 4, and 8, respectively. In some embodiments, the single domain antibody comprises a heavy chain variable (VH) region comprising a VH CDR1, a VH CDR2, and a VH CDR3 comprising the sequences of SEQ ID NOs: 2, 5, and 9, respectively. In some embodiments, the single domain antibody comprises a heavy chain variable (VH) region comprising a VH CDR1, a VH CDR2, and a VH CDR3 comprising the sequences of SEQ ID NOs: 3, 6, and 8, respectively. In some embodiments, the single domain antibody comprises a heavy chain variable (VH) region comprising a VH CDR1, a VH CDR2, and a VH CDR3 comprising the sequences of SEQ ID NOs: 2, 7, and 10, respectively. In some embodiments, the single domain antibody comprises a heavy chain variable (VH) region comprising a VH CDR1, a VH CDR2, and a VH CDR3 comprising the sequences of SEQ ID NOs: 11, 12, and 13, respectively.

In some embodiments, the single domain antibody comprises a heavy chain variable (VH) region comprising the CDR1, CDR2, and CDR3 of any one of SEQ ID NOs: 14-18. In some embodiments, the single domain antibody comprises a heavy chain variable (VH) region comprising the CDR1, CDR2, and CDR3 of SEQ ID NO: 14. In some embodiments, In some embodiments, the single domain antibody comprises a heavy chain variable (VH) region comprising the CDR1, CDR2, and CDR3 of SEQ ID NO: 15. In some embodiments, the single domain antibody comprises a heavy chain variable (VH) region comprising the CDR1, CDR2, and CDR3 of SEQ ID NO: 16. In some embodiments, the single domain antibody comprises a heavy chain variable (VH) region comprising the CDR1, CDR2, and CDR3 of SEQ ID NO: 17. In some embodiments, the single domain antibody comprises a heavy chain variable (VH) region comprising the CDR1, CDR2, and CDR3 of SEQ ID NO: 18.

In some embodiments, the VH CDR1, VH CDR2, and VH CDR3 sequences are present in a human VH framework.

In some embodiments, the single domain antibody comprises a heavy chain variable (VH) region having at least 80% (such as, e.g., 80%, 85%, 90%, 95%, 98%, 99%, at least 85%, at least 90%, at least 95%, at least 98%, at least 99%) sequence identity to any one of SEQ ID NOs: 14-18. In some embodiments, the single domain antibody comprises a heavy chain variable (VH) region having at least 85% (such as, e.g., 85%, 90%, 95%, 98%, 99%, at least 90%, at least 95%, at least 98%, at least 99%) sequence identity to any one of SEQ ID NOs: 14-18. In some embodiments, the single domain antibody comprises a heavy chain variable (VH) region having at least 90% (such as, e.g., 90%, 95%, 98%, 99%, at least 95%, at least 98%, at least 99%) sequence identity to any one of SEQ ID NOs: 14-18. In some embodiments, the single domain antibody comprises a heavy chain variable (VH) region having at least 95% sequence identity to any one of SEQ ID NOs: 14-18. In some embodiments, the single domain antibody comprises a heavy chain variable (VH) region having at least 99% sequence identity to any one of SEQ ID NOs: 14-18.

In some embodiments, the single domain antibody comprises a heavy chain variable (VH) region having at least 80% (such as, e.g., 80%, 85%, 90%, 95%, 98%, 99%, at least 85%, at least 90%, at least 95%, at least 98%, at least 99%) sequence identity to SEQ ID NO: 14. In some embodiments, the single domain antibody comprises a heavy chain variable (VH) region having at least 80% (such as, e.g., 80%, 85%, 90%, 95%, 98%, 99%, at least 85%, at least 90%, at least 95%, at least 98%, at least 99%) sequence identity to SEQ ID NO: 15. In some embodiments, the single domain antibody comprises a heavy chain variable (VH) region having at least 80% (such as, e.g., 80%, 85%, 90%, 95%, 98%, 99%, at least 85%, at least 90%, at least 95%, at least 98%, at least 99%) sequence identity to SEQ ID NO: 16. In some embodiments, the single domain antibody comprises a heavy chain variable (VH) region having at least 80% (such as, e.g., 80%, 85%, 90%, 95%, 98%, 99%, at least 85%, at least 90%, at least 95%, at least 98%, at least 99%) sequence identity to SEQ ID NO: 17. In some embodiments, the single domain antibody comprises a heavy chain variable (VH) region having at least 80% (such as, e.g., 80%, 85%, 90%, 95%, 98%, 99%, at least 85%, at least 90%, at least 95%, at least 98%, at least 99%) sequence identity to SEQ ID NO: 18.

In some embodiments, the single domain antibody comprises a heavy chain variable (VH) region chosen from SEQ ID NOs: 14-18.

In some embodiments, the single domain antibody comprises the heavy chain variable (VH) region of SEQ ID NO: 14. In some embodiments, the single domain antibody comprises the heavy chain variable (VH) region of SEQ ID NO: 15. In some embodiments, the single domain antibody comprises the heavy chain variable (VH) region of SEQ ID NO: 16. In some embodiments, the single domain antibody comprises the heavy chain variable (VH) region of SEQ ID NO: 17. In some embodiments, the single domain antibody comprises the heavy chain variable (VH) region of SEQ ID NO: 18.

In some embodiments, the c-Kit binding protein specifically binds to human c-Kit. In some embodiments, the c-Kit binding protein binds to human c-Kit with a K_{D} of from about 10⁻⁹ M to about 10⁻⁶ M. In some embodiments, the c-Kit binding protein binds to human c-Kit with a K_{D} of ≤5×10⁻⁷ M. In some embodiments, the c-Kit binding protein binds to human c-Kit with a K_{D} of ≤1×10⁻⁷ M. In some embodiments, the c-Kit binding protein binds to human c-Kit with a K_{D} of ≤5×10⁻⁸ M. In some embodiments, the c-Kit binding protein binds to human c-Kit with a K_{D} of ≤2×10⁻⁸ M. In some embodiments, the c-Kit binding protein binds to human c-Kit with a K_{D} of ≤1×10⁻⁸ M. In some embodiments, the c-Kit binding protein binds to human c-Kit with a K_{D} of ≤1×10⁻⁹ M.

In some embodiments, the c-Kit binding protein specifically binds to a GNNK− isoform of human c-Kit. In some embodiments, the c-Kit binding protein binds to a GNNK− isoform of human c-Kit with a K_{D} of from about 10⁻⁹ M to about 10⁻⁶ M. In some embodiments, the c-Kit binding protein binds to a GNNK− isoform of human c-Kit with a K_{D} of ≤5×10⁻⁷ M. In some embodiments, the c-Kit binding protein binds to a GNNK− isoform of human c-Kit with a K_{D} of ≤1×10⁻⁷ M. In some embodiments, the c-Kit binding protein binds to a GNNK− isoform of human c-Kit with a K_{D} of ≤5×10⁻⁸ M. In some embodiments, the c-Kit binding protein binds to a GNNK− isoform of human c-Kit with a K_{D} of ≤2×10⁻⁸ M. In some embodiments, the c-Kit binding protein binds to a GNNK− isoform of human c-Kit with a K_{D} of ≤1×10⁻⁸ M. In some embodiments, the c-Kit binding protein binds to a GNNK− isoform of human c-Kit with a K_{D} of ≤1×10⁻⁹ M.

In some embodiments, the c-Kit binding protein specifically binds to a GNNK+ isoform of human c-Kit. In some embodiments, the c-Kit binding protein binds to a GNNK+ isoform of human c-Kit with a K_{D} of from about 10⁻⁹ M to about 10⁻⁶ M. In some embodiments, the c-Kit binding protein binds to a GNNK+ isoform of human c-Kit with a K_{D} of ≤5×10⁻⁷ M. In some embodiments, the c-Kit binding protein binds to a GNNK+ isoform of human c-Kit with a K_{D} of ≤1×10⁻⁷ M. In some embodiments, the c-Kit binding protein binds to a GNNK+ isoform of human c-Kit with a K_{D} of ≤5×10⁻⁸ M. In some embodiments, the c-Kit binding protein binds to a GNNK+ isoform of human c-Kit with a K_{D} of ≤2×10⁻⁸ M. In some embodiments, the c-Kit binding protein binds to a GNNK+ isoform of human c-Kit with a K_{D} of ≤1×10⁻⁸ M. In some embodiments, the c-Kit binding protein binds to a GNNK+ isoform of human c-Kit with a K_{D} of ≤1×10⁻⁹ M.

In some embodiments, the c-Kit binding protein further binds to one or more target antigens other than c-Kit. In some embodiments, the c-Kit binding protein is multispecific. In some embodiments, the c-Kit binding protein is bispecific.

In some embodiments, the c-Kit binding protein further specifically binds to CD3. In some embodiments, the c-Kit binding protein further specifically binds to human CD3. In some embodiments, the c-Kit binding protein binds to human CD3 with a K_{D} of from about 10⁻⁹ M to about 10⁻⁶ M. In some embodiments, the c-Kit binding protein binds to human CD3 with a K_{D} of ≤5×10⁻⁷ M. In some embodiments, the c-Kit binding protein binds to human CD3 with a K_{D} of ≤1×10⁻⁷ M. In some embodiments, the c-Kit binding protein binds to human CD3 with a K_{D} of ≤5×10⁻⁸ M. In some embodiments, the c-Kit binding protein binds to human CD3 with a K_{D} of ≤2×10⁻⁸ M. In some embodiments, the c-Kit binding protein binds to human CD3 with a K_{D} of ≤1×10⁻⁸ M. In some embodiments, the c-Kit binding protein binds to human CD3 with a K_{D} of ≤1×10⁻⁹ M.

In some embodiments, the c-Kit binding protein binds to human c-Kit and/or CD3 with a K_{D} of from about 10⁻⁹ M to about 10⁻⁶ M. In some embodiments, the c-Kit binding protein binds to human c-Kit and/or CD3 with a K_{D} of ≤5×10⁻⁷ M. In some embodiments, the c-Kit binding protein binds to human c-Kit and/or CD3 with a K_{D} of ≤1×10⁻⁷ M. In some embodiments, the c-Kit binding protein binds to human c-Kit and/or CD3 with a K_{D} of ≤5×10⁻⁸ M. In some embodiments, the c-Kit binding protein binds to human c-Kit and/or CD3 with a K_{D} of ≤2×10⁻⁸ M. In some embodiments, the c-Kit binding protein binds to human c-Kit and/or CD3 with a K_{D} of ≤1×10⁻⁸ M. In some embodiments, the c-Kit binding protein binds to human c-Kit and/or CD3 with a K_{D} of ≤1×10⁻⁹ M.

In some embodiments, the c-Kit binding protein further specifically binds to human CD3 epsilon. In some embodiments, the c-Kit binding protein binds to an epitope on CD3 comprising at least one residue selected from CD3 epsilon (SEQ ID NO: 69): K73 and S83; and CD3 delta (SEQ ID NO: 70) K82 and C93. In some embodiments, the epitope on CD3 comprises the region of CD3 delta defined by K82, E83, S84, T85, V86, Q87, V88, H89, Y90, R91, M92, C93. In some embodiments, the epitope on CD3 comprises the region of CD3 epsilon defined by K73, N74, 175, G76, S77, D78, E79, D80, H81, L82, S83. In some embodiments, the epitope comprises a conformational epitope with residues of both CD3 delta and CD3 epsilon. In some embodiments, the conformational epitope comprises each of residues CD3ε K73 and S83; CD3δ K82 and C93.

In some embodiments, the c-Kit binding protein further comprises a CD3-binding VH region. In some embodiments, the c-Kit binding protein further comprises a CD3-binding VH region that is paired with a light chain (VL) region.

In some embodiments, the CD3-binding VH region may belong to a family of closely related single domain antibodies that specifically bind to human CD3. The single domain antibodies of this family comprise a set of CDR sequences as defined herein and as shown in Tables S5 and S6, and are exemplified by the provided heavy chain variable region (VH) sequences of SEQ ID NOs: 31-48 as set forth in Table S7. Multi-specific molecules comprising these CD3-binding VH domains and their associated light chain variable domain (as described in Tables S8 and S9) have advantageous properties, for example, as described in published PCT application publication number WO2018/052503, the disclosure of which is incorporated by reference herein in its entirety. Any of the single domain antibodies described herein that specifically binds to c-Kit can be combined with the CD3-binding domains and fixed light chain domains described herein to generate a multispecific c-Kit binding protein.

TABLE S5
Anti-CD3 Heavy Chain Antibody CDR1, CDR2 and CDR3 Amino Acid
Sequences

| Clone | | | |
|---|---|---|---|
| ID # | SEQ aa CDR1 | SEQ_aa_CDR2 | SEQ_aa CDR3 |
| 312557 | GFTFDDYA | ISWNSGSI | AKDSRGYGDYRLGGAY |
| | (SEQ ID NO: 20) | (SEQ ID NO: 26) | (SEQ ID NO: 27) |
| 308261 | GFTFDDYA | ISWNSGSI | AKDSRGYGDYRLGGAY |
| | (SEQ ID NO: 20) | (SEQ ID NO: 26) | (SEQ ID NO: 27) |
| 308159 | GFTFDDYA | ISWNSGSI | AKDSRGYGDYSRGGAY |
| | (SEQ ID NO: 20) | (SEQ ID NO: 26) | (SEQ ID NO: 28) |
| 308160 | GFTFDDYA | ISWNSGSI | AKDSRGYGDYSLGGAY |
| | (SEQ ID NO: 20) | (SEQ ID NO: 26) | (SEQ ID NO: 29) |
| 308256 | GFTFDDYA | ISWNSGSI | AKDSRGYGDYSLGGAY |
| | (SEQ ID NO: 20) | (SEQ ID NO: 26) | (SEQ ID NO: 29) |
| 312585 | GFTFANYA | ISWNSGSI | AKDSRGYGDYSRGGAY |
| | (SEQ ID NO: 21) | (SEQ ID NO: 26) | (SEQ ID NO: 28) |
| 312614 | GFTFNNYA | ISWNSGSI | AKDSRGYGDYSRGGAY |
| | (SEQ ID NO: 22) | (SEQ ID NO: 26) | (SEQ ID NO: 28) |
| 312583 | GFTFADYA | ISWNSGSI | AKDSRGYGDYSRGGAY |
| | (SEQ ID NO: 23) | (SEQ ID NO: 26) | (SEQ ID NO: 28) |
| 312586 | GFTFDNYA | ISWNSGSI | AKDSRGYGDYSRGGAY |
| | (SEQ ID NO: 24) | (SEQ ID NO: 26) | (SEQ ID NO: 28) |
| 312624 | GFTFDNYA | ISWNSGSI | AKDSRGYGDYSRGGAY |
| | (SEQ ID NO: 24) | (SEQ ID NO: 26) | (SEQ ID NO: 28) |
| 312578 | GFTFDDYA | ISWNSGSI | AKDSRGYGDYSRGGAY |
| | (SEQ ID NO: 20) | (SEQ ID NO: 26) | (SEQ ID NO: 28) |
| 312620 | GFTFDDYA | ISWNSGSI | AKDSRGYGSYSRGGAY |
| | (SEQ ID NO: 20) | (SEQ ID NO: 26) | (SEQ ID NO: 30) |
| 312634 | GFTFHNYA | ISWNSGSI | AKDSRGYGDYSLGGAY |
| | (SEQ ID NO: 25) | (SEQ ID NO: 26) | (SEQ ID NO: 29) |
| 312579 | GFTFDDYA | ISWNSGSI | AKDSRGYGDYSRGGAY |
| | (SEQ ID NO: 20) | (SEQ ID NO: 26) | (SEQ ID NO: 28) |
| 312630 | GFTFDNYA | ISWNSGSI | AKDSRGYGDYSLGGAY |
| | (SEQ ID NO: 24) | (SEQ ID NO: 26) | (SEQ ID NO: 29) |
| 312570 | GFTFDDYA | ISWNSGSI | AKDSRGYGDYRLGGAY |
| | (SEQ ID NO: 20) | (SEQ ID NO: 26) | (SEQ ID NO: 27) |
| 312567 | GFTFDDYA | ISWNSGSI | AKDSRGYGDYRLGGAY |
| | (SEQ ID NO: 20) | (SEQ ID NO: 26) | (SEQ ID NO: 27) |
| 312558 | GFTFDDYA | ISWNSGSI | AKDSRGYGDYRLGGAY |
| | (SEQ ID NO: 20) | (SEQ ID NO: 26) | (SEQ ID NO: 27) |

TABLE S6
Anti-CD3 Heavy Chain Antibody Unique CDR Amino Acid Sequences

| SEQ_aa_CDR1 | SEQ_aa_CDR2 | SEQ_aa_CDR3 |
|---|---|---|
| GFTFDDYA | ISWNSGSI | AKDSRGYGDYRLGGAY |
| (SEQ ID NO: 20) | (SEQ ID NO: 26) | (SEQ ID NO: 27) |
| GFTFANYA | | AKDSRGYGDYSRGGAY |
| (SEQ ID NO: 21) | | (SEQ ID NO: 28) |
| GFTFNNYA | | AKDSRGYGDYSLGGAY |
| (SEQ ID NO: 22) | | (SEQ ID NO: 29) |
| GFTFADYA | | AKDSRGYGSYSRGGAY |
| (SEQ ID NO: 23) | | (SEQ ID NO: 30) |
| GFTFDNYA | | |
| (SEQ ID NO: 24) | | |
| GFTFHNYA | | |
| (SEQ ID NO: 25) | | |

TABLE S7
Anti-CD3 Heavy Chain Antibody Variable Domain Amino Acid Sequences

| Clone | | SEQ ID |
|---|---|---|
| ID # | SEQ_aa_FR1_FR4 | NO. |
| 312557 | EVQLVESGGGLVQPGRSLRLSCAASGFTFDDYAMHWVRQAPGK | 31 |
| | GLEWVSGISWNSGSIGYADSVKGRFTISRDNAKNSLYLQMNSLR | |
| | AEDTALYYCAKDSRGYGDYRLGGAYWGQGTLVTVSS | |
| 308261 | EVQLVESGGGLVKPGGSLRLSCAASGFTFDDYAMHWVRQAPG | 32 |
| | KGLEWVSGISWNSGSIGYADSVKGRFTISRDNAKNSLYLQMNSL | |
| | RAEDTALYYCAKDSRGYGDYRLGGAYWGQGTLVTVSS | |
| 308159 | EVQLVESGGGLVQPGRSLRLSCAASGFTFDDYAMHWVRQAPGK | 33 |
| | GLEWVSGISWNSGSIGYADSVKGRFTISRDNAKKSLYLQMNSLR | |
| | AEDTALYYCAKDSRGYGDYSRGGAYWGQGTLVTVSS | |
| 308160 | EVQLVESGGGLVQPGRSLRLSCAASGFTFDDYAMHWVRQAPGK | 34 |
| | GLEWVSGISWNSGSIGYADSVKGRFTISRDNAKNSLYLQMNSLR | |
| | PEDTALYYCAKDSRGYGDYSLGGAYWGQGTLVTVSS | |
| 308256 | EVQLVESGGGLVQPGRSLRLSCAASGFTFDDYAMHWVRQAPGK | 35 |
| | GLEWVSGISWNSGSIGYADSVKGRFTISRDNAKNSLYLQMNSLR | |
| | AEDTALYYCAKDSRGYGDYSLGGAYWGQGTLVTVSS | |
| 312585 | EVQLVESGGGLVQPGRSLRLSCAASGFTFANYAMHWVRQAPGK | 36 |
| | GLEWVSGISWNSGSIDYADSVKGRFTISRDNAKNSLYLQMNSLR | |
| | AEDTALYYCAKDSRGYGDYSRGGAYWGQGTLVTVSS | |
| 312614 | EVQLVESGGGLVQPGRSLRLSCAASGFTFNNYAMHWVRQAPGK | 37 |
| | GLEWVSGISWNSGSIGYADSVKGRFTISRDNAKNSLYLQMNSLR | |
| | GEDTALYYCAKDSRGYGDYSRGGAYWGQGTLVTVSS | |
| 312583 | EVQLVESGGGLVQPGGSLRLSCAASGFTFADYAMHWVRQAPG | 38 |
| | KGLEWVSGISWNSGSIGYADSVKGRFTISRDNAKNSLYLQMNSL | |
| | RAEDTALYYCAKDSRGYGDYSRGGAYWGQGTLVTVSS | |
| 312586 | EVQLVESGGGLVQPGRSLRLSCAASGFTFDNYAMHWVRQAPGK | 39 |
| | GLEWVSGISWNSGSIGYADSVKGRFTISRDNAKKSLYLQMNSLR | |
| | AEDTALYYCAKDSRGYGDYSRGGAYWGQGTLVTVSS | |
| 312624 | EVQLVESGGGLVQPGRSLRLSCAASGFTFDNYAMHWVRQAPGK | 40 |
| | GLEWVSGISWNSGSIGYADSVKGRFTISRDNAKNSLYLQMNSLR | |
| | AEDTALYYCAKDSRGYGDYSRGGAYWGQGTLVTVSS | |
| 312578 | EVQLVESGGGLVQPGRSLRLSCAASGFTFDDYAMHWVRQAPGK | 41 |
| | GLEWVSGISWNSGSIGYADSVKGRFTISRDNAKNSLYLQMNSLR | |
| | AEDTALYYCAKDSRGYGDYSRGGAYWGQGTLVTVSS | |
| 312620 | EVQLVESGGGLVQPGRSLRLSCAASGFTFDDYAMHWVRQAPGK | 42 |
| | GLEWVSGISWNSGSIGYADSVKGRFTISRDNAKNSLYLQMNSLR | |
| | PEDTALYYCAKDSRGYGSYSRGGAYWGQGTLVTVSS | |
| 312634 | EVQLVESGGGLVQPGRSLRLSCAASGFTFHNYAMHWVRQAPGK | 43 |
| | GLEWVSGISWNSGSIGYADSVKGRFTISRDNAKNSLYLQMNSLR | |
| | AEDTALYYCAKDSRGYGDYSLGGAYWGQGTLVTVSS | |
| 312579 | EVQLVESGGGLVQPGGSLRLSCAASGFTFDDYAMHWVRQAPG | 44 |
| | KGLEWVSGISWNSGSIGYADSVKGRFTISRDNAKNSLYLQMNSL | |
| | RAEDTALYYCAKDSRGYGDYSRGGAYCGQGTLVTVSS | |
| 312630 | EVQLVESGGGLVQPGRSLRLSCAASGFTFDNYAMHWVRQAPGK | 45 |
| | GLEWVSGISWNSGSIGYADSVKGRFTISRDNAKKSLYLQMNSLR | |
| | AEDTALYYCAKDSRGYGDYSLGGAYWGQGTLVTVSS | |
| 312570 | EVQLVESGGGLVQPGRSLRLSCAASGFTFDDYAMHWVRQAPGK | 46 |
| | GLEWVSGISWNSGSIGYADSVKGRFTISRDNAKKSLYLQMNSLR | |
| | AEDTALYYCAKDSRGYGDYRLGGAYWGQGTLVTVSS | |
| 312567 | EVQLVESGGGLVQPGRSLRLSCEASGFTFDDYAMHWVRQAPGK | 47 |
| | GLEWVSGISWNSGSIGYADSVKGRFTISRDNAKNSLYLQMNSLR | |
| | AEDTALYYCAKDSRGYGDYRLGGAYWGQGTLVTVSS | |
| 312558 | EVQLVESGGGLVQPGRSLRLSCAASGFTFDDYAMHWVRQAPGK | 48 |
| | GLEWVSGISWNSGSIGYADSVKGRFTISRDNAKNSLYLQMNSLR | |
| | PEDTALYYCAKDSRGYGDYRLGGAYWGQGTLVTVSS | |

TABLE S8
Anti-CD3 Arm Fixed Light Chain Antibody CDR1, CDR2 and
CDR3 Amino Acid Sequences

| SEQ_aa_CDR1 | SEQ_aa_CDR2 | SEQ_aa_CDR3 |
|---|---|---|
| QSVSSN | GAS | QQYNNWPWT |
| (SEQ ID NO: 49) | (SEQ ID NO: 50) | (SEQ ID NO: 51) |

TABLE S9
Anti-CD3 Arm Fixed Light Chain Antibody Variable Domain Amino Acid
Sequence

| Clone | | SEQ ID |
|---|---|---|
| ID # | SEQ_aa_FR1_FR4 | NO. |
| 312325 | EIVMTQSPATLSVSPGERATLSCRASQSVSSNLAWYQQKPG | 52 |
| | QAPRLLIYGASTRATGIPARFSGSGSGTEFTLTISSLQSEDFA | |
| | VYYCQQYNNWPWTFGQGTKVEIK | |

In some embodiments, the CD3-binding VH region comprises:
(i) a VH complementarity determining region one (CDR1) comprising a sequence having at most two (such as, e.g., zero, one, or two) amino acid modifications relative to any one of SEQ ID NOs: 20-25;
(ii) a VH CDR2 comprising a sequence having at most two (such as, e.g., zero, one, or two) amino acid modifications relative to SEQ ID NO: 26; and
(iii) a VH CDR3 comprising a sequence having at most two (such as, e.g., zero, one, or two) amino acid modifications relative to any one of SEQ ID NOs: 27-30.

In some embodiments, the CD3-binding VH region comprises: (i) a VH complementarity determining region one (CDR1) comprising a sequence having at most two (such as, e.g., zero, one, or two) amino acid modifications relative to SEQ ID NO: 20; (ii) a VH CDR2 comprising a sequence having at most two (such as, e.g., zero, one, or two) amino acid modifications relative to SEQ ID NO: 26; and (iii) a VH CDR3 comprising a sequence having at most two (such as, e.g., zero, one, or two) amino acid modifications relative to SEQ ID NO: 27. In some embodiments, the CD3-binding VH CDR2 comprises the sequence of SEQ ID NO: 26.

In some embodiments, the CD3-binding VH region comprises: (i) a VH complementarity determining region one (CDR1) comprising a sequence having at most two (such as, e.g., zero, one, or two) amino acid modifications relative to SEQ ID NO: 20; (ii) a VH CDR2 comprising a sequence having at most two (such as, e.g., zero, one, or two) amino acid modifications relative to SEQ ID NO: 26; and (iii) a VH CDR3 comprising a sequence having at most two (such as, e.g., zero, one, or two) amino acid modifications relative to SEQ ID NO: 28. In some embodiments, the CD3-binding VH CDR2 comprises the sequence of SEQ ID NO: 26.

In some embodiments, the CD3-binding VH region comprises: (i) a VH complementarity determining region one (CDR1) comprising a sequence having at most two (such as, e.g., zero, one, or two) amino acid modifications relative to SEQ ID NO: 20; (ii) a VH CDR2 comprising a sequence having at most two (such as, e.g., zero, one, or two) amino acid modifications relative to SEQ ID NO: 26; and (iii) a VH CDR3 comprising a sequence having at most two (such as, e.g., zero, one, or two) amino acid modifications relative to SEQ ID NO: 29. In some embodiments, the CD3-binding VH CDR2 comprises the sequence of SEQ ID NO: 26.

In some embodiments, the CD3-binding VH region comprises: (i) a VH complementarity determining region one (CDR1) comprising a sequence having at most two (such as, e.g., zero, one, or two) amino acid modifications relative to SEQ ID NO: 21; (ii) a VH CDR2 comprising a sequence having at most two (such as, e.g., zero, one, or two) amino acid modifications relative to SEQ ID NO: 26; and (iii) a VH CDR3 comprising a sequence having at most two (such as, e.g., zero, one, or two) amino acid modifications relative to SEQ ID NO: 28. In some embodiments, the CD3-binding VH CDR2 comprises the sequence of SEQ ID NO: 26.

In some embodiments, the CD3-binding VH region comprises: (i) a VH complementarity determining region one (CDR1) comprising a sequence having at most two (such as, e.g., zero, one, or two) amino acid modifications relative to SEQ ID NO: 22; (ii) a VH CDR2 comprising a sequence having at most two (such as, e.g., zero, one, or two) amino acid modifications relative to SEQ ID NO: 26; and (iii) a VH CDR3 comprising a sequence having at most two (such as, e.g., zero, one, or two) amino acid modifications relative to SEQ ID NO: 28. In some embodiments, the CD3-binding VH CDR2 comprises the sequence of SEQ ID NO: 26.

In some embodiments, the CD3-binding VH region comprises: (i) a VH complementarity determining region one (CDR1) comprising a sequence having at most two (such as, e.g., zero, one, or two) amino acid modifications relative to SEQ ID NO: 23; (ii) a VH CDR2 comprising a sequence having at most two (such as, e.g., zero, one, or two) amino acid modifications relative to SEQ ID NO: 26; and (iii) a VH CDR3 comprising a sequence having at most two (such as, e.g., zero, one, or two) amino acid modifications relative to SEQ ID NO: 28. In some embodiments, the CD3-binding VH CDR2 comprises the sequence of SEQ ID NO: 26.

In some embodiments, the CD3-binding VH region comprises: (i) a VH complementarity determining region one (CDR1) comprising a sequence having at most two (such as, e.g., zero, one, or two) amino acid modifications relative to SEQ ID NO: 24; (ii) a VH CDR2 comprising a sequence having at most two (such as, e.g., zero, one, or two) amino acid modifications relative to SEQ ID NO: 26; and (iii) a VH CDR3 comprising a sequence having at most two (such as, e.g., zero, one, or two) amino acid modifications relative to SEQ ID NO: 28. In some embodiments, the CD3-binding VH CDR2 comprises the sequence of SEQ ID NO: 26.

In some embodiments, the CD3-binding VH region comprises: (i) a VH complementarity determining region one (CDR1) comprising a sequence having at most two (such as, e.g., zero, one, or two) amino acid modifications relative to SEQ ID NO: 20; (ii) a VH CDR2 comprising a sequence having at most two (such as, e.g., zero, one, or two) amino acid modifications relative to SEQ ID NO: 26; and (iii) a VH CDR3 comprising a sequence having at most two (such as, e.g., zero, one, or two) amino acid modifications relative to SEQ ID NO: 30. In some embodiments, the CD3-binding VH CDR2 comprises the sequence of SEQ ID NO: 26.

In some embodiments, the CD3-binding VH region comprises: (i) a VH complementarity determining region one (CDR1) comprising a sequence having at most two (such as, e.g., zero, one, or two) amino acid modifications relative to SEQ ID NO: 25; (ii) a VH CDR2 comprising a sequence having at most two (such as, e.g., zero, one, or two) amino acid modifications relative to SEQ ID NO: 26; and (iii) a VH CDR3 comprising a sequence having at most two (such as, e.g., zero, one, or two) amino acid modifications relative to SEQ ID NO: 29. In some embodiments, the CD3-binding VH CDR2 comprises the sequence of SEQ ID NO: 26.

In some embodiments, the CD3-binding VH region comprises: (i) a VH complementarity determining region one (CDR1) comprising a sequence having at most two (such as, e.g., zero, one, or two) amino acid modifications relative to SEQ ID NO: 24; (ii) a VH CDR2 comprising a sequence having at most two (such as, e.g., zero, one, or two) amino acid modifications relative to SEQ ID NO: 26; and (iii) a VH CDR3 comprising a sequence having at most two (such as, e.g., zero, one, or two) amino acid modifications relative to SEQ ID NO: 29. In some embodiments, the CD3-binding VH CDR2 comprises the sequence of SEQ ID NO: 26.

In some embodiments, each amino acid modification, if any, is a conservative amino acid substitution. In some embodiments, each amino acid modification, if any, is a conservative amino acid substitution listed in Table A1.

In some embodiments, the CD3-binding VH CDR1 comprises a sequence having at most one amino acid modification relative to any one of SEQ ID NO: 20-25. In some embodiments, the CD3-binding VH CDR2 comprises a sequence having at most one amino acid modification relative to SEQ ID NO: 26. In some embodiments, the CD3-binding VH CDR3 comprises a sequence having at most one amino acid modification relative to any one of SEQ ID NOs: 27-30. In some embodiments, the at most one amino acid modification is an amino acid substitution. In some embodiments, the at most one amino acid modification is a conservative amino acid substitution. In some embodiments, the at most one amino acid modification is an amino acid deletion. In some embodiments, the at most one amino acid modification is an amino acid addition.

In some embodiments, the CD3-binding VH CDR1 comprises a sequence chosen from SEQ ID NOs: 20-25. In some embodiments, the CD3-binding VH CDR1 comprises the sequence of SEQ ID NO: 20. In some embodiments, the CD3-binding VH CDR1 comprises the sequence of SEQ ID NO: 21. In some embodiments, the CD3-binding VH CDR1 comprises the sequence of SEQ ID NO: 22. In some embodiments, the CD3-binding VH CDR1 comprises the sequence of SEQ ID NO: 23. In some embodiments, the CD3-binding VH CDR1 comprises the sequence of SEQ ID NO: 24. In some embodiments, the CD3-binding VH CDR1 comprises the sequence of SEQ ID NO: 25.

In some embodiments, the CD3-binding VH CDR2 comprises the sequence of SEQ ID NO: 26.

In some embodiments, the CD3-binding VH CDR3 comprises a sequence chosen from SEQ ID NOs: 27-30. In some embodiments, the CD3-binding VH CDR3 comprises the sequence of SEQ ID NO: 27. In some embodiments, the CD3-binding VH CDR3 comprises the sequence of SEQ ID NO: 28. In some embodiments, the CD3-binding VH CDR3 comprises the sequence of SEQ ID NO: 29. In some embodiments, the CD3-binding VH CDR3 comprises the sequence of SEQ ID NO: 30.

In some embodiments, the full set of VH CDRs 1, 2, and 3 (combined) in the CD3-binding VH region has at least 80% (such as, e.g., 80%, 85%, 90%, 95%, 98%, 99%, at least 85%, at least 90%, at least 95%, at least 98%, at least 99%) sequence identity to the VH CDRs 1, 2, and 3 of any one of SEQ ID NOs: 31-48. In some embodiments, the full set of VH CDRs 1, 2, and 3 (combined) in the CD3-binding VH region has at least 85% (such as, e.g., 85%, 90%, 95%, 98%, 99%, at least 90%, at least 95%, at least 98%, at least 99%) sequence identity to the VH CDRs 1, 2, and 3 of any one of SEQ ID NOs: 31-48. In some embodiments, the full set of VH CDRs 1, 2, and 3 (combined) in the CD3-binding VH region has at least 90% (such as, e.g., 90%, 95%, 98%, 99%, at least 95%, at least 98%, at least 99%) sequence identity to the VH CDRs 1, 2, and 3 of any one of SEQ ID NOs: 31-48. In some embodiments, the full set of VH CDRs 1, 2, and 3 (combined) in the CD3-binding VH region has at least 95% sequence identity to the VH CDRs 1, 2, and 3 of any one of SEQ ID NOs: 31-48. In some embodiments, the full set of VH CDRs 1, 2, and 3 (combined) in the CD3-binding VH region has at least 99% sequence identity to the VH CDRs 1, 2, and 3 of any one of SEQ ID NOs: 31-48.

In some embodiments, the full set of VH CDRs 1, 2, and 3 (combined) in the CD3-binding VH region has at least 80% (such as, e.g., 80%, 85%, 90%, 95%, 98%, 99%, at least 85%, at least 90%, at least 95%, at least 98%, at least 99%) sequence identity to the VH CDRs 1, 2, and 3 of SEQ ID NO: 31. In some embodiments, the full set of VH CDRs 1, 2, and 3 (combined) in the CD3-binding VH region has at least 80% (such as, e.g., 80%, 85%, 90%, 95%, 98%, 99%, at least 85%, at least 90%, at least 95%, at least 98%, at least 99%) sequence identity to the VH CDRs 1, 2, and 3 of SEQ ID NO: 32. In some embodiments, the full set of VH CDRs 1, 2, and 3 (combined) in the CD3-binding VH region has at least 80% (such as, e.g., 80%, 85%, 90%, 95%, 98%, 99%, at least 85%, at least 90%, at least 95%, at least 98%, at least 99%) sequence identity to the VH CDRs 1, 2, and 3 of SEQ ID NO: 33. In some embodiments, the full set of VH CDRs 1, 2, and 3 (combined) in the CD3-binding VH region has at least 80% (such as, e.g., 80%, 85%, 90%, 95%, 98%, 99%, at least 85%, at least 90%, at least 95%, at least 98%, at least 99%) sequence identity to the VH CDRs 1, 2, and 3 of SEQ ID NO: 34. In some embodiments, the full set of VH CDRs 1, 2, and 3 (combined) in the CD3-binding VH region has at least 80% (such as, e.g., 80%, 85%, 90%, 95%, 98%, 99%, at least 85%, at least 90%, at least 95%, at least 98%, at least 99%) sequence identity to the VH CDRs 1, 2, and 3 of SEQ ID NO: 35. In some embodiments, the full set of VH CDRs 1, 2, and 3 (combined) in the CD3-binding VH region has at least 80% (such as, e.g., 80%, 85%, 90%, 95%, 98%, 99%, at least 85%, at least 90%, at least 95%, at least 98%, at least 99%) sequence identity to the VH CDRs 1, 2, and 3 of SEQ ID NO: 36. In some embodiments, the full set of VH CDRs 1, 2, and 3 (combined) in the CD3-binding VH region has at least 80% (such as, e.g., 80%, 85%, 90%, 95%, 98%, 99%, at least 85%, at least 90%, at least 95%, at least 98%, at least 99%) sequence identity to the VH CDRs 1, 2, and 3 of SEQ ID NO: 37. In some embodiments, the full set of VH CDRs 1, 2, and 3 (combined) in the CD3-binding VH region has at least 80% (such as, e.g., 80%, 85%, 90%, 95%, 98%, 99%, at least 85%, at least 90%, at least 95%, at least 98%, at least 99%) sequence identity to the VH CDRs 1, 2, and 3 of SEQ ID NO: 38. In some embodiments, the full set of VH CDRs 1, 2, and 3 (combined) in the CD3-binding VH region has at least 80% (such as, e.g., 80%, 85%, 90%, 95%, 98%, 99%, at least 85%, at least 90%, at least 95%, at least 98%, at least 99%) sequence identity to the VH CDRs 1, 2, and 3 of SEQ ID NO: 39. In some embodiments, the full set of VH CDRs 1, 2, and 3 (combined) in the CD3-binding VH region has at least 80% (such as, e.g., 80%, 85%, 90%, 95%, 98%, 99%, at least 85%, at least 90%, at least 95%, at least 98%, at least 99%) sequence identity to the VH CDRs 1, 2, and 3 of SEQ ID NO: 40. In some embodiments, the full set of VH CDRs 1, 2, and 3 (combined) in the CD3-binding VH region has at least 80% (such as, e.g., 80%, 85%, 90%, 95%, 98%, 99%, at least 85%, at least 90%, at least 95%, at least 98%, at least 99%) sequence identity to the VH CDRs 1, 2, and 3 of SEQ ID NO: 41. In some embodiments, the full set of VH CDRs 1, 2, and 3 (combined) in the CD3-binding VH region has at least 80% (such as, e.g., 80%, 85%, 90%, 95%, 98%, 99%, at least 85%, at least 90%, at least 95%, at least 98%, at least 99%) sequence identity to the VH CDRs 1, 2, and 3 of SEQ ID NO: 42. In some embodiments, the full set of VH CDRs 1, 2, and 3 (combined) in the CD3-binding VH region has at least 80% (such as, e.g., 80%, 85%, 90%, 95%, 98%, 99%, at least 85%, at least 90%, at least 95%, at least 98%, at least 99%) sequence identity to the VH CDRs 1, 2, and 3 of SEQ ID NO: 43. In some embodiments, the full set of VH CDRs 1, 2, and 3 (combined) in the CD3-binding VH region has at least 80% (such as, e.g., 80%, 85%, 90%, 95%, 98%, 99%, at least 85%, at least 90%, at least 95%, at least 98%, at least 99%) sequence identity to the VH CDRs 1, 2, and 3 of SEQ ID NO: 44. In some embodiments, the full set of VH CDRs 1, 2, and 3 (combined) in the CD3-binding VH region has at least 80% (such as, e.g., 80%, 85%, 90%, 95%, 98%, 99%, at least 85%, at least 90%, at least 95%, at least 98%, at least 99%) sequence identity to the VH CDRs 1, 2, and 3 of SEQ ID NO: 45. In some embodiments, the full set of VH CDRs 1, 2, and 3 (combined) in the CD3-binding VH region has at least 80% (such as, e.g., 80%, 85%, 90%, 95%, 98%, 99%, at least 85%, at least 90%, at least 95%, at least 98%, at least 99%) sequence identity to the VH CDRs 1, 2, and 3 of SEQ ID NO: 46. In some embodiments, the full set of VH CDRs 1, 2, and 3 (combined) in the CD3-binding VH region has at least 80% (such as, e.g., 80%, 85%, 90%, 95%, 985, 99%, at least 85%, at least 90%, at least 95%, at least 98%, at least 99%) sequence identity to the VH CDRs 1, 2, and 3 of SEQ ID NO: 47. In some embodiments, the full set of VH CDRs 1, 2, and 3 (combined) in the CD3-binding VH region has at least 80% (such as, e.g., 80%, 85%, 90%, 95%, 98%, 99% at least 85%, at least 90%, at least 95%, at least 98%, at least 99%) sequence identity to the VH CDRs 1, 2, and 3 of SEQ ID NO: 48.

In some embodiments, the CD3-binding VH region comprises the VH CDR1, VH CDR2, and VH CDR3 of any one of SEQ ID NOs: 31-48. In some embodiments, the CD3-binding VH region comprises the VH CDR1, VH CDR2, and VH CDR3 of SEQ ID NO: 31. In some embodiments, the CD3-binding VH region comprises the VH CDR1, VH CDR2, and VH CDR3 of SEQ ID NO: 32. In some embodiments, the CD3-binding VH region comprises the VH CDR1, VH CDR2, and VH CDR3 of SEQ ID NO: 33. In some embodiments, the CD3-binding VH region comprises the VH CDR1, VH CDR2, and VH CDR3 of SEQ ID NO: 34. In some embodiments, the CD3-binding VH region comprises the VH CDR1, VH CDR2, and VH CDR3 of SEQ ID NO: 35. In some embodiments, the CD3-binding VH region comprises the VH CDR1, VH CDR2, and VH CDR3 of SEQ ID NO: 36. In some embodiments, the CD3-binding VH region comprises the VH CDR1, VH CDR2, and VH CDR3 of SEQ ID NO: 37. In some embodiments, the CD3-binding VH region comprises the VH CDR1, VH CDR2, and VH CDR3 of SEQ ID NO: 38. In some embodiments, the CD3-binding VH region comprises the VH CDR1, VH CDR2, and VH CDR3 of SEQ ID NO: 39. In some embodiments, the CD3-binding VH region comprises the VH CDR1, VH CDR2, and VH CDR3 of SEQ ID NO: 40. In some embodiments, the CD3-binding VH region comprises the VH CDR1, VH CDR2, and VH CDR3 of SEQ ID NO: 41. In some embodiments, the CD3-binding VH region comprises the VH CDR1, VH CDR2, and VH CDR3 of SEQ ID NO: 42. In some embodiments, the CD3-binding VH region comprises the VH CDR1, VH CDR2, and VH CDR3 of SEQ ID NO: 43. In some embodiments, the CD3-binding VH region comprises the VH CDR1, VH CDR2, and VH CDR3 of SEQ ID NO: 44. In some embodiments, the CD3-binding VH region comprises the VH CDR1, VH CDR2, and VH CDR3 of SEQ ID NO: 45. In some embodiments, the CD3-binding VH region comprises the VH CDR1, VH CDR2, and VH CDR3 of SEQ ID NO: 46. In some embodiments, the CD3-binding VH region comprises the VH CDR1, VH CDR2, and VH CDR3 of SEQ ID NO: 47. In some embodiments, the CD3-binding VH region comprises the VH CDR1, VH CDR2, and VH CDR3 of SEQ ID NO: 48.

In some embodiments, the CD3-binding VH region comprises:
(a) a VH CDR1, a VH CDR2, and a VH CDR3 comprising the sequences of SEQ ID NOs: 20, 26, and 27, respectively;
(b) a VH CDR1, a VH CDR2, and a VH CDR3 comprising the sequences of SEQ ID NOs: 20, 26, and 28, respectively;
(c) a VH CDR1, a VH CDR2, and a VH CDR3 comprising the sequences of SEQ ID NOs: 20, 26, and 29, respectively;
(d) a VH CDR1, a VH CDR2, and a VH CDR3 comprising the sequences of SEQ ID NOs: 21, 26, and 28, respectively;
(e) a VH CDR1, a VH CDR2, and a VH CDR3 comprising the sequences of SEQ ID NOs: 22, 26, and 28, respectively;
(f) a VH CDR1, a VH CDR2, and a VH CDR3 comprising the sequences of SEQ ID NOs: 23, 26, and 28, respectively;
(g) a VH CDR1, a VH CDR2, and a VH CDR3 comprising the sequences of SEQ ID NOs: 24, 26, and 28, respectively;
(h) a VH CDR1, a VH CDR2, and a VH CDR3 comprising the sequences of SEQ ID NOs: 20, 26, and 30, respectively;
(i) a VH CDR1, a VH CDR2, and a VH CDR3 comprising the sequences of SEQ ID NOs: 25, 26, and 29, respectively; or
(j) a VH CDR1, a VH CDR2, and a VH CDR3 comprising the sequences of SEQ ID NOs: 24, 26, and 29, respectively.

In some embodiments, the CD3-binding VH region comprises a VH CDR1, a VH CDR2, and a VH CDR3 comprising the sequences of SEQ ID NOs: 20, 26, and 27, respectively. In some embodiments, the CD3-binding VH region comprises a VH CDR1, a VH CDR2, and a VH CDR3 comprising the sequences of SEQ ID NOs: 20, 26, and 28, respectively. In some embodiments, the CD3-binding VH region comprises a VH CDR1, a VH CDR2, and a VH CDR3 comprising the sequences of SEQ ID NOs: 20, 26, and 29, respectively. In some embodiments, the CD3-binding VH region comprises a VH CDR1, a VH CDR2, and a VH CDR3 comprising the sequences of SEQ ID NOs: 21, 26, and 28, respectively. In some embodiments, the CD3-binding VH region comprises a VH CDR1, a VH CDR2, and a VH CDR3 comprising the sequences of SEQ ID NOs: 22, 26, and 28, respectively. In some embodiments, the CD3-binding VH region comprises a VH CDR1, a VH CDR2, and a VH CDR3 comprising the sequences of SEQ ID NOs: 23, 26, and 28, respectively. In some embodiments, the CD3-binding VH region comprises a VH CDR1, a VH CDR2, and a VH CDR3 comprising the sequences of SEQ ID NOs: 24, 26, and 28, respectively. In some embodiments, the CD3-binding VH region comprises a VH CDR1, a VH CDR2, and a VH CDR3 comprising the sequences of SEQ ID NOs: 20, 26, and 30, respectively. In some embodiments, the CD3-binding VH region comprises a VH CDR1, a VH CDR2, and a VH CDR3 comprising the sequences of SEQ ID NOs: 25, 26, and 29, respectively. In some embodiments, the CD3-binding VH region comprises a VH CDR1, a VH CDR2, and a VH CDR3 comprising the sequences of SEQ ID NOs: 24, 26, and 29, respectively.

In some embodiments, the CD3-binding VH region comprises:
(i) a VH complementarity determining region one (CDR1) comprising the sequence

(SEQ ID NO: 55)
G F T F X₁₁ X₁₂ Y A,

wherein X₁₁ is D, A, or H; and X₁₂ is D or N;
(ii) a VH CDR2 comprising the sequence ISWNSGSI (SEQ ID NO: 26); and
(iii) a VH CDR3 comprising the sequence

(SEQ ID NO: 56)
A K D S R G Y G X₁₃ Y X₁₄ X₁₅ G GA Y,

wherein X₁₃ is D or S; X₁₄ is R or S; and X₁₅ is L or R.

In some embodiments, the VH CDR1, VH CDR2, and VH CDR3 sequences in the CD3-binding VH region are present in a human VH framework.

In some embodiments, the CD3-binding VH region has at least 80% (such as, e.g., 80%, 85%, 90%, 95%, 98%, 99%, at least 85%, at least 90%, at least 95%, at least 98%, at least 99%) sequence identity to any one of SEQ ID NOs: 31-48. In some embodiments, the CD3-binding VH region has at least 85% (such as, e.g., 85%, 90%, 95%, 98%, 99%, at least 90%, at least 95%, at least 98%, at least 99%) sequence identity to any one of SEQ ID NOs: 31-48. In some embodiments, the CD3-binding VH region has at least 90% (such as, e.g., 90%, 95%, 98%, 99%, at least 95%, at least 98%, at least 99%) sequence identity to any one of SEQ ID NOs: 31-48. In some embodiments, the CD3-binding VH region has at least 95% sequence identity to any one of SEQ ID NOs: 31-48.

In some embodiments, the CD3-binding VH region has at least 80% (such as, e.g., 80%, 85%, 90%, 95%, 98%, 99%, at least 85%, at least 90%, at least 95%, at least 98%, at least 99%) sequence identity to SEQ ID NO: 31. In some embodiments, the CD3-binding VH region has at least 80% (such as, e.g., 80%, 85%, 90%, 95%, 98%, 99%, at least 85%, at least 90%, at least 95%, at least 98%, at least 99%) sequence identity to SEQ ID NO: 32. In some embodiments, the CD3-binding VH region has at least 80% (such as, e.g., 80%, 85%, 90%, 95%, 98%, 99%, at least 85%, at least 90%, at least 95%, at least 98%, at least 99%) sequence identity to SEQ ID NO: 33. In some embodiments, the CD3-binding VH region has at least 80% (such as, e.g., 80%, 85%, 90%, 95%, 98%, 99%, at least 85%, at least 90%, at least 95%, at least 98%, at least 99%) sequence identity to SEQ ID NO: 34. In some embodiments, the CD3-binding VH region has at least 80% (such as, e.g., 80%, 85%, 90%, 95%, 98%, 99%, at least 85%, at least 90%, at least 95%, at least 98%, at least 99%) sequence identity to SEQ ID NO: 35. In some embodiments, the CD3-binding VH region has at least 80% (such as, e.g., 80%, 85%, 90%, 95%, 98%, 99%, at least 85%, at least 90%, at least 95%, at least 98%, at least 99%) sequence identity to SEQ ID NO: 36. In some embodiments, the CD3-binding VH region has at least 80% (such as, e.g., 80%, 85%, 90%, 95%, 98%, 99%, at least 85%, at least 90%, at least 95%, at least 98%, at least 99%) sequence identity to SEQ ID NO: 37. In some embodiments, the CD3-binding VH region has at least 80% (such as, e.g., 80%, 85%, 90%, 95%, 98%, 99%, at least 85%, at least 90%, at least 95%, at least 98%, at least 99%) sequence identity to SEQ ID NO: 38. In some embodiments, the CD3-binding VH region has at least 80% (such as, e.g., 80%, 85%, 90%, 95%, 98%, 99%, at least 85%, at least 90%, at least 95%, at least 98%, at least 99%) sequence identity to SEQ ID NO: 39. In some embodiments, the CD3-binding VH region has at least 80% (such as, e.g., 80%, 85%, 90%, 95%, 98%, 99%, at least 85%, at least 90%, at least 95%, at least 98%, at least 99%) sequence identity to SEQ ID NO: 40. In some embodiments, the CD3-binding VH region has at least 80% (such as, e.g., 80%, 85%, 90%, 95%, 98%, 99%, at least 85%, at least 90%, at least 95%, at least 98%, at least 99%) sequence identity to SEQ ID NO: 41. In some embodiments, the CD3-binding VH region has at least 80% (such as, e.g., 80%, 85%, 90%, 95%, 98%, 99%, at least 85%, at least 90%, at least 95%, at least 98%, at least 99%) sequence identity to SEQ ID NO: 42. In some embodiments, the CD3-binding VH region has at least 80% (such as, e.g., 80%, 85%, 90%, 95%, 98%, 99%, at least 85%, at least 90%, at least 95%, at least 98%, at least 99%) sequence identity to SEQ ID NO: 43. In some embodiments, the CD3-binding VH region has at least 80% (such as, e.g., 80%, 85%, 90%, 95%, 98%, 99%, at least 85%, at least 90%, at least 95%, at least 98%, at least 99%) sequence identity to SEQ ID NO: 44. In some embodiments, the CD3-binding VH region has at least 80% (such as, e.g., 80%, 85%, 90%, 95%, 98%, 99%, at least 85%, at least 90%, at least 95%, at least 98%, at least 99%) sequence identity to SEQ ID NO: 45. In some embodiments, the CD3-binding VH region has at least 80% (such as, e.g., 80%, 85%, 90%, 95%, 98%, 99%, at least 85%, at least 90%, at least 95%, at least 98%, at least 99%) sequence identity to SEQ ID NO: 46. In some embodiments, the CD3-binding VH region has at least 80% (such as, e.g., 80%, 85%, 90%, 95%, 98%, 99%, at least 85%, at least 90%, at least 95%, at least 98%, at least 99%) sequence identity to SEQ ID NO: 47. In some embodiments, the CD3-binding VH region has at least 80% (such as, e.g., 80%, 85%, 90%, 95%, 98%, 99%, at least 85%, at least 90%, at least 95%, at least 98%, at least 99%) sequence identity to SEQ ID NO: 48.

In some embodiments, the light chain variable region comprises the VL CDR1, VL CDR2, and VL CDR3 of SEQ ID NO: 52. In some embodiments, the light chain variable region comprises a VL CDR1, a VL CDR2, and a VL CDR3 comprising the sequence of SEQ ID NOs: 49, 50, and 51, respectively. In some embodiments, the VL CDR1, VL CDR2, and VL CDR3 sequences are present in a human VH framework.

In some embodiments, the light chain variable region has at least 80% (such as, e.g., 80%, 85%, 90%, 95%, 98%, 99%, at least 85%, at least 90%, at least 95%, at least 98%, at least 99%) sequence identity to SEQ ID NO: 52. In some embodiments, the light chain variable region has at least 85% (such as, e.g., 85%, 90%, 95%, 98%, 99%, at least 90%, at least 95%, at least 98%, at least 99%) sequence identity to SEQ ID NO: 52. In some embodiments, the light chain variable region has at least 90% (such as, e.g., 90%, 95%, 98%, 99%, at least 95%, at least 98%, at least 99%) sequence identity to SEQ ID NO: 52. In some embodiments, the light chain variable region has at least 95% sequence identity to SEQ ID NO: 52.

In some embodiments, the c-Kit binding protein is an anti-c-Kit antibody or fragment thereof.

In some embodiments, the anti-c-Kit antibody is a monoclonal antibody or fragment thereof. In some embodiments, the anti-c-Kit antibody is an isolated monoclonal antibody or fragment thereof.

In some embodiments, the anti-c-Kit antibody is an intact IgG molecule. In some embodiments, the anti-c-Kit antibody is an intact IgG1 molecule. In some embodiments, the anti-c-Kit antibody is an intact IgG2 molecule. In some embodiments, the anti-c-Kit antibody is an intact IgG4 molecule.

In some embodiments, the c-Kit binding protein is an antibody fragment. In some embodiments, the antibody fragment is an immunologically active portion of an intact IgG molecule. In some embodiments, the antibody fragment is an immunologically active portion of an intact IgG1 molecule. In some embodiments, the antibody fragment is an immunologically active portion of an intact IgG2 molecule. In some embodiments, the antibody fragment is an immunologically active portion of an intact IgG4 molecule. In some embodiments, the antibody fragment is a heavy chain-only antibody. In some embodiments, the antibody fragment is a TCA.

In some embodiments, the anti-c-Kit antibody or fragment thereof further comprises a Fc region. In some embodiments, the anti-c-Kit antibody or fragment thereof further comprises a variant Fc region. In some embodiments, the variant Fc region possesses at least about 80% homology (e.g., at least about 85%, at least about 90%, at least about 95%, at least about 98%, at least about 99%) with a native sequence Fc region.

In some embodiments, the variant Fc region comprises heterodimerizing alterations. In some embodiments, the heterodimerizing alterations comprise knob and holes substitutions (such as, e.g., in a variant IgG1 Fc region, 1) Y407T in one chain and T366Y in the other; 2) Y407A in one chain and T366W in the other; 3) F405A in one chain and T394W in the other; 4) F405W in one chain and T394S in the other; 5) Y407T in one chain and T366Y in the other; 6) T366Y and F405A in one chain and T394W and Y407T in the other; 7) T366W and F405W in one chain and T394S and Y407A in the other; 8) F405W and Y407A in one chain and T366W and T394S in the other; or 9) T366W in one polypeptide of the Fc and T366S, L368A, and Y407V in the other). In some embodiments, the heterodimerizing alterations comprise substitutions that create new disulfide bridges (such as, e.g., in a variant IgG1 Fc region, 1) Y349C in one Fc polypeptide chain and S354C in the other; 2) Y349C in one Fc polypeptide chain and E356C in the other; 3) Y349C in one Fe polypeptide chain and E357C in the other; 4) L351C in one Fc polypeptide chain and S354C in the other; 5) T394C in one Fc polypeptide chain and E397C in the other; or 6) D399C in one Fc polypeptide chain and K392C in the other). In some embodiments, the heterodimerizing alterations comprise charge pair substitutions (such as, e.g., 1) K409E in one chain plus D399K in the other; 2) K409E in one chain plus D399R in the other; 3) K409D in one chain plus D399K in the other; 4) K409D in one chain plus D399R in the other; 5) K392E in one chain plus D399R in the other; 6) K392E in one chain plus D399K in the other; 7) K392D in one chain plus D399R in the other; 8) K392D in one chain plus D399K in the other; 9) K409D and K360D in one chain plus D399K and E356K in the other; 10) K409D and K370D in one chain plus D399K and E357K in the other; 11) K409D and K392D in one chain plus D399K, E356K, and E357K in the other; 12) K409D and K392D on one chain and D399K on the other; 13) K409D and K392D on one chain plus D399K and E356K on the other; 14) K409D and K392D on one chain plus D399K and D357K on the other; 15) K409D and K370D on one chain plus D399K and D357K on the other; 16) D399K on one chain plus K409D and K360D on the other; or 17) K409D and K439D on one chain plus D399K and E356K on the other).

In some embodiments, the Fc region is a silenced Fc region. In some embodiments, the silenced Fc region comprises substitution of one or more (such as, e.g., two or more) of Fc region residues 238, 265, 269, 270, 297, 327 and 329 according to EU numbering. In some embodiments, the silenced Fc region comprises a substitution that alters glycosylation. In some embodiments, the silenced Fc region comprises an effector-less mutation (such as, e.g., an N297A, an N297G, a DANA mutation (D265A+N297A), or a DANG mutation (D265A+N297G) in the CH2 region). In some embodiments, the silenced Fc region comprises K322A and L234A/L235A mutations.

In some embodiments, an anti-c-Kit antibody or fragment thereof further comprises a heavy chain constant region sequence in the absence of a CH1 sequence. In some embodiments, the anti-c-Kit antibody or fragment thereof comprises a heavy chain constant region comprising a hinge region, a CH2 domain, and a CH3 domain. In some embodiments, the hinge region comprises a wild type human IgG4 hinge region sequence (SEQ ID NO: 61). In some embodiments, the hinge region comprises a variant human IgG4 hinge region sequence comprising an S228P mutation (SEQ ID NO: 62). In some embodiments, the CH2 domain comprises a wild type human IgG4 CH2 domain sequence (SEQ ID NO: 63). In some embodiments, the CH2 domain comprises a variant human IgG4 CH2 domain comprising an F234A mutation, an L235A mutation, or both an F234A mutation and an L235A mutation. In some embodiments, the CH3 domain comprises a wild type human IgG4 CH3 domain sequence (SEQ ID NO: 65). In some embodiments, the CH3 domain comprises a variant human IgG4 CH3 domain sequence comprising a T366W mutation. In some embodiments, the CH3 domain comprises a variant human IgG4 CH3 domain sequence comprising a T366S, an L368A mutation, and a Y407V mutation.

Table S10 provides the sequences of human IgG1 and IgG4 Fc region sequences, as well as versions of these sequences that incorporate additional mutations (variants) that may impart additional desired properties to anti-c-Kit antibodies and fragments thereof described herein. Table S11 provides example human c-Kit, human CD3 epsilon, and human CD3 delta sequences.

TABLE S10
Human IgG1 and IgG4 Fc Region Sequences and Variants Thereof

| Polypeptide Name | Amino Acid Sequence |
|---|---|
| Human IgG1 | ASTKGPSVFPLAPSSKSTSGGTAALGCLVKDYFPEPVTVS |
| (UniProt No. | WNSGALTSGVHTFPAVLQSSGLYSLSSVVTVPSSSLGTQT |
| P01857) | YICNVNHKPSNTKVDKKVEPKSCDKTHTCPPCPAPELLGG |
| | PSVFLFPPKPKDTLMISRTPEVTCVVVDVSHEDPEVKFNW |
| | YVDGVEVHNAKTKPREEQYNSTYRVVSVLTVLHQDWLNGK |
| | EYKCKVSNKALPAPIEKTISKAKGQPREPQVYTLPPSRDE |
| | LTKNQVSLTCLVKGFYPSDIAVEWESNGQPENNYKTTPPV |
| | LDSDGSFFLYSKLTVDKSRWQQGNVFSCSVMHEALHNHYT |
| | QKSLSLSPGK (SEQ ID NO: 57) |
| Human IgG4 | ASTKGPSVFPLAPCSRSTSESTAALGCLVKDYFPEPVTVS |
| (UniProt No. | WNSGALTSGVHTFPAVLQSSGLYSLSSVVTVPSSSLGTKT |
| P01861) | YTCNVDHKPSNTKVDKRVESKYGPPCPSCPAPEFLGGPSV |
| | FLFPPKPKDTLMISRTPEVTCVVVDVSQEDPEVQFNWYVD |
| | GVEVHNAKTKPREEQFNSTYRVVSVLTVLHQDWLNGKEYK |
| | CKVSNKGLPSSIEKTISKAKGQPREPQVYTLPPSQEEMTK |
| | NQVSLTCLVKGFYPSDIAVEWESNGQPENNYKTTPPVLDS |
| | DGSFFLYSRLTVDKSRWQEGNVFSCSVMHEALHNHYTQKS |
| | LSLSLGK (SEQ ID NO: 58) |
| Human IgG1 with | ASTKGPSVFPLAPSSKSTSGGTAALGCLVKDYFPEPVTVSWN |
| silencing mutations | SGALTSGVHTFPAVLQSSGLYSLSSVVTVPSSSLGTQTYICNV |
| (Fc region) | NHKPSNTKVDKKVEPKSCDKTHTCPPCPAPEAAGGPSVFLFP |
| | PKPKDTLMISRTPEVTCVVVDVSHEDPEVKFNWYVDGVEVH |
| | NAKTKPREEQYNSTYRVVSVLTVLHQDWLNGKEYKCKVSN |
| | KALPAPIEKTISKAKGQPREPQVYTLPPSREEMTKNQVSLTCL |
| | VKGFYPSDIAVEWESNGQPENNYKTTPPVLDSDGSFFLYSKL |
| | TVDKSRWQQGNVFSCSVMHEALHNHYTQKSLSLSPGK (SEQ |
| | ID NO: 59) |
| Human IgG4 with | ASTKGPSVFPLAPCSRSTSESTAALGCLVKDYFPEPVTVSWNS |
| silencing mutations | GALTSGVHTFPAVLQSSGLYSLSSVVTVPSSSLGTKTYTCNV |
| (Fc region) | DHKPSNTKVDKRVESKYGPPCPPCPAPEAAGGPSVFLFPPKP |
| | KDTLMISRTPEVTCVVVDVSQEDPEVQFNWYVDGVEVHNA |
| | KTKPREEQFNSTYRVVSVLTVLHQDWLNGKEYKCKVSNKGL |
| | PSSIEKTISKAKGQPREPQVYTLPPSQEEMTKNQVSLTCLVKG |
| | FYPSDIAVEWESNGQPENNYKTTPPVLDSDGSFFLYSRLTVD |
| | KSRWQEGNVFSCSVMHEALHNHYTQKSLSLSLGK |
| | (SEQ ID NO: 60) |
| Human IgG4 hinge | ESKYGPPCPSCPA (SEQ ID NO: 61) |
| region (wild type) | |
| Human IgG4 hinge | ESKYGPPCPPCPA (SEQ ID NO: 62) |
| region (S228P) | |
| Human IgG4 CH2 | APEFLGGPSVFLFPPKPKDTLMISRTPEVTCVVVDVSQEDPEV |
| domain sequence | QFNWYVDGVEVHNAKTKPREEQFNSTYRVVSVLTVLHQDW |
| (wild type) | LNGKEYKCKVSNKGLPSSIEKTISKAK (SEQ ID NO: 63) |
| Human IgG4 CH2 | APEAAGGPSVFLFPPKPKDTLMISRTPEVTCVVVDVSQEDPEV |
| domain sequence | QFNWYVDGVEVHNAKTKPREEQFNSTYRVVSVLTVLHQDW |
| (F234A, L235A) | LNGKEYKCKVSNKGLPSSIEKTISKAK (SEQ ID NO: 64) |
| Human IgG4 CH3 | GQPREPQVYTLPPSQEEMTKNQVSLTCLVKGFYPSDIAVEWE |
| domain sequence | SNGQPENNYKTTPPVLDSDGSFFLYSRLTVDKSRWQEGNVFS |
| (wild type) | CSVMHEALHNHYTQKSLSLSLGK (SEQ ID NO: 65) |
| Human IgG4 CH3 | GQPREPQVYTLPPSQEEMTKNQVSLWCLVKGFYPSDIAVEW |
| domain sequence | ESNGQPENNYKTTPPVLDSDGSFFLYSRLTVDKSRWQEGNVF |
| (knob, T366W) | SCSVMHEALHNHYTQKSLSLSLGK (SEQ ID NO: 66) |
| Human IgG4 CH3 | GQPREPQVYTLPPSQEEMTKNQVSLSCAVKGFYPSDIAVEWE |
| domain sequence | SNGQPENNYKTTPPVLDSDGSFFLVSRLTVDKSRWQEGNVFS |
| (hole, T366S, L368A, | CSVMHEALHNHYTQKSLSLSLGK (SEQ ID NO: 67) |
| Y407V) | |

TABLE S11
Example Human c-Kit, CD3 Epsilon, and CD3 Delta Sequences

| Polypeptide Name | Amino Acid Sequence |
|---|---|
| Human c-Kit | MRGARGAWDFLCVLLLLLRVQTGSSQPSVSPGEPSPPSIHPG |
| (UniProt P10721) | KSDLIVRVGDEIRLLCTDPGFVKWTFEILDETNENKQNEWITE |
| | KAEATNTGKYTCTNKHGLSNSIYVFVRDPAKLFLVDRSLYG |
| | KEDNDTLVRCPLTDPEVTNYSLKGCQGKPLPKDLRFIPDPKA |
| | GIMIKSVKRAYHRLCLHCSVDQEGKSVLSEKFILKVRPAFKA |
| | VPVVSVSKASYLLREGEEFTVTCTIKDVSSSVYSTWKRENSQ |
| | TKLQEKYNSWHHGDFNYERQATLTISSARVNDSGVFMCYAN |
| | NTFGSANVTTTLEVVDKGFINIFPMINTTVFVNDGENVDLIVE |
| | YEAFPKPEHQQWIYMNRTFTDKWEDYPKSENESNIRYVSELH |
| | LTRLKGTEGGTYTFLVSNSDVNAAIAFNVYVNTKPEILTYDR |
| | LVNGMLQCVAAGFPEPTIDWYFCPGTEQRCSASVLPVDVQT |
| | LNSSGPPFGKLVVQSSIDSSAFKHNGTVECKAYNDVGKTSAY |
| | FNFAFKGNNKEQIHPHTLFTPLLIGFVIVAGMMCIIVMILTYK |
| | YLQKPMYEVQWKVVEEINGNNYVYIDPTQLPYDHKWEFPR |
| | NRLSFGKTLGAGAFGKVVEATAYGLIKSDAAMTVAVKMLK |
| | PSAHLTEREALMSELKVLSYLGNHMNIVNLLGACTIGGPTLVI |
| | TEYCCYGDLLNFLRRKRDSFICSKQEDHAEAALYKNLLHSKE |
| | SSCSDSTNEYMDMKPGVSYVVPTKADKRRSVRIGSYIERDVT |
| | PAIMEDDELALDLEDLLSFSYQVAKGMAFLASKNCIHRDLAA |
| | RNILLTHGRITKICDFGLARDIKNDSNYVVKGNARLPVKWMA |
| | PESIFNCVYTFESDVWSYGIFLWELFSLGSSPYPGMPVDSKFY |
| | KMIKEGFRMLSPEHAPAEMYDIMKTCWDADPLKRPTFKQIV |
| | QLIEKQISESTNHIYSNLANCSPNRQKPVVDHSVRINSVGSTAS |
| | SSQPLLVHDDV (SEQ ID NO: 19) |
| Example extracellular | QDGNEEMGGITQTPYKVSISGTTVILTCPQYPGSEILWQHNDK |
| domain of human | NIGGDEDDKNIGSDEDHLSLKEFSELEQSGYYVCYPRGSKPE |
| CD3 epsilon | DANFYLYLRARVCENCMEMD (SEQ ID NO: 68) |
| Human CD3 epsilon | MQSGTHWRVLGLCLLSVGVWGQDGNEEMGGITQTPYKVSIS |
| | GTTVILTCPQYPGSEILWQHNDKNIGGDEDDKNIGSDEDHLSL |
| | KEFSELEQSGYYVCYPRGSKPEDANFYLYLRARVCENCMEM |
| | DVMSVATIVIVDICITGGLLLLVYYWSKNRKAKAKPVTRGAG |
| | AGGRQRGQNKERPPPVPNPDYEPIRKGQRDLYSGLNQRRI |
| | (SEQ ID NO: 69) |
| Human CD3 delta | MEHSTFLSGLVLATLLSQVSPFKIPIEELEDRVFVNCNTSITWV |
| | EGTVGTLLSDITRLDLGKRILDPRGIYRCNGTDIYKDKESTVQ |
| | VHYRMCQSCVELDPATVAGIIVTDVIATLLLALGVFCFAGHE |
| | TGRLSGAADTQALLRNDQVYQPLRDRDDAQYSHLGGNWAR |
| | NK (SEQ ID NO: 70) |

Some embodiments of the present disclosure relate to a polynucleotide encoding a single domain antibody that specifically binds to c-Kit as described herein.

Some embodiments of the present disclosure relate to a composition comprising one or more polynucleotide(s) encoding a c-Kit binding protein as described herein. In some embodiments, the c-Kit binding protein is an anti-c-Kit antibody or fragment thereof.

Some embodiments of the present disclosure relate to a recombinant expression vector comprising a single domain antibody that specifically binds to c-Kit as described herein, as well as a host cell comprising the recombinant expression vector.

Some embodiments of the present disclosure relate to one or more recombinant expression vector(s) comprising one or more polynucleotide(s) encoding a c-Kit binding protein as described herein, as well as a host cell comprising the one or more recombinant expression vector(s).

Some embodiments of the present disclosure relate to a chimeric antigen receptor comprising a single domain antibody that specifically binds to c-Kit as described herein, as well as an immune cell expressing the chimeric antigen receptor.

A CAR-expressing immune cell (e.g., a CAR-expressing T cell; a CAR-expressing NK cell) of the present disclosure typically comprises: an extracellular antigen-binding domain which specifically binds to c-Kit, wherein the extracellular antigen-binding domain comprises a single domain antibody disclosed herein; a transmembrane domain; and an intracellular signaling domain. In some embodiments, the intracellular signaling domain comprises a costimulatory domain and/or a primary signaling domain.

In some embodiments, a CAR-expressing immune cell of the present disclosure comprises a transmembrane domain that comprises a transmembrane domain of a protein, such as, e.g., a protein chosen from the alpha, beta or zeta chain of the T-cell receptor, CD28, CD3 epsilon, CD45, CD4, CDS, CD8, CD9, CD16, CD22, CD33, CD37, CD64, CD80, CD86, CD134, CD137, and CD154. In some embodiments, first domain (i.e., the antigen binding domain) is connected to the transmembrane domain by a hinge region.

In some embodiments, the costimulatory domain is a functional signaling domain from a protein, such as, e.g., a protein chosen from a MHC class I molecule, a TNF receptor protein, an Immunoglobulin-like protein, a cytokine receptor, an integrin, a signaling lymphocytic activation molecule (SLAM protein), an activating NK cell receptor, BTLA, a Toll ligand receptor, OX40, CD2, CD7, CD27, CD28, CD30, CD40, CDS, ICAM-1, LFA-1 (CD11a/CD18), 4-1BB (CD137), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83.

In some embodiments, the primary signaling domain comprises a functional signaling domain of CD3 zeta.

In some embodiments, the intracellular signaling domain comprises a functional signaling domain of 4-1BB and/or a functional signaling domain of CD3 zeta. In some embodiments, the intracellular signaling domain comprises a functional signaling domain of CD27 and/or a functional signaling domain of CD3 zeta. In some embodiments, the intracellular signaling domain comprises a functional signaling domain of CD28 and/or a functional signaling domain of CD3 zeta. In some embodiments, the intracellular signaling domain comprises a functional signaling domain of ICOS and/or a functional signaling domain of CD3 zeta.

In some embodiments, the CAR further comprises a leader sequence.

In some embodiments, the CAR further comprises a hinge between the extracellular antigen binding domain and the transmembrane domain. In some embodiments, the hinge comprises a sequence derived from a human CD8a, IgG4, and/or CD4 sequence. In some embodiments, the hinge comprises a sequence derived from a human CD8a sequence. In some embodiments, the hinge comprises an amino acid sequence comprising a human CD8a amino acid sequence comprising TTTPAPRPPTPAPTIASQPLSLRPEACRPAAGGAVHTRGLDFACD (SEQ ID NO: 72) or a sequence having at least about 70%, at least about 80%, at least about 90%, at least about 95%, or at least about 99% identity to the amino acid sequence comprising SEQ ID NO: 72. In some embodiments, the hinge is encoded by a nucleic acid sequence comprising actaccacaccagcacctagaccaccaactccagctccaaccatcgcgagtcagcccctgagtctgagacctgaggcctgcaggcc agctgcaggaggagctgtgcacaccaggggcctggacttcgcctgcgac (SEQ ID NO: 73). In some embodiments, the hinge is encoded by a nucleic acid sequence comprising

(SEQ ID NO: 74)
ACCACAACCCCTGCCCCCAGACCTCCCACACCCGCCCCTACCATCGCGA
GTCAGCCCCTGAGTCTGAGACCTGAGGCCTGCAGGCCAGCTGCAGGAGG
AGCTGTGCACACCAGGGGCCTGGACTTCGCCTGCGAC.

A CAR-expressing immune cell (e.g., a CAR-expressing T cell; a CAR-expressing NK cell) of the present disclosure may be allogenic or autologous and may be prepared according to standard techniques of the art.

Some embodiments of the present disclosure relate to producing a c-Kit binding protein (such as, e.g., an anti-c-Kit antibody or fragment thereof) described herein, comprising growing a cell described herein under conditions permissive for expression of the antibody, and isolating the c-Kit binding protein (such as, e.g., the anti-c-Kit antibody or fragment thereof) from the cell.

Some embodiments of the present disclosure relate to an antibody-drug conjugate comprising a single domain antibody that specifically binds to c-Kit, as described herein. In some embodiments, the drug in the antibody-drug conjugate is a chemotherapy agent. In some embodiments, the drug in the antibody-drug conjugate is a radionuclide. In some embodiments, the antibody-drug conjugate is for use in a diagnostic application, such as, e.g., the detection or monitoring of a disease associated with c-Kit expression, such as, e.g., a cancer.

Some embodiments of the present disclosure relate to a pharmaceutical composition comprising a c-Kit binding protein, antibody-drug conjugate, anti-c-Kit antibody, antibody fragment, or CAR-expressing immune cell and a pharmaceutically acceptable excipient.

Non-limiting examples of pharmaceutically acceptable excipients include adjuvants, solid carriers, water, buffers, or other carriers used in the art to hold therapeutic components, or combinations thereof.

Pharmaceutical compositions of the present disclosure may be prepared for storage by mixing proteins having the desired degree of purity with optional pharmaceutically acceptable carriers, excipients or stabilizers (see, e.g. Remington's Pharmaceutical Sciences 16th edition, Osol, A. Ed. (1980)), such as, e.g., in the form of lyophilized formulations or aqueous solutions. Acceptable carriers, excipients, or stabilizers are nontoxic to recipients at the dosages and concentrations employed, and include, but are not limited to, buffers such as phosphate, citrate, and other organic acids; antioxidants including ascorbic acid and methionine; preservatives (such as, e.g., octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™ PLURONICS™ or polyethylene glycol (PEG).

In some embodiments, the pharmaceutical composition may comprise formulation materials for modifying, maintaining or preserving, for example, the pH, osmolarity, viscosity, clarity, color, isotonicity, odor, sterility, stability, rate of dissolution or release, adsorption or penetration of the composition. In such embodiments, suitable formulation materials include, but are not limited to, amino acids (such as, e.g., glycine, glutamine, asparagine, arginine or lysine); antimicrobials; antioxidants (such as, e.g., ascorbic acid, sodium sulfite or sodium hydrogen-sulfite); buffers (such as, e.g., borate, bicarbonate, Tris-HCl, citrates, phosphates or other organic acids); bulking agents (such as, e.g., mannitol or glycine); chelating agents (such as, e.g., ethylenediamine tetraacetic acid (EDTA)); complexing agents (such as, e.g., caffeine, polyvinylpyrrolidone, beta-cyclodextrin or hydroxypropyl-beta-cyclodextrin); fillers; monosaccharides; disaccharides; and other carbohydrates (such as, e.g., glucose, mannose or dextrins); proteins (such as, e.g., serum albumin, gelatin or immunoglobulins); coloring, flavoring and diluting agents; emulsifying agents; hydrophilic polymers (such as, e.g., polyvinylpyrrolidone); low molecular weight polypeptides; salt-forming counterions (such as, e.g., sodium); preservatives (such as, e.g., benzalkonium chloride, benzoic acid, salicylic acid, thimerosal, phenethyl alcohol, methylparaben, propylparaben, chlorhexidine, sorbic acid or hydrogen peroxide); solvents (such as, e.g., glycerin, propylene glycol or polyethylene glycol); sugar alcohols (such as, e.g., mannitol or sorbitol); suspending agents; surfactants or wetting agents (such as, e.g., pluronics, PEG, sorbitan esters, polysorbates such as, e.g., polysorbate 20, polysorbate 80, triton, tromethamine, lecithin, cholesterol, tyloxapal); stability enhancing agents (such as, e.g., sucrose or sorbitol); tonicity enhancing agents (such as, e.g., alkali metal halides, such as, e.g., sodium or potassium chloride, mannitol, sorbitol); delivery vehicles; diluents; excipients and/or pharmaceutical adjuvants. Methods and suitable materials for formulating molecules for therapeutic use are known in the pharmaceutical arts, and are described, for example, in REMINGTON'S PHARMACEUTICAL SCIENCES, 18th Edition, (A. R. Genrmo, ed.), 1990, Mack Publishing Company. Pharmaceutical compositions of the present disclosure include, but are not limited to, liquid, frozen, and lyophilized compositions.

Pharmaceutical compositions for parenteral administration are preferably sterile and substantially isotonic and manufactured under Good Manufacturing Practice (GMP) conditions. Pharmaceutical compositions can be provided in unit dosage form (such as, e.g., the dosage for a single administration). The formulation depends on the route of administration chosen. The c-Kit binding proteins (such as, e.g., anti-c-Kit antibodies and fragments thereof) and antibody-drug conjugates described herein can be administered by intravenous injection or infusion or subcutaneously. For injection administration, the c-Kit binding proteins (such as, e.g., anti-c-Kit antibodies and fragments thereof) and antibody-drug conjugates described herein can be formulated in aqueous solutions, preferably in physiologically-compatible buffers to reduce discomfort at the site of injection. The solution can contain carriers, excipients, or stabilizers as discussed above. Alternatively, c-Kit binding proteins (such as, e.g., anti-c-Kit antibodies and fragments thereof) and antibody-drug conjugates described herein can be in lyophilized form for reconstitution with a suitable vehicle, e.g., sterile pyrogen-free water, before use. The lyophilized material may be reconstituted in, e.g., bacteriostatic water for injection (BWFI), physiological saline, phosphate buffered saline (PBS), or the same formulation the protein had been in prior to lyophilization.

Antibody formulations are disclosed, for example, in U.S. Pat. No. 9,034,324. Similar formulations can be used for anti-c-Kit antibodies and fragments thereof described herein. Subcutaneous antibody formulations are described, for example, in US20160355591 and US20160166689, the content of each of which is incorporated herein by reference in its entirety.

Some embodiments of the present disclosure relate to a method of treating a disease associated with c-Kit expression in a subject in need thereof comprising administering to the subject a therapeutically effective dose of at least one c-Kit binding protein, antibody-drug conjugate, anti-c-Kit antibody, antibody fragment, or CAR-expressing immune cell as described herein.

In some embodiments, the administration results in slowing or inhibition of tumor growth or metastasis of a c-Kit-expressing cancer. Measurement of the reduction of the growth of tumor cells can be determined by multiple different methodologies that are well known in the art. Non-limiting examples include direct measurement of tumor dimension, measurement of excised tumor mass and comparison to control subjects, measurement via imaging techniques (such as, e.g., CT or MRI) that may or may not use isotopes or luminescent molecules (such as, e.g., luciferase) for enhanced analysis, and the like. In some embodiments, administration of the at least one c-Kit binding protein, antibody-drug conjugate, anti-c-Kit antibody, antibody fragment, or CAR-expressing immune cell results in a reduction of in vivo growth of tumor cells as compared to a control antigen binding agent by at least about 10%, 20%, 30%, 40%, 50%, 60%, 70%, 80%, 90%, or 100%, with an about 100% reduction in tumor growth indicating a complete response and disappearance of the tumor. In some embodiments, administration of the at least one c-Kit binding protein, antibody-drug conjugate, anti-c-Kit antibody, antibody fragment, or CAR-expressing immune cell results in a reduction of in vivo growth of tumor cells as compared to a control antigen binding agent by about 50-100%, about 75-100%, or about 90-100%. In some embodiments, administration of the at least one c-Kit binding protein, antibody-drug conjugate, anti-c-Kit antibody, antibody fragment, or CAR-expressing immune cell results in a reduction of in vivo growth of tumor cells as compared to a control antigen binding agent by about 50-60%, about 60-70%, about 70-80%, about 80-90%, or about 90-100%.

Effective doses for the treatment of disease vary depending upon many different factors, including means of administration, target site, physiological state of the patient, whether the patient is human or an animal, other medications administered, and whether treatment is prophylactic or therapeutic. Usually, the patient is a human, but nonhuman mammals may also be treated, e.g., companion animals such as dogs, cats, horses, etc., laboratory mammals such as rabbits, mice, rats, etc., and the like. Treatment dosages can be titrated to optimize safety and efficacy.

Dosage levels can be readily determined by the ordinarily skilled clinician, and can be modified as required, e.g., as required to modify a subject's response to therapy. The amount of active ingredient that can be combined with the carrier materials to produce a single dosage form varies depending upon the host treated and the particular mode of administration.

In some embodiments, the c-Kit binding protein, antibody-drug conjugate, anti-c-Kit antibody, antibody fragment, or CAR-expressing immune cell is administered to the subject parenterally. Parenteral administration refers to administration of the molecule by routes other than through the gastrointestinal tract and can include intraperitoneal, intramuscular, intravenous, intraarterial, intradermal, subcutaneous, intracerebral, intracerebroventricular, and intrathecal administration.

In some embodiments, the c-Kit binding protein, antibody-drug conjugate, anti-c-Kit antibody, antibody fragment, or CAR-expressing immune cell is administered to the subject intravenously.

Parenteral or intravenous administration can be performed by injection (e.g. using a needle and a syringe) or by infusion (e.g. via a catheter and a pump system). It is envisaged that the administration according to the present disclosure is via intravenous injection or via intravenous infusion. Usually, an intravenous (IV) infusion is administered via a line, a port or a catheter (small, flexible tube), such as a central venous access or a central venous catheter (CVC), which is a catheter placed into a large vein, or a peripheral venous catheter (PVC), which is a catheter placed into a peripheral vein. In general, catheters or lines can be placed in veins in the neck (internal jugular vein), chest (subclavian vein or axillary vein), groin (femoral vein), or through veins in the arms (also known as a PICC line, or peripherally inserted central catheters). Central IV lines have catheters that are advanced through a vein and empty into a large central vein, usually the superior vena cava, inferior vena cava or even the right atrium of the heart. A peripheral intravenous (PIV) line is used on peripheral veins (the veins in the arms, hands, legs and feet). A port is a central venous line that does not have an external connector; instead, it has a small reservoir that is covered with silicone rubber and is implanted under the skin. Medication is administered intermittently by placing a small needle through the skin, piercing the silicone, into the reservoir. When the needle is withdrawn, the reservoir cover reseals itself. The cover can accept hundreds of needle sticks during its lifetime.

In some embodiments, the disease associated with c-Kit expression is a cancer.

In some embodiments, the cancer is chosen from small cell lung cancer (SCLC), gastrointestinal stromal tumors (GISTs), melanoma, and acute myeloid leukemia (AML).

Administration of a c-Kit binding protein, antibody-drug conjugate, anti-c-Kit antibody, antibody fragment, or CAR-expressing immune cell as described herein can also be accompanied by administration of other anti-cancer agents or therapeutic treatments (such as surgical resection of a tumor). Any suitable anti-cancer agent can be administered in combination with the c-Kit binding proteins, antibody-drug conjugates, anti-c-Kit antibodies, antibody fragments, or CAR-expressing immune cells disclosed herein. Example anti-cancer agents include, but are not limited to, chemotherapeutic agents, such as, e.g., mitotic inhibitors, alkylating agents, anti-metabolites, intercalating antibiotics, growth factor inhibitors, cell cycle inhibitors, enzymes, topoisomerase inhibitors, anti-survival agents, biological response modifiers, anti-hormones (such as, e.g. anti-androgens) and anti-angiogenesis agents. Other anti-cancer treatments include radiation therapy and other antibodies that specifically target cancer cells.

In some embodiments, the c-Kit binding protein, antibody-drug conjugate, anti-c-Kit antibody, antibody fragment, or CAR-expressing immune cell is administered before, during, or after surgery.

Some embodiments of the present disclosure relate to a c-Kit binding protein, antibody-drug conjugate, anti-c-Kit antibody, antibody fragment, or CAR-expressing immune cell as described herein for use in the treatment of a disease associated with c-Kit expression. In some embodiments, the disease associated with c-Kit expression is a cancer. In some embodiments, the cancer is chosen from small cell lung cancer (SCLC), gastrointestinal stromal tumors (GISTs), melanoma, and acute myeloid leukemia (AML).

Some embodiments of the present disclosure relate to a use of a c-Kit binding protein, antibody-drug conjugate, anti-c-Kit antibody, antibody fragment, or CAR-expressing immune cell as described herein in the manufacture of a medicament for the treatment of a disease associated with c-Kit expression. In some embodiments, the disease associated with c-Kit expression is a cancer. In some embodiments, the cancer is chosen from small cell lung cancer (SCLC), gastrointestinal stromal tumors (GISTs), melanoma, and acute myeloid leukemia (AML).

Some embodiments relate to a method of preconditioning a subject prior to a stem cell transplant, the method comprising administering to the subject a therapeutically effective dose of at least one c-Kit binding protein, antibody-drug conjugate, anti-c-Kit antibody, antibody fragment, or CAR-expressing immune cell as described herein. Additionally, some embodiments relate to a c-Kit binding protein, antibody-drug conjugate, anti-c-Kit antibody, antibody fragment, or CAR-expressing immune cell as described herein for use in a method of preconditioning a subject prior to a stem cell transplant. In addition, some embodiments relate to a use of a c-Kit binding protein, antibody-drug conjugate, anti-c-Kit antibody, antibody fragment, or CAR-expressing immune cell as described herein in the manufacture of a medicament for use in a method of preconditioning a subject prior to a stem cell transplant.

In some embodiments, the subject is suffering from a condition in which a stem cell transplant is considered to be beneficial, such as, e.g., a hematologic disease or a hematological malignancy, such as, e.g., myelodysplastic syndrome or leukemia. In some embodiments, the subject is suffering from acute myeloid leukemia.

In some embodiments, the c-Kit binding protein, antibody-drug conjugate, anti-c-Kit antibody, antibody fragment, or CAR-expressing immune cell is administered before or during the stem cell transplant.

In some embodiments, the stem cell transplant is an autologous stem cell transplant (ASCT). In some embodiments, the method reduces the number of stem cells, progenitor cells, and/or cancer stem cells in the subject.

In some embodiments, the stem cell transplant is a hematopoietic stem cell transplant (HCST). In some embodiments, the HCST is an autologous, allogeneic, syngeneic, or xenogeneic HSCT. In some embodiments, the method reduces the number of hematopoietic stem cells, hematopoietic progenitor cells, and/or hematopoietic cancer stem cells in the subject.

In some embodiments, the preconditioning does not further comprise radiation therapy and/or busulfan therapy. In some embodiments, the method replaces a preconditioning regimen comprising radiation therapy and/or busulfan therapy.

Dosage levels can be readily determined by the ordinarily skilled clinician, and can be modified as required, e.g., as required to modify a subject's response to therapy. The amount of active ingredient that can be combined with the carrier materials to produce a single dosage form varies depending upon the host treated and the particular mode of administration.

In some embodiments, the c-Kit binding protein, antibody-drug conjugate, anti-c-Kit antibody, antibody fragment, or CAR-expressing immune cell is administered to the subject parenterally. Parenteral administration refers to administration of the molecule by routes other than through the gastrointestinal tract and can include intraperitoneal, intramuscular, intravenous, intraarterial, intradermal, subcutaneous, intracerebral, intracerebroventricular, and intrathecal administration.

In some embodiments, the c-Kit binding protein, antibody-drug conjugate, anti-c-Kit antibody, antibody fragment, or CAR-expressing immune cell is administered to the subject intravenously.

Some embodiments of the present disclosure relate to a method of confirming the diagnosis of cancer in a subject by contacting a sample from the subject diagnosed with cancer with a single domain antibody or c-Kit binding protein described herein, and detecting binding of the single domain antibody or c-Kit binding protein to the sample. An increase in binding of the single domain antibody or c-Kit binding protein to the sample relative to binding of the single domain antibody or c-Kit binding protein to a control sample confirms the cancer diagnosis. In some embodiments, the method further includes contacting a detection antibody that specifically recognizes the single domain antibody or c-Kit binding protein with the sample, and detecting binding of the detection antibody.

Some embodiments of the present disclosure relate to a method of detecting a cancer associated with c-Kit expression in a subject. The method includes contacting a sample from the subject with a single domain antibody or c-Kit binding protein described herein, and detecting binding of the single domain antibody or c-Kit binding protein to the sample. An increase in binding of the single domain antibody or c-Kit binding protein to the sample relative to a control sample detects cancer in the subject. In some embodiments, the methods further comprise contacting a detection antibody that specifically recognizes the single domain antibody or c-Kit binding protein with the sample, and detecting binding of the detection antibody.

Some embodiments of the present disclosure relate to a kit comprising a c-Kit binding protein, antibody-drug conjugate, anti-c-Kit antibody, antibody fragment, or CAR-expressing immune cell as described herein or a pharmaceutical composition comprising the same and instructions for use. The kit can further contain a least one additional reagent, such as, e.g. a chemotherapeutic drug, etc. Kits typically include a label indicating the intended use of the contents of the kit. The term “label” as used herein includes any writing, or recorded material supplied on or with a kit, or which otherwise accompanies a kit.

In some embodiments, the kit is a diagnostic kit. In some embodiments, a kit is provided for detecting c-Kit in a biological sample, such as, e.g., a blood sample or tissue sample. For example, to confirm a cancer diagnosis in a subject, a biopsy can be performed to obtain a tissue sample for histological examination. Alternatively, a blood sample can be obtained to detect the presence of soluble c-Kit protein or fragment. Kits for detecting a polypeptide will typically comprise a single domain antibody or c-Kit binding protein according to the present disclosure. In some embodiments, the single domain antibody or c-Kit binding protein is labeled (such as, e.g., with a fluorescent, radioactive, or an enzymatic label).

In some embodiments, the kit may additionally comprise means for detecting a label (such as, e.g., enzyme substrates for enzymatic labels, filter sets to detect fluorescent labels, appropriate secondary labels such as a secondary antibody, or the like). The kits may additionally include buffers and other reagents routinely used for the practice of a particular method. Such kits and appropriate contents are well known to those of skill in the art.

### EXAMPLES

In order that the present disclosure may be more fully understood, the following examples are set forth. It should be understood that these examples are for illustrative purposes only and are not to be construed as limiting this disclosure in any manner.

### Example 1. Binding to c-Kit-Expressing Cell Line and Off-Target Cell Line

Cell binding for example anti-c-Kit single domain antibodies of the disclosure was assessed using SEM cells expressing human c-Kit (GNNK+ isoform or GNNK− isoform), NOMO-1 cells expressing c-Kit, and K562 cells that do not express c-Kit protein. Pelleted cells were resuspended in flow buffer (lx phosphate buffered saline (PBS), 1% bovine serum albumin (BSA), and 0.1% NaN₃) at 1.25×10⁶ cells/mL. Antibody-containing supernatants were diluted 1:5 in flow buffer and 40 μL cells plus 10 μL diluted antibody supernatant were incubated for 30 minutes at 4° C. Cells were washed with flow buffer and resuspended in 50 μL phycoerythrin (PE)-conjugated secondary antibody (Southern Biotech #2042-09) diluted in flow buffer to 1.25 μg/mL and incubated for 20 minutes at 4° C. After two wash steps, cells were resuspended in flow buffer and analyzed using a Guava easyCyte 8HT system. Table E1 summarizes the target binding activity of the anti-c-Kit single domain antibodies as fold over background MFI signal.

TABLE E1
Binding to c-Kit-Expressing Cell Lines and Off-Target Cell Line

| | SEM | SEM | | |
|---|---|---|---|---|
| Clone_ID # | (GNNK+) | (GNNK−) | NOMO-1 | K562 |
| 408366 | 945.1 | 1756.7 | 43.2 | 2.0 |
| 407921 | 813.6 | 1748.0 | 30.5 | 2.3 |
| 407922 | 829.3 | 1740.5 | 31.1 | 2.5 |
| 408389 | 933.9 | 1794.4 | 43.9 | 2.1 |
| 408198 | 388.2 | 102.8 | 45.7 | 1.8 |

### Example 2. Binding to c-Kit-Expressing Cell Lines and Off-Target Cell Line

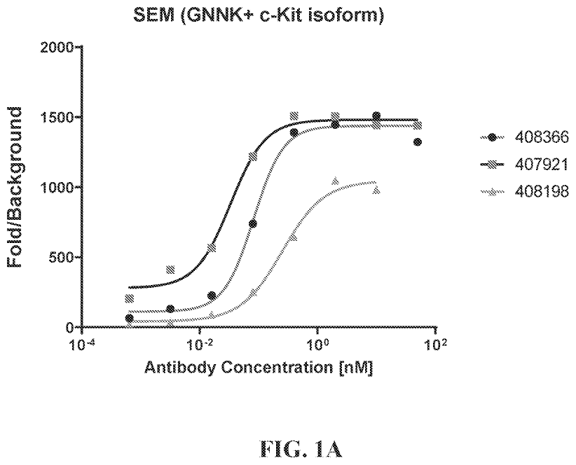

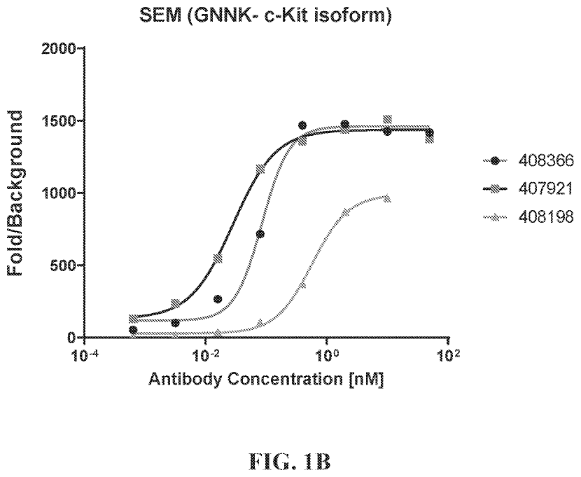

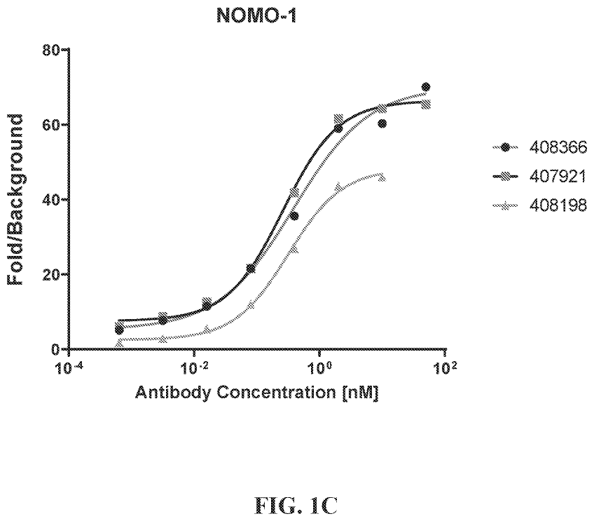

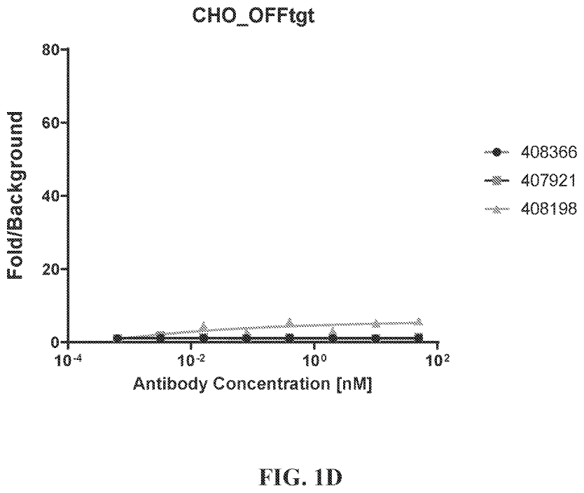

Cell-binding dose curves for example anti-c-Kit single domain antibodies described herein were performed using SEM cells expressing human c-Kit (GNNK+ isoform (FIG. 1A) and GNNK− isoform (FIG. 1B)), NOMO-1 cells expressing human c-Kit (FIG. 1C), and CHO-OFFtgt cells that do not express c-Kit protein (FIG. 1D). The single domain antibodies were tested at a starting concentration of 50 nM, followed by 5-fold serial dilutions for an 8-point dose curve. All washes and dilutions of cells, antibodies, and reagents were performed using flow buffer (lx phosphate buffered saline (PBS), 1% bovine serum albumin (BSA), and 0.1% NaN₃). Cells were pelleted and resuspended at 1×10⁶ cells/mL in flow buffer. Then, 50 μL cells were combined with 50 μL of test antibody and incubated 30 minutes at 4° C., followed by two wash steps with flow buffer. Cells were then resuspended in 50 μL PE-conjugated secondary diluted to 1.25 μg/mL (Southern Biotech #2042-09) and incubated for 20 minutes at 4° C. After two wash steps, cells were resuspended in flow buffer and analyzed using a BD Celesta system. PE mean fluorescence intensity was plotted as a fold over background (cells incubated with secondary detection antibody only).

### Example 3. Cell Binding EC50 Values on c-Kit-Expressing Cell Lines

To determine cell binding EC50 values for example anti-c-Kit single domain antibodies of the present disclosure, cell binding dose curves were performed on SEM cells expressing human GNNK+c-Kit isoform, SEM cells expressing human GNNK− c-Kit isoform, and NOMO-1 cells expressing human c-Kit. The single domain antibodies were tested at a starting dose of 50 nM, followed by 5-fold serial dilutions for an 8-point dose curve as described in Example 2. The transformed data were plotted as an xy-graph using the non-linear regression curve fit (available in GraphPad Prism 8.4.3) to obtain the EC50 values (nM), which are summarized in Table E2.

TABLE E2
Cell Binding EC50 Values on c-Kit-Expressing Cell Lines

| | SEM | SEM | |
|---|---|---|---|
| Clone_ID # | (GNNK+) | (GNNK−) | NOMO-1 |
| 408366 | 0.08 | 0.09 | 0.39 |
| 407921 | 0.03 | 0.03 | 0.26 |
| 408198 | 0.25 | 0.58 | 0.32 |

### Example 4. Binding Affinity and Epitope Binning

Table E3 summarizes affinity and epitope bin information for example anti-c-Kit single domain antibodies of the present disclosure. The affinity of each single domain antibody to recombinant human c-Kit (Acro Biosystems, #CD7-H52H4) was measured by Bio-layer interferometry (BLI) using an Octet HTX. AHC (Anti-hIgG Fc Capture) sensors were used to immobilize test antibody at 5 μg/mL. After baseline readings, sensors were dipped into antigen solutions (starting at 500 nM followed by a 7-point, 2-fold dilution series). Association and dissociation were measured for 180 and 240 seconds, respectively. Data were analyzed with Octet Data Analysis v11.0 HT (ForteBio) using a standard 1:1 binding model. Epitope bins for the single domain antibodies were determined by an in-tandem competition BLI binding experiment using an Octet HTX. The antigen (5 μg/mL) was loaded on the sensor Ni-NTA sensor, followed by a baseline in kinetic buffer. The antigen coated sensor was then dipped into a saturating concentration Antibody 1. A second baseline was then set for 300 seconds. The antigen-Antibody 1 complex was dipped into Antibody 2 solution for 180 seconds and then allowed to dissociate.

TABLE E3
Binding Affinity and Epitope Binning

| Clone_ID # | KD (M) | Epitope Bin |
|---|---|---|
| 408366 | 4.71E−09 | 1 |
| 407921 | 2.77E−09 | 1 |
| 408198 | 7.28E−09 | 2 |

### Example 5. CAR-T Mediated Anti-Tumor Cytotoxicity In Vitro

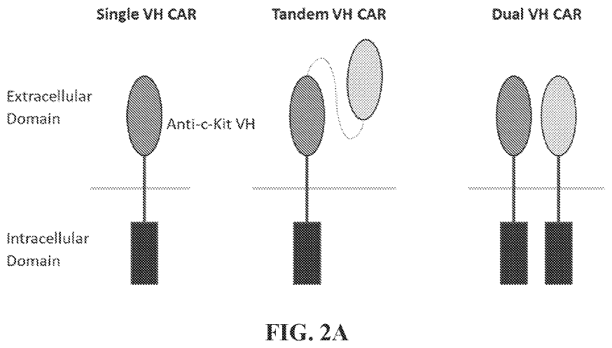

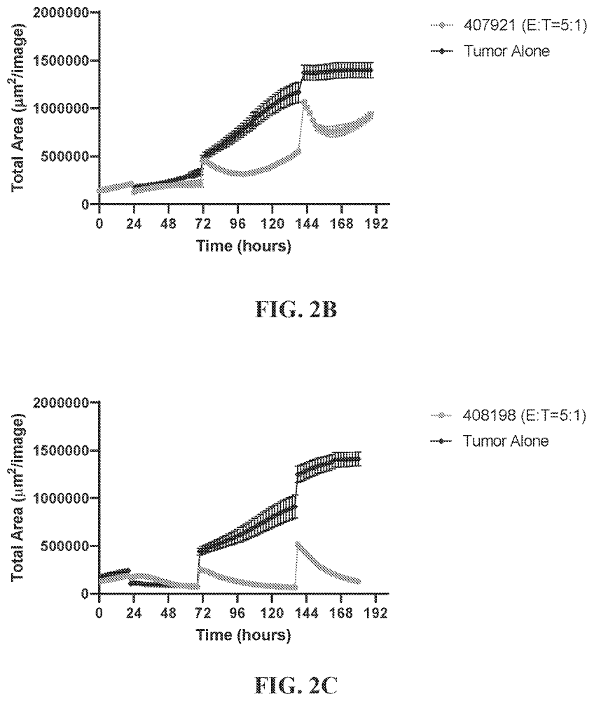

CAR-T cell in vitro cytotoxicity was assessed by a serial killing assay of anti-c-Kit CAR-T cells cocultured with the Nomo-1 AML tumor cell line. CAR-T cells were generated from healthy donor T cells by delivery, via electroporation, of (1) an anti-c-kit CAR-containing DNA transgene flanked by piggyBac inverted terminal repeats (ITR) and (2) an mRNA encoding the Super piggyBac transposase, which mediates permanent integration of the ITR flanked DNA transgene into the host cell genome. A schematic illustration of an example CAR-T structure comprising a single anti-c-Kit extracellular binding domain comprising a single domain antibody sequence of the present disclosure is shown in FIG. 2A.

Nomo-1 cells endogenously express c-Kit and were engineered to express GFP to enable tracking of cell killing by fluorescence imaging. Tumor cells were plated at 20,000 cells per well in a flat-bottom tissue culture treated 96-well plate and either cultured alone (tumor alone) or cocultured with anti-c-kit CAR-T cells at an effector-to-target (E:T) ratio of 5:1. Cells were maintained in incubators at 37° C. with 5% 02 and GFP fluorescent signal was read every 2 hours for up to 188 hours using the Incucyte® S3 System (Sartorius, Gottingen, Germany). Anti-c-Kit CAR-T cells were rechallenged by addition of fresh tumor cells at 24, 72, and 144 hours. Results are shown in FIG. 2B and FIG. 2C. Anti-c-Kit CAR-T cells expressing a c-Kit extracellular binding domain comprising a single domain antibody sequence of the present disclosure effectively slowed tumor growth over multiple rechallenges in vitro as shown by reduced fluorescent signal in cocultures containing anti-c-Kit CAR-T cells compared to tumor alone controls.

### Example 6. CAR-T Cell Mediated Anti-Tumor Cytotoxicity In Vivo

CAR-T cell in vivo cytotoxicity was assessed by treating tumor engrafted mice using anti-c-Kit CAR-T cells. CAR-T cells were generated from healthy donor T cells by delivery, via electroporation, of (1) an anti-c-kit CAR-containing DNA transgene flanked by piggyBac inverted terminal repeats (ITR) and (2) an mRNA encoding the Super piggyBac transposase, which mediates permanent integration of the ITR flanked DNA transgene into the host cell genome. NSG mice were inoculated with 1×10⁶ Nomo-1 tumor cells, a human AML cell line, which endogenously expresses c-Kit and was engineered to express CBG-luciferase to enable tumor tracking by bioluminescent imaging (BLI). On day 7 post-tumor injection, mice were randomized into groups based on tumor burden and were treated with anti-c-kit CAR-T cells, control CAR-T cells targeting an antigen not expressed on Nomo-1 cells, or no CAR-T cells. Tumor burden was tracked over time by BLI.

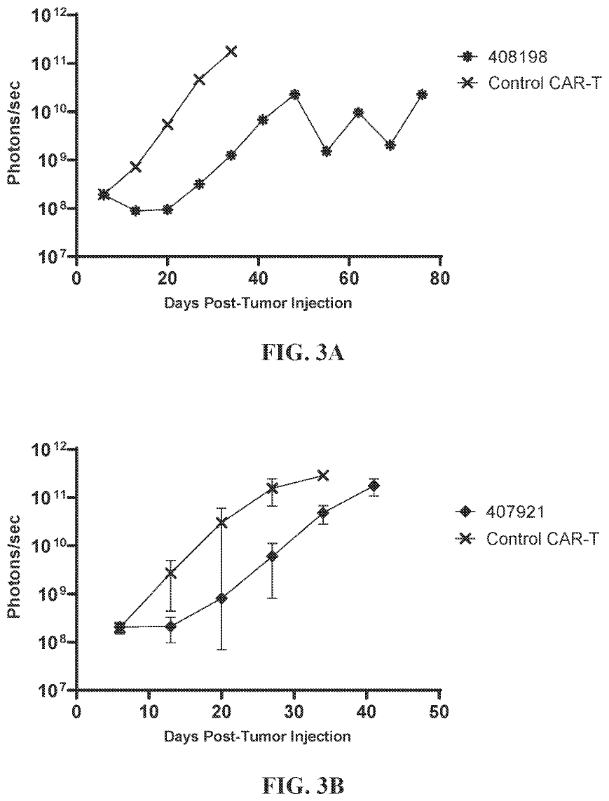

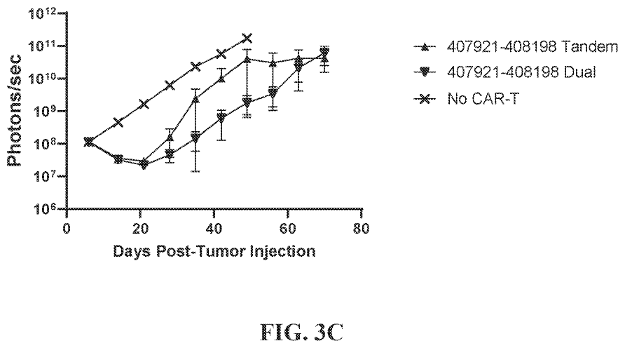

Anti-c-Kit CAR-T cells expressing a c-Kit extracellular binding domain comprising a single domain antibody sequence of the present disclosure effectively slowed tumor growth in vivo (FIGS. 3A-3C). In vivo cytotoxicity was maintained whether the extracellular binding domain comprised a single VH sequence (FIGS. 3A, 3B) or two VH sequences displayed in tandem or dual configuration (FIG. 3C). FIG. 2A is a schematic illustration of example CAR-T structures of various configurations, each comprising anti-c-Kit extracellular binding domain(s) comprising single domain antibody sequence(s) of the present disclosure.

All documents, or portions of documents, cited in this application, including but not limited to patents, patent applications, articles, books, and treatises, are hereby expressly incorporated by reference. What is described in an embodiment of the disclosure can be combined with one or more other embodiments of the disclosure unless context clearly indicates otherwise.

The disclosed subject matter is not intended to be limited in scope by the specific embodiments described herein, which are instead intended as non-limiting illustrations of individual aspects of the disclosure. Functionally equivalent methods and components are within the scope of the disclosure. Indeed, various modifications of the disclosed subject matter, in addition to those shown and described herein, will be apparent to those skilled in the art from the foregoing description and accompanying drawing(s). Such modifications are intended to fall within the scope of the disclosed subject matter.

The descriptions of the various embodiments and/or examples of the disclosed subject matter have been presented for purposes of illustration, but are not intended to be exhaustive or limiting in any way. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, and/or to enable others of ordinary skill in the art to understand the disclosed subject matter.

What is claimed is:

1. A single domain antibody which specifically binds to c-Kit, wherein the single domain antibody comprises a heavy chain variable (VH) region in which the full set of VH CDRs 1, 2, and 3 (combined) has at least 95% sequence identity to the VH CDRs 1, 2, and 3 of any one of SEQ ID NOs: 14-18.

2. A single domain antibody which specifically binds to c-Kit, wherein the single domain antibody comprises a heavy chain variable (VH) region comprising:
(a) (i) a VH complementarity determining region one (CDR1) comprising a sequence having at most two amino acid modifications relative to SEQ ID NO: 1, SEQ ID NO: 2, or SEQ ID NO: 3;
(ii) a VH CDR2 comprising a sequence having at most two amino acid modifications relative to SEQ ID NO: 4, SEQ ID NO: 5, SEQ ID NO: 6, or SEQ ID NO: 7; and
(iii) a VH CDR3 comprising a sequence having at most two amino acid modifications relative to SEQ ID NO: 8, SEQ ID NO: 9, or SEQ ID NO: 10; or
(b) a VH CDR1 comprising a sequence having at most two amino acid modifications relative to SEQ ID NO: 11; a VH CDR2 comprising a sequence having at most two amino acid modifications relative to SEQ ID NO: 12; and a VH CDR3 comprising a sequence having at most two amino acid modifications relative to SEQ ID NO: 13.

3. A single domain antibody which specifically binds to c-Kit, wherein the single domain antibody comprises a heavy chain variable (VH) region comprising:
(i) a VH complementarity determining region one (CDR1) comprising the sequence
(SEQ ID NO: 53)
G X₁ T X₂ X₃ X₄ Y A,
wherein X₁ is F or L; X₂ is F or I; X₃ is D or S; and X₄ is S or T;
(ii) a VH CDR2 comprising the sequence
(SEQ ID NO: 71)
I S X₅ X₆ G X₇ X₈ T,
wherein X₅ is V or T, X₆ is R or G; X₇ is G or S; and X₈ is S or R; and
(iii) a VH CDR3 comprising the sequence
(SEQ ID NO: 54)
A T G Y D X₉ S G X₁₀ Y Y G G F D Y,
wherein X₉ is S or P; and X₁₀ is Y or H.

4. A single domain antibody which specifically binds to c-Kit, wherein the single domain antibody comprises a heavy chain variable (VH) region comprising:
(i) a VH complementarity determining region one (CDR1) comprising a sequence chosen from SEQ ID NO: 1, SEQ ID NO: 2, and SEQ ID NO: 3;
(ii) a VH CDR2 comprising a sequence chosen from SEQ ID NO: 4, SEQ ID NO: 5, SEQ ID NO: 6, and SEQ ID NO: 7; and
(iii) a VH CDR3 comprising a sequence chosen from SEQ ID NO: 8, SEQ ID NO: 9, and SEQ ID NO: 10.

5. A single domain antibody which specifically binds to c-Kit, wherein the single domain antibody comprises a heavy chain variable (VH) region comprising:
(a) a VH CDR1, a VH CDR2, and a VH CDR3 comprising the sequences of SEQ ID NOs: 1, 4, and 8, respectively;
(b) a VH CDR1, a VH CDR2, and a VH CDR3 comprising the sequences of SEQ ID NOs: 2, 5, and 9, respectively;
(c) a VH CDR1, a VH CDR2, and a VH CDR3 comprising the sequences of SEQ ID NOs: 3, 6, and 8, respectively;
(d) a VH CDR1, a VH CDR2, and a VH CDR3 comprising the sequences of SEQ ID NOs: 2, 7, and 10, respectively; or
(e) a VH CDR1, a VH CDR2, and a VH CDR3 comprising the sequences of SEQ ID NOs: 11, 12, and 13, respectively.

6. A single domain antibody which specifically binds to c-Kit, wherein the single domain antibody comprises a heavy chain variable (VH) region comprising the VH CDR1, VH CDR2, and VH CDR3 of any one of SEQ ID NOs: 14-18.

7. The single domain antibody according to any one of claims 1 to 6, wherein the VH CDR1, VH CDR2, and VH CDR3 sequences are present in a human VH framework.

8. A single domain antibody which specifically binds to c-Kit, wherein the single domain antibody comprises a heavy chain variable (VH) region having at least 95% sequence identity to any one of SEQ ID NOs: 14-18.

9. A single domain antibody which specifically binds to c-Kit, wherein the single domain antibody comprises a heavy chain variable (VH) region chosen from SEQ ID NOs: 14-18.

10. The single domain antibody according to any one of claims 1 to 9, wherein the single domain antibody specifically binds to human c-Kit.

11. The single domain antibody according to any one of claims 1 to 10, wherein the single domain antibody binds to human c-Kit with a K_{D} of from about 10⁻⁹ M to about 10⁻⁶ M.

12. The single domain antibody according to any one of claims 1 to 11, wherein the single domain antibody is an isolated single domain antibody.

13. A c-Kit binding protein comprising the single domain antibody according to any one of claims 1 to 11.

14. The c-Kit binding protein according to claim 13, wherein the c-Kit binding protein specifically binds to human c-Kit.

15. The c-Kit binding protein according to claim 13 or 14, wherein the c-Kit binding protein further specifically binds to CD3.

16. The c-Kit binding protein according to any one of claims 13 to 15, wherein the c-Kit binding protein further specifically binds to human CD3.

17. The c-Kit binding protein according to any one of claims 13 to 16, wherein the c-Kit binding protein is an isolated monoclonal antibody.

18. An antibody-drug conjugate comprising the single domain antibody according to any one of claims 1 to 11.

19. An anti-c-Kit antibody comprising the single domain antibody according to any one of claims 1 to 11.

20. The anti-c-Kit antibody according to claim 19, wherein the anti-c-Kit antibody is multi-specific.

21. The anti-c-Kit antibody according to claim 19 or 20, wherein the anti-c-Kit antibody further specifically binds to a tumor-specific antigen other than c-Kit.

22. The anti-c-Kit antibody according to any one of claims 19 to 21, wherein the anti-c-Kit antibody is bispecific.

23. The anti-c-Kit antibody according to any one of claims 19 to 22, wherein the anti-c-Kit antibody further specifically binds to CD3.

24. The anti-c-Kit antibody according to any one of claims 19 to 23, wherein the anti-c-Kit antibody further specifically binds to human CD3.

25. The anti-c-Kit antibody according to any one of claims 19 to 24, wherein the anti-c-Kit antibody further comprises a CD3-binding heavy chain variable (VH) region.

26. The anti-c-Kit antibody according to any one of claims 19 to 25, wherein the anti-c-Kit antibody is of the IgG1 or IgG4 subtype.

27. The anti-c-Kit antibody according to any one of claims 19 to 26, wherein the anti-c-Kit antibody further comprises a CD3-binding heavy chain variable (VH) region that is paired with a light chain variable (VL) region.

28. The anti-c-Kit antibody according to claim 25 or 27, wherein the CD3-binding VH region comprises:
(i) a VH complementarity determining region one (CDR1) comprising a sequence having at most two amino acid modifications relative to any one of SEQ ID NOs: 20-25;
(ii) a VH CDR2 comprising a sequence having at most two amino acid modifications relative to SEQ ID NO: 26; and
(iii) a VH CDR3 comprising a sequence having at most two amino acid modifications relative to any one of SEQ ID NOs: 27-30.

29. The anti-c-Kit antibody according to claim 28, wherein the CD3-binding VH CDR1 comprises a sequence chosen from SEQ ID NOs: 20-25.

30. The anti-c-Kit antibody according to claim 28 or 29, wherein the CD3-binding VH CDR2 comprises the sequence of SEQ ID NO: 26.

31. The anti-c-Kit antibody according to any one of claims 28 to 30, wherein the CD3-binding VH CDR3 comprises a sequence chosen from SEQ ID NOs: 27-30.

32. The anti-c-Kit antibody according to any one of claim 25 or 27 to 31, wherein the full set of VH CDRs 1, 2, and 3 (combined) in the CD3-binding VH region has at least 95% sequence identity to the VH CDRs 1, 2, and 3 of any one of SEQ ID NOs: 31-48.

33. The anti-c-Kit antibody according to claim 25 or 27, wherein the CD3-binding VH region comprises:
(i) a VH complementarity determining region one (CDR1) comprising the sequence
(SEQ ID NO: 55)
G F T F X₁₁ X₁₂ Y A,
wherein X₁₁ is D, A, or H; and X₁₂ is D or N;
(ii) a VH CDR2 comprising the sequence ISWNSGSI (SEQ ID NO: 26); and
(iii) a VH CDR3 comprising the sequence
(SEQ ID NO: 56)
A K D S R G Y G X₁₃ Y X₁₄ X₁₅ G GA Y,
wherein X₁₃ is D or S; X₁₄ is R or S; and X₁₅ is L or R.

34. The anti-c-Kit antibody according to claim 25 or 27, wherein the CD3-binding VH region comprises the VH CDR1, VH CDR2, and VH CDR3 of any one of SEQ ID NOs: 31-48.

35. The anti-c-Kit antibody according to any one of claims 25 to 34, wherein the VH CDR1, VH CDR2, and VH CDR3 sequences in the CD3-binding VH region are present in a human VH framework.

36. The anti-c-Kit antibody according to any one of claims 25 to 35, wherein the CD3-binding VH region has at least 95% sequence identity to any one of SEQ ID NOs: 31-48.

37. The anti-c-Kit antibody according to any one of claims 25 to 36, wherein the CD3-binding VH region comprises:
(a) a VH CDR1, a VH CDR2, and a VH CDR3 comprising the sequences of SEQ ID NOs: 20, 26, and 27, respectively;
(b) a VH CDR1, a VH CDR2, and a VH CDR3 comprising the sequences of SEQ ID NOs: 20, 26, and 28, respectively;
(c) a VH CDR1, a VH CDR2, and a VH CDR3 comprising the sequences of SEQ ID NOs: 20, 26, and 29, respectively;
(d) a VH CDR1, a VH CDR2, and a VH CDR3 comprising the sequences of SEQ ID NOs: 21, 26, and 28, respectively;
(e) a VH CDR1, a VH CDR2, and a VH CDR3 comprising the sequences of SEQ ID NOs: 22, 26, and 28, respectively;
(f) a VH CDR1, a VH CDR2, and a VH CDR3 comprising the sequences of SEQ ID NOs: 23, 26, and 28, respectively;
(g) a VH CDR1, a VH CDR2, and a VH CDR3 comprising the sequences of SEQ ID NOs: 24, 26, and 28, respectively;
(h) a VH CDR1, a VH CDR2, and a VH CDR3 comprising the sequences of SEQ ID NOs: 20, 26, and 30, respectively;
(i) a VH CDR1, a VH CDR2, and a VH CDR3 comprising the sequences of SEQ ID NOs: 25, 26, and 29, respectively; or
(j) a VH CDR1, a VH CDR2, and a VH CDR3 comprising the sequences of SEQ ID NOs: 24, 26, and 29, respectively.

38. The anti-c-Kit antibody according to claim 37, wherein the CD3-binding VH region comprises a VH CDR1, a VH CDR2, and a VH CDR3 comprising the sequences of SEQ ID NOs: 20, 26, and 27, respectively.

39. The anti-c-Kit antibody according to any one of claims 27 to 38, wherein the light chain variable region comprises the VL CDR1, VL CDR2, and VL CDR3 of SEQ ID NO: 52.

40. The anti-c-Kit antibody according to any one of claims 27 to 39, wherein the light chain variable region comprises a VL CDR1, a VL CDR2, and a VL CDR3 comprising the sequences of SEQ ID NOs: 49, 50, and 51, respectively.

41. The anti-c-Kit antibody according to any one of claims 27 to 40, wherein the VL CDR1, VL CDR2, and VL CDR3 sequences are present in a human VH framework.

42. The anti-c-Kit antibody according to any one of claims 27 to 41, wherein the light chain variable region has at least 95% sequence identity to SEQ ID NO: 52.

43. The anti-c-Kit antibody according to any one of claims 19 to 42, wherein the anti-c-Kit antibody specifically binds to human c-Kit.

44. The anti-c-Kit antibody according to any one of claims 19 to 43, wherein the anti-c-Kit antibody is an isolated antibody.

45. An antibody fragment that specifically binds to c-Kit, wherein the antibody fragment comprises a fragment of the anti-c-Kit antibody according to any one of claims 19 to 44.

46. The antibody fragment according to claim 45, wherein the antibody fragment is a three-chain antibody-like molecule.

47. A chimeric antigen receptor (CAR)-expressing immune cell comprising a CAR that comprises an extracellular antigen-binding domain which specifically binds to c-Kit, wherein the extracellular antigen-binding domain comprises the single domain antibody according to any one of claims 1 to 11.

48. The CAR-expressing immune cell according to claim 47, wherein the single domain antibody comprises a heavy chain variable (VH) region comprising a VH CDR1, a VH CDR2, and a VH CDR3 comprising the sequences of SEQ ID NOs: 2, 5, and 9, respectively.

49. The CAR-expressing immune cell according to claim 47 or claim 48, wherein the single domain antibody comprises a heavy chain variable (VH) region of SEQ ID NO: 15.

50. The CAR-expressing immune cell according to claim 47, wherein the single domain antibody comprises a heavy chain variable (VH) region comprising a VH CDR1, a VH CDR2, and a VH CDR3 comprising the sequences of SEQ ID NOs: 11, 12, and 13, respectively.

51. The CAR-expressing immune cell according to claim 47 or claim 50, wherein the single domain antibody comprises a heavy chain variable (VH) region of SEQ ID NO: 18.

52. The CAR-expressing immune cell according to any one of claims 47 to 51, wherein the CAR further comprises a transmembrane domain and an intracellular signaling domain.

53. The CAR-expressing immune cell according to claim 52, wherein the CAR further comprises a hinge between the extracellular antigen binding domain and the transmembrane domain.

54. The CAR-expressing immune cell according to any one of claims 47 to 53, wherein the immune cell is chosen from T cells and natural killer (NK) cells.

55. A pharmaceutical composition comprising at least one c-Kit binding protein, antibody-drug conjugate, anti-c-Kit antibody, antibody fragment, or CAR-expressing immune cell according to any one of claims 13 to 54 and a pharmaceutically acceptable excipient.

56. A polynucleotide encoding:
the single domain antibody according to any one of claims 1 to 12; or
a chimeric antigen receptor comprising an extracellular antigen-binding domain which specifically binds to c-Kit, wherein the extracellular antigen-binding domain comprises the single domain antibody according to any one of claims 1 to 11.

57. A composition comprising one or more polynucleotide(s) encoding the c-Kit binding protein, anti-c-Kit antibody, or antibody fragment according to any one of claims 13 to 17 or 19 to 46.

58. A recombinant expression vector comprising the polynucleotide or composition according to claim 56 or claim 57.

59. A host cell comprising the recombinant expression vector according to claim 58.

60. A method of treating a disease associated with c-Kit expression in a subject in need thereof, comprising administering to the subject at least one c-Kit binding protein, antibody-drug conjugate, anti-c-Kit antibody, antibody fragment, or CAR-expressing immune cell according to any one of claims 13 to 54.

61. The method according to claim 60, wherein the disease associated with c-Kit expression is a cancer.

62. The method according to claim 60 or claim 61, wherein the disease associated with c-Kit expression is chosen from small cell lung cancer (SCLC), gastrointestinal stromal tumors (GISTs), melanoma, and acute myeloid leukemia (AML).

63. A method of preconditioning a subject prior to a stem cell transplant, comprising administering to the subject at least one c-Kit binding protein, antibody-drug conjugate, anti-c-Kit antibody, antibody fragment, or CAR expressing immune cell according to any one of claims 13 to 54.

64. The method according to claim 63, wherein the subject is suffering from a condition in which a stem cell transplant is considered to be beneficial.

65. The method according to claim 63 or claim 64, wherein the subject is suffering from myelodysplastic syndrome or leukemia.

66. The method according to any one of claims 63 to 65, wherein the stem cell transplant is an autologous stem cell transplant (ASCT).


SEQUENCE LISTING (74 sequences)

SEQ ID NO: 1 - protein, 8 aa, synthetic construct
GLTISTYA

SEQ ID NO: 2 - protein, 8 aa, synthetic construct
GFTFDSYA

SEQ ID NO: 3 - protein, 8 aa, synthetic construct
GLTISSYA

SEQ ID NO: 4 - protein, 8 aa, synthetic construct
ISTGGSST

SEQ ID NO: 5 - protein, 8 aa, synthetic construct
ISVRGGST

SEQ ID NO: 6 - protein, 8 aa, synthetic construct
ISTGGSRT

SEQ ID NO: 7 - protein, 8 aa, synthetic construct
ISTGGGST

SEQ ID NO: 8 - protein, 16 aa, synthetic construct
ATGYDSSGHYYGGFDY

SEQ ID NO: 9 - protein, 16 aa, synthetic construct
ATGYDPSGHYYGGFDY

SEQ ID NO: 10 - protein, 16 aa, synthetic construct
ATGYDSSGYYYGGFDY

SEQ ID NO: 11 - protein, 10 aa, synthetic construct
GGSISSSSYY

SEQ ID NO: 12 - protein, 7 aa, synthetic construct
IDYNGGT

SEQ ID NO: 13 - protein, 15 aa, synthetic construct
ARQNPRRYASGASDY

SEQ ID NO: 14 - protein, 123 aa, synthetic construct
EVQLLESGGGLVQPGGSLRLSCAASGLTISTYAMSWVRQAPGKGLEWVSAISTGGSSTYYADSVKGRFTISRDNSKNTLYLQMNSLRAEDTAVYYCATGYDSSGHYYGGFDYRGQGTLVTVSS

SEQ ID NO: 15 - protein, 123 aa, synthetic construct
EVQLLESGGGLVQPGGSLRLSCAASGFTFDSYAMSWVRQAPGKGLEWVSAISVRGGSTYYADSVKGRFTISRDNSKNTLYLQMNSLRAEDTAVYSCATGYDPSGHYYGGFDYRGQGTLVTVSS

SEQ ID NO: 16 - protein, 123 aa, synthetic construct
EVQLLESGGGLVQPGGSLRLSCAASGLTISSYAMSWVRQAPGKGLEWVSAISTGGSRTYYADSVKGRFTISRDNSKNTLYLQMNSLRAEDTAVYFCATGYDSSGHYYGGFDYRGQGTLVTVSS

SEQ ID NO: 17 - protein, 123 aa, synthetic construct
EVQLLESGGGLVQPGGSLRLSCAASGFTFDSYAMSWVRQAPGEGLEWVSAISTGGGSTYYADSVKGRFTISRDNSKNMLFLQMNSLRAEDTAVYSCATGYDSSGYYYGGFDYRGQGTLVTVSS

SEQ ID NO: 18 - protein, 123 aa, synthetic construct
QLQLQESGPGLVKPSETLSLTCTVSGGSISSSSYYWGWIRQPPGKGLEWIGSIDYNGGTYNNPSLKSRVTISVDTSKNQFSLKLSYVTAADTAVYYCARQNPRRYASGASDYRGQGTLVTVSS

SEQ ID NO: 19 - protein, 976 aa, Homo sapiens [sequence omitted]

SEQ ID NO: 20 - protein, 8 aa, synthetic construct
GFTFDDYA

SEQ ID NO: 21 - protein, 8 aa, synthetic construct
GFTFANYA

SEQ ID NO: 22 - protein, 8 aa, synthetic construct
GFTFNNYA

SEQ ID NO: 23 - protein, 8 aa, synthetic construct
GFTFADYA

SEQ ID NO: 24 - protein, 8 aa, synthetic construct
GFTFDNYA

SEQ ID NO: 25 - protein, 8 aa, synthetic construct
GFTFHNYA

SEQ ID NO: 26 - protein, 8 aa, synthetic construct
ISWNSGSI

SEQ ID NO: 27 - protein, 16 aa, synthetic construct
AKDSRGYGDYRLGGAY

SEQ ID NO: 28 - protein, 16 aa, synthetic construct
AKDSRGYGDYSRGGAY

SEQ ID NO: 29 - protein, 16 aa, synthetic construct
AKDSRGYGDYSLGGAY

SEQ ID NO: 30 - protein, 16 aa, synthetic construct
AKDSRGYGSYSRGGAY

SEQ ID NO: 31 - protein, 123 aa, synthetic construct
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKDSRGYGDYRLGGAYWGQGTLVTVSS

SEQ ID NO: 32 - protein, 123 aa, synthetic construct
EVQLVESGGGLVKPGGSLRLSCAASGFTFDDYAMHWVRQAPGKGLEWVSGISWNSGSIGYADSVKGRFTISRDNAKNSLYLQMNSLRAEDTALYYCAKDSRGYGDYRLGGAYWGQGTLVTVSS

SEQ ID NO: 33 - protein, 123 aa, synthetic construct
EVQLVESGGGLVQPGRSLRLSCAASGFTFDDYAMHWVRQAPGKGLEWVSGISWNSGSIGYADSVKGRFTISRDNAKKSLYLQMNSLRAEDTALYYCAKDSRGYGDYSRGGAYWGQGTLVTVSS

SEQ ID NO: 34 - protein, 123 aa, synthetic construct
EVQLVESGGGLVQPGRSLRLSCAASGFTFDDYAMHWVRQAPGKGLEWVSGISWNSGSIGYADSVKGRFTISRDNAKNSLYLQMNSLRPEDTALYYCAKDSRGYGDYSLGGAYWGQGTLVTVSS

SEQ ID NO: 35 - protein, 123 aa, synthetic construct
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKDSRGYGDYSLGGAYWGQGTLVTVSS

SEQ ID NO: 36 - protein, 123 aa, synthetic construct
EVQLVESGGGLVQPGRSLRLSCAASGFTFANYAMHWVRQAPGKGLEWVSGISWNSGSIDYADSVKGRFTISRDNAKNSLYLQMNSLRAEDTALYYCAKDSRGYGDYSRGGAYWGQGTLVTVSS

SEQ ID NO: 37 - protein, 123 aa, synthetic construct
EVQLVESGGGLVQPGRSLRLSCAASGFTFNNYAMHWVRQAPGKGLEWVSGISWNSGSIGYADSVKGRFTISRDNAKNSLYLQMNSLRGEDTALYYCAKDSRGYGDYSRGGAYWGQGTLVTVSS

SEQ ID NO: 38 - protein, 123 aa, synthetic construct
EVQLVESGGGLVQPGGSLRLSCAASGFTFADYAMHWVRQAPGKGLEWVSGISWNSGSIGYADSVKGRFTISRDNAKNSLYLQMNSLRAEDTALYYCAKDSRGYGDYSRGGAYWGQGTLVTVSS

SEQ ID NO: 39 - protein, 123 aa, synthetic construct
EVQLVESGGGLVQPGRSLRLSCAASGFTFDNYAMHWVRQAPGKGLEWVSGISWNSGSIGYADSVKGRFTISRDNAKKSLYLQMNSLRAEDTALYYCAKDSRGYGDYSRGGAYWGQGTLVTVSS

SEQ ID NO: 40 - protein, 123 aa, synthetic construct
EVQLVESGGGLVQPGRSLRLSCAASGFTFDNYAMHWVRQAPGKGLEWVSGISWNSGSIGYADSVKGRFTISRDNAKNSLYLQMNSLRAEDTALYYCAKDSRGYGDYSRGGAYWGQGTLVTVSS

SEQ ID NO: 41 - protein, 123 aa, synthetic construct
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKDSRGYGDYSRGGAYWGQGTLVTVSS

SEQ ID NO: 42 - protein, 123 aa, synthetic construct
EVQLVESGGGLVQPGRSLRLSCAASGFTFDDYAMHWVRQAPGKGLEWVSGISWNSGSIGYADSVKGRFTISRDNAKNSLYLQMNSLRPEDTALYYCAKDSRGYGSYSRGGAYWGQGTLVTVSS

SEQ ID NO: 43 - protein, 123 aa, synthetic construct
EVQLVESGGGLVQPGRSLRLSCAASGFTFHNYAMHWVRQAPGKGLEWVSGISWNSGSIGYADSVKGRFTISRDNAKNSLYLQMNSLRAEDTALYYCAKDSRGYGDYSLGGAYWGQGTLVTVSS

SEQ ID NO: 44 - protein, 123 aa, synthetic construct
EVQLVESGGGLVQPGGSLRLSCAASGFTFDDYAMHWVRQAPGKGLEWVSGISWNSGSIGYADSVKGRFTISRDNAKNSLYLQMNSLRAEDTALYYCAKDSRGYGDYSRGGAYCGQGTLVTVSS

SEQ ID NO: 45 - protein, 123 aa, synthetic construct
EVQLVESGGGLVQPGRSLRLSCAASGFTFDNYAMHWVRQAPGKGLEWVSGISWNSGSIGYADSVKGRFTISRDNAKKSLYLQMNSLRAEDTALYYCAKDSRGYGDYSLGGAYWGQGTLVTVSS

SEQ ID NO: 46 - protein, 123 aa, synthetic construct
EVQLVESGGGLVQPGRSLRLSCAASGFTFDDYAMHWVRQAPGKGLEWVSGISWNSGSIGYADSVKGRFTISRDNAKKSLYLQMNSLRAEDTALYYCAKDSRGYGDYRLGGAYWGQGTLVTVSS

SEQ ID NO: 47 - protein, 123 aa, synthetic construct
EVQLVESGGGLVQPGRSLRLSCEASGFTFDDYAMHWVRQAPGKGLEWVSGISWNSGSIGYADSVKGRFTISRDNAKNSLYLQMNSLRAEDTALYYCAKDSRGYGDYRLGGAYWGQGTLVTVSS

SEQ ID NO: 48 - protein, 123 aa, synthetic construct
EVQLVESGGGLVQPGRSLRLSCAASGFTFDDYAMHWVRQAPGKGLEWVSGISWNSGSIGYADSVKGRFTISRDNAKNSLYLQMNSLRPEDTALYYCAKDSRGYGDYRLGGAYWGQGTLVTVSS

SEQ ID NO: 49 - protein, 6 aa, synthetic construct
QSVSSN

SEQ ID NO: 50 - skipped

SEQ ID NO: 51 - protein, 9 aa, synthetic construct
QQYNNWPWT

SEQ ID NO: 52 - protein, 107 aa, synthetic construct
EIVMTQSPATLSVSPGERATLSCRASQSVSSNLAWYQQKPGQAPRLLIYGASTRATGIPARFSGSGSGTEFTLTISSLQSEDFAVYYCQQYNNWPWTFGQGTKVEIK

SEQ ID NO: 53 - protein, 8 aa, synthetic construct
  VARIANT 2: note = Xaa is F or L
  VARIANT 4: note = Xaa is F or I
  VARIANT 5: note = Xaa is D or S
  VARIANT 6: note = Xaa is S or T
GXTXXXYA

SEQ ID NO: 54 - protein, 16 aa, synthetic construct
  VARIANT 6: note = Xaa is S or P
  VARIANT 9: note = Xaa is Y or H
ATGYDXSGXYYGGFDY

SEQ ID NO: 55 - protein, 8 aa, synthetic construct
  VARIANT 5: note = Xaa is D, A, or H
  VARIANT 6: note = Xaa is D or N
GFTFXXYA

SEQ ID NO: 56 - protein, 16 aa, synthetic construct
  VARIANT 9: note = Xaa is D or S
  VARIANT 11: note = Xaa is R or S
  VARIANT 12: note = Xaa is L or R
AKDSRGYGXYXXGGAY

SEQ ID NO: 57 - protein, 330 aa, Homo sapiens [sequence omitted]

SEQ ID NO: 58 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 59 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 60 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 61 - protein, 13 aa, Homo sapiens
ESKYGPPCPSCPA

SEQ ID NO: 62 - protein, 13 aa, synthetic construct
ESKYGPPCPPCPA

SEQ ID NO: 63 - protein, 110 aa, Homo sapiens
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAK

SEQ ID NO: 64 - protein, 110 aa, synthetic construct
APEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAK

SEQ ID NO: 65 - protein, 107 aa, Homo sapiens
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 66 - protein, 107 aa, synthetic construct
GQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 67 - protein, 107 aa, synthetic construct
GQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 68 - protein, 105 aa, Homo sapiens
QDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMD

SEQ ID NO: 69 - protein, 207 aa, Homo sapiens
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 70 - protein, 171 aa, Homo sapiens
MEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNK

SEQ ID NO: 71 - protein, 8 aa, synthetic construct
  VARIANT 3: note = Xaa is V or T
  VARIANT 4: note = Xaa is R or G
  VARIANT 6: note = Xaa is G or S
  VARIANT 7: note = Xaa is S or R
ISXXGXXT

SEQ ID NO: 72 - protein, 45 aa, synthetic construct
TTTPAPRPPTPAPTIASQPLSLRPEACRPAAGGAVHTRGLDFACD

SEQ ID NO: 73 - DNA, 135 bp, synthetic construct
  source 1..135: mol_type = other DNA
actaccacaccagcacctagaccaccaactccagctccaaccatcgcgagtcagcccctgagtctgagacctgaggcctgcaggccagctgcaggaggagctgtgcacaccaggggcctggacttcgcctgcgac

SEQ ID NO: 74 - DNA, 135 bp, synthetic construct
  source 1..135: mol_type = other DNA
accacaacccctgcccccagacctcccacacccgcccctaccatcgcgagtcagcccctgagtctgagacctgaggcctgcaggccagctgcaggaggagctgtgcacaccaggggcctggacttcgcctgcgac